(19) United States
(12) Patent Application Publication
(10) Pub. No.: US 2024/0391961 A1
(43) Pub. Date: Nov. 28, 2024

(54) AUTONOMOUS ORGANISMS FOR SYNTHESIS OF PERMANENTLY PHOSPHORYLATED PROTEINS

(71) Applicant: Oregon State University, Corvallis, OR (US)

(72) Inventors: Richard B. Cooley, Corvallis, OR (US); Ryan A. Mehl, Corvallis, OR (US); Phillip Zhu, Corvallis, OR (US)

(73) Assignee: Oregon State University, Corvallis, OR (US)

(21) Appl. No.: 18/686,249

(22) PCT Filed: Aug. 25, 2022
(86) PCT No.: PCT/US2022/075469

§ 371 Date: Feb. 23, 2024

Related U.S. Application Data
(60) Provisional application No. 63/236,961, filed on Aug. 25, 2021.

(51) Int. Cl. C07K 14/00; C12N 9/10; C12P 21/00

(52) U.S. Cl. CPC C07K 14/00; C12N 9/1096; C12P 21/00; C12Y 206/01019; C07K 2319/90

(57) ABSTRACT

Embodiments of the present disclosure provide compositions and methods for biosynthesizing a stable, functional mimic of phosphoserine. The compositions and methods described herein genetically program a host cell to express a biosynthetic pathway that can synthesize the amino acid, 2-amino-4-phosphobobutyric acid, that is a non-hydrolyzable phosphoserine (nhpSer) because it contains a carbon-phosphorus, e.g., phosphonate, bond. In one embodiment the genetically programed cells express the enzymes of a pathway from a Streptomyces bacterium. In some embodiments, nhpSer is translationally incorporated into protein of interest at one or more programmed UAG amber codons using Genetic Code Expansion (GCE) technology.

### CROSS-REFERENCE(S) TO RELATED APPLICATION(S)

This application claims the benefit of U.S. Provisional Application No. 63/236,961, filed on Aug. 25, 2021.

### STATEMENT OF GOVERNMENT LICENSE RIGHTS

This invention was made with Government support under R01 GM131168 awarded by National Institutes of Health. The Government has certain rights in the invention.

### STATEMENT REGARDING SEQUENCE LISTING

The sequence listing XML associated with this application is provided in XML format and is hereby incorporated by references into the specification. The name of the XML file containing the sequence listing is 3014-P22WO_Seq_List_20220825.xml. The text file is 18 KB; was created on Aug. 25, 2022; and is being submitted via Patent Center with the filing of the specification.

### BACKGROUND

Unless otherwise indicated herein, the materials described in this section are not prior art to the claims in this application and are not admitted as prior art by inclusion in this section.

The majority of the human proteome is phosphorylated at multiple sites. Serine, threonine, and tyrosine residues are primary targets of this post-translational modification (PTM), with nearly 80% of detected phospho-sites being serine. The dynamic and transient nature of protein phosphorylation allows protein signaling pathways to be carefully orchestrated, and imbalances within these systems are key signatures of disease. The inherent reversibility of phosphorylation, however, poses challenges in any effort to study the function of specific phospho-proteins, their interactions with other proteins, and how they modulate signaling systems. It also hinders the ability to develop effective anti-phospho-protein antibodies and limits the capacity to generate new anti-viral and anti-cancer vaccines that target characteristic phospho-protein/peptide signatures since phospho-antigens need to be exposed to living systems rife with phosphatases. Much about phospho-signaling systems, from the atomic to the cellular level, remains mysterious and inaccessible to modern technologies, while opportunities to establish new anti-cancer and anti-viral therapies remain untapped.

Current methodologies use Genetic Code Expansion (GCE) for the site-specific incorporation of hydrolyzable phosphoserine into mammalian cells. Although important technology, the hydrolyzable phosphoserine is susceptible to complete dephosphorylation, which does not address the need for the production and expression of a permanently phosphorylated protein. Other methodologies capable of producing and expressing proteins with non-hydrolyzable phosphoserine (nhpSer) require supplementing the culture media with high concentrations of exogenous nhpSer-leading to high costs. Additionally, the need to supplement the culture media with exogenous nhpSer does not allow for the selected expression in certain cell types or specific cell populations or ensure high bioavailability for efficient production of permanently phosphorylated proteins.

Further, these methodologies do not address the need for a robust, scalable and efficient production of permanently phosphorylated proteins. The compositions and methods disclosed herein address these needs by describing prototype cells that can biosynthesize a stable, functional mimic of phosphoserine and site-specifically encode it into proteins at genetically programmed sites using Genetic Code Expansion.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the forgoing, in one aspect of the invention, the disclosure provides for a method for producing or expressing a protein of interest comprising a non-hydrolyzable phosphoserine (nhpSer). The method can comprise culturing a genetically modified host cell comprising at least one expression vector that can express the protein of interest comprising the nhpSer, wherein the genetically modified host cell comprises a recombinant biosynthetic pathway. The recombinant biosynthetic pathway can comprise at least one heterologous nucleic acid encoding a phosphoenolpyruvate mutase (EC 5.4.2.9); at least one heterologous nucleic acid encoding a 2-phosphonomethylmalate synthase (EC 2.3.3.19); at least one heterologous nucleic acid encoding an aconitate hydratase (EC 4.2.1.3); and at least one heterologous nucleic acid encoding an isocitrate dehydrogenase NAD+(EC 1.1.1.41) and isocitrate dehydrogenase NADP+ (EC 1.1.1.42). The genetically modified host cell can be mutated to produce a non-functional releasing factor 1 (RF1) protein responsible for terminating translation at a UAG amber codon. The genetically modified host cell can further comprise at least one heterologous nucleic acid that encodes an aminoacyl tRNA synthetase (aaRS), wherein the aaRS can charge a tRNA with the nhpSer; at least one heterologous nucleic acid that encodes a tRNA, wherein the tRNA can decode the UAG amber codon; at least one heterologous nucleic acid encoding the protein of interest wherein the amber codon is inserted at a selected position where the non-hydrolyzable phosphoserine is to be inserted. The method can further comprise culturing the genetically modified host cell under conditions such that the nucleic acids encoding the enzymes of the pathway are translated and the non-hydrolyzable-phosphoserine is inserted into the protein of interest and the nucleic acid encoding the protein of interest is translated thereby incorporating into the protein of interest the nhpSer at the selected position.

In another aspect of the invention, the disclosure provides a genetically modified host cell that can produce a protein of interest comprising a non-hydrolyzable phosphoserine (nhpSer). The genetic modification can comprise a recombinant biosynthetic pathway. The recombinant biosynthetic pathway can comprise at least one heterologous nucleic acid encoding a phosphoenolpyruvate mutase (EC 5.4.2.9); at least one heterologous nucleic acid encoding a 2-phosphonomethylmalate synthase (EC 2.3.3.19); at least one heterologous nucleic acid encoding an aconitate hydratase (EC 4.2.1.3); and at least one heterologous nucleic acid encoding an isocitrate dehydrogenase NAD+(EC 1.1.1.41) and/or isocitrate dehydrogenase NADP+ (EC 1.1.1.42); and a recombinant translational system. The recombinant translational system can comprise at least one heterologous nucleic acid encoding a non-functional releasing factor-1 (RF1); at least one heterologous nucleic acid encoding an aminoacyl tRNA synthetase (aaRS); at least one heterologous nucleic acid encoding a tRNA; and at least one heterologous nucleic acid encoding the protein of interest; wherein the genetically modified host cell produces an increased amount of the nh-pSer incorporated into the protein of interest compared to host cells which are not genetically modified.

In some embodiments, the recombinant biosynthetic pathway can further comprise at least one heterologous nucleic acid encoding an isozyme of the isocitrate dehydrogenase NAD+(EC 1.1.1.41) and an isozyme of the isocitrate dehydrogenase NADP+ (EC 1.1.1.42), wherein the isozyme of the isocitrate dehydrogenase NAD+(EC 1.1.1.41) and the isozyme of the isocitrate dehydrogenase NADP+ (EC 1.1.1.42) have a sequence as set forth in SEQ ID NO: 5.

In some embodiments, the recombinant biosynthetic pathway can further comprise at least one heterologous nucleic acid encoding a transaminase, wherein the addition of the transaminase to the recombinant biosynthetic pathway improves the efficiency of nhpSer biosynthesis. In some embodiments, the transaminase can be selected from the group of serine-pyruvate transaminase (EC 2.6.1.51), comprising a sequence as set forth in SEQ ID NO: 6; 4-aminobutyrate-2-oxoglutarate transaminase (EC 2.6.1.19), comprising a sequence as set forth in SEQ ID NO: 7; and 2-aminoethylphosphonate-pyruvate transaminase (EC 2.6.1.37), comprising a sequence as set forth in SEQ ID NO: 8.

In some embodiments, the heterologous nucleic acids can comprise the recombinant biosynthetic pathway are derived from a Streptomyces bacterium.

In some embodiments, at least two of phosphoenolpyruvate mutase (EC 5.4.2.9), 2-phosphonomethylmalate synthase (EC 2.3.3.19), aconitate hydratase (EC 4.2.1.3), isocitrate dehydrogenase NAD+(EC 1.1.1.41), isocitrate dehydrogenase NADP+ (EC 1.1.1.42), an isozyme of isocitrate dehydrogenase NAD+(EC 1.1.1.41), an isozyme of isocitrate dehydrogenase NADP+ (EC 1.1.1.42), and a transaminase can be operatively associated to comprise the biosynthetic pathway. In some embodiments, the phosphoenolpyruvate mutase (EC 5.4.2.9), comprising a sequence as set forth in SEQ ID NO: 1, can be operatively associated with the 2-phosphonomethylmalate synthase (EC 2.3.3.19), comprising a sequence as set forth in SEQ ID NO: 2, to convert phosphonopyruvate into 2-phosphonomethylmalate. In some embodiments, the 2-phosphonomethylmalate synthase (EC 2.3.3.19) can be operatively associated with the aconitate hydratase (EC 4.2.1.3), comprising a sequence as set forth in SEQ ID NO: 3, to convert 2-phosphonomethylmalate into 3-phosphonomethylmalate. In still other embodiments, the aconitate hydratase (EC 4.2.1.3) can be operatively associated with the isocitrate dehydrogenase NAD+(EC 1.1.1.41), the isocitrate dehydrogenase NADP+(EC 1.1.1.42), the isozyme of the isocitrate dehydrogenase NAD+(EC 1.1.1.41), and the isozyme of the isocitrate dehydrogenase NADP+ (EC 1.1.1.42) to convert 3-phosphonomethylmalate into 2-oxo-4-phosphonobutyrate, wherein the isocitrate dehydrogenase NAD+(EC 1.1.1.41) and the isocitrate dehydrogenase NADP+ (EC 1.1.1.42) have a sequence as set forth in SEQ ID NO: 4. In some embodiments, the isocitrate dehydrogenase NAD+(EC 1.1.1.41), the isocitrate dehydrogenase NADP+ (EC 1.1.1.42), the isozyme of the isocitrate dehydrogenase NAD+(EC 1.1.1.41), and the isozyme of the isocitrate dehydrogenase NADP+ (EC 1.1.1.42) can be operatively associated with the transaminase to convert 2-oxo-4-phosphonobutyrate into nhpSer. In some embodiments, the biosynthetic pathway can require the isocitrate dehydrogenase NAD+(EC 1.1.1.41) and the isocitrate dehydrogenase NADP+ (EC 1.1.1.42). In other embodiments, the biosynthetic pathway can require the isozyme of the isocitrate dehydrogenase NAD+(EC 1.1.1.41) and the isozyme of the isocitrate dehydrogenase NADP+ (EC 1.1.1.42).

In some embodiments, one or more of the heterologous genes of the recombinant biosynthetic pathway can be inserted into an expression vector.

In some embodiments, the genetically modified host cell can be a prokaryotic cell or a eukaryotic cell.

In some embodiments, the protein of interest is 14-3-3ζ. In some embodiments, the 14-3-3ζ comprises nhpSer at position 58.

In some embodiments, the tRNA is an orthogonal tRNA that recognizes the UAG amber codon. In other embodiments, the aaRS is an orthogonal aaRS that preferentially aminoacylates the orthogonal tRNA with the nh-pSer to produce the protein of interest containing at least one nh-pSer.

In another aspect of the invention, the disclosure provides a method to identify at least one intracellular protein that stably binds to a monomeric 14-3-3 protein. The method can comprise producing a 14-3-3 nhpSer protein according to the method described above, wherein the nhpSer is expressed at a position to monomerize the dimeric 14-3-3 protein; incubating the monomeric 14-3-3 nhpSer protein in a soluble lysate for a period of time to allow the monomeric 14-3-3 nhpSer protein to bind to at least one intracellular protein in the soluble lysate to form a 14-3-3-nhpSer complex; separating the 14-3-3-nhpSer complex from the soluble cell lysate; and characterizing the 14-3-3-nhpSer complex to identify the stably bound intracellular protein.

In some embodiments, the monomeric 14-3-3 nhpSer protein can be a 14-3-3 isoform selected from one of 14-3-3ζ, 14-3-3β, 14-3-3γ, 14-3-3ε, 14-3-3ƒ, 14-3-3θ, or 14-3-3σ.

In some embodiments, the monomeric 14-3-3 isoform can be 14-3-3ζ and expressing nhpSer at amino acid position 58 (Ser58) in the sequence as set forth in SEQ ID NO: 12 dissociates dimeric 14-3-3ζ into two 14-3-3ζ monomers.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates the PermaPhos technology for the controllable synthesis of non-hydrolyzable phosphoserine (nhpSer). A PermaPhos cell includes a biosynthetic pathway for the controllable intracellular synthesis of nhpSer. The cell uses Genetic Code Expansion to incorporate nhpSer into select proteins during translation leading to the synthesis of homogenous, permanently phosphorylated proteins and peptides.

FIGS. 2A and 2B demonstrate that nhpSer is an accurate mimic of phosphoserine (pSer). FIG. 2A depicts native phosphoserine and non-hydrolyzable phosphoserine. FIG. 2B shows that aspartate and glutamate phosphomimetics are not reliable substitutes of phosphoserine, while non-hydrolyzable phosphoserine recapitulates the size, shape, and charge of phosphoserine.

FIG. 3 illustrates that current pSer and nhpSer Genetic Code Expansion (GCE) systems rely on ΔserB and ΔserC strains to control build up or depletion of pSer, respectively.

FIGS. 4A and 4B illustrate that poor availability of non-canonical amino acids results in low cellular concentration of amino-acylated tRNA_{CUA} and poor competition with Release Factor 1 (RF1). FIG. 4A shows that in RF1(+) expression hosts, amino-acylated tRNA_{CUA} must compete with RF1 and low cellular concentrations of the amino-acylated tRNA_{CUA} leads to premature translational termination and buildup of truncated proteins. FIG. 4B shows that in RF1(−) expression hosts, amino-acylated tRNA_{CUA} must compete with endogenous near-cognate amber suppressing tRNAs that can lead to contaminating protein forms with natural amino acids in place of the ncAAs. In contrast to the current GCE technologies, embodiments of the presently disclosed methods avoid poor availability of non-canonical amino acids by coupling a biosynthetic pathway for the production of nhpSer with GCE technologies.

FIG. 5 is the recombinant biosynthetic pathway for the biosynthesis of nhpSer. As illustrated, starting with the substrate, phosphoenolpyruvate, the enzymes: FrbD (EC 5.4.2.9), FrbC (EC 2.3.3.19), FrbA (EC 4.2.1.3), FrbB (EC 1.1.1.41 and EC 1.1.1.42) and/or FrbE (EC 1.1.1.41 and EC 1.1.1.42), and a cellular transaminase, this biosynthetic pathway catalyzes the biosynthesis of nhpSer.

FIGS. 6A through 6C illustrate the process for the development and screening of T7 promoter variants that increase transcription efficiency of the FrbABCDE proteins. FIG. 6A shows that a library of transcription promoters was generated and later screened. FIG. 6B depicts the screening of the T7 promoter mutants in front of an sfGFP reporter protein, which resulted in four variants that provided expression levels-compared to WT—of 38%, 10%, 4%, and 1%. FIG. 6C shows the nucleotide sequences of selected T7 promoter variants.

FIG. 7 illustrates the diversification of biosynthetic pathways by combinatorial design. As illustrated, different regulatory elements of protein expression and activity can be combined and assembled into expression vectors. (Figure adopted from Jeschek et. al., Combinatorial pathway optimization for streamlined metabolic engineering).

FIGS. 8A through 8D illustrates the standard workflow for screening Frb biosynthetic assemblies. Step 1, generated Frb libraries (FIG. 8A) were co-transformed into BL21(DE3) ΔserC cells (FIG. 8B) along with nhpSer GCE machinery (FIG. 8A) and a sfGFP reporter plasmid containing a TAG codon at position 150 (FIG. 8A). Step 2, clones with a functional Frb assembly should synthesize nhpSer, which is incorporated into sfGFP by the nhpSer GCE machinery causing the cells to fluoresce relative to the production of nhpSer (FIG. 8B) and the yield of sGFP is determined in mg/liter of culture (FIG. 8C). Step 3, isolated fluorescent clones are evaluated for expression in liquid culture. Step 4, additional characterization to confirm nhpSer incorporation. The selection process of Steps 1-4 identifies additional Frb biosynthetic pathway assemblies. Step 5, the new Frb biosynthetic pathway assemblies are used as templates to create next generation libraries, which are then sent through the selection process of Steps 1-4 to identify more efficient pathway assemblies. FIG. 8D also illustrates that clones 1.8, 2.6, and 4C9 are functional biosynthetic assemblies of FrABCDE that enable sf-GFP synthesis and nhpSer incorporation at approximately 3-fold above background. Assemblies 1.8, 2.6, and 4C9 have been identified, other functional assemblies can exist and can be isolated.

FIGS. 9A through 9C illustrate the pSer GCE platform, pSer 3.1G, which is a benchmark for the desired qualities of a prokaryotic PermaPhos organism (recombinantly modified host cell). FIG. 9A shows the N-terminal affinity tag constructs. FIG. 9B shows that product did not co-purify with truncated protein unless expressed in an RF1-deficient expression host. FIG. 9C shows that the purified proteins are homogenously phosphorylated with pSer.

FIG. 10 demonstrates that Bcl-xL expressing nhpSer at position 62 is resistant to hydrolysis by λ-phosphatase (PPase). The upward shift in electrophoretic mobility is dependent on site 62 being phosphorylated. For comparison, pSer62 was fully hydrolyzed.

FIGS. 11A and 11B illustrate custom PermaPhos peptides and an illustrative test application. FIG. 11A shows that PermaPhos peptides can be synthesized in vivo and cleaved to remove solubility fusion tags. FIG. 11B shows how the peptides can be used to study complexes with 14-3-3, a protein known to bind specifically to pSer containing proteins, e.g., 14-3-3 does not bind to non-phosphorylated proteins, and heat shock protein B6 (HSPB6) containing either pSer or nhpSer, and calcineurin phosphatase.

FIGS. 12A through 12C demonstrate that PermaPhos nhpSer is a functional mimic of pSer sufficient to induce full-length heat shock protein B6 (HSPB6) binding to 14-3-3. FIG. 12A shows that the expression constructs were designed to ensure 14-3-3 was pulled down only if it was complexed to HSPB6. FIG. 12B demonstrates that HSPB6 pulled down 14-3-3 when HSPB6 was expressed with pSer and nhpSer at site 16 but not Ser. FIG. 12C depicts Phos-tag gels that confirm the presence of pSer and nhpSer and the nhpSer from PermaPhos pathway 2.6 (clone 2.6) is resistant to phosphatase hydrolysis.

FIGS. 13A and 13B illustrate that PermaPhos cells can be constitutive or light-activated. FIG. 13A shows that for light-activated cells a Frb protein, e.g., FrbD (EC 5.4.2.9) will be placed under control of the light responsive promoter EL222. Activation will lead to nhpSer production and sfGFP expression. FIG. 13B shows the MEK1 pathway which is used herein as a test case for constitutive or photo-controlled activation of a signaling system in PermaPhos Cells.

FIGS. 14A through 14C demonstrate that PermaPhos cells can incorporate nhpSer into a model protein (sfGFP) at one (1×) and two (2×) sites. FIG. 14A shows the yield of protein produced when nhpSer is synthesized by the biosynthetic pathway 4C9 in PermaPhos cells compared to supplementing chemically synthesized nhpSer to the media. FIG. 14B shows the SDS-PAGE and Phos-tag gels of the purified proteins from these cultures. In SDS-PAGE, proteins migrate according to the overall size (molecular weight) and in Phos-tag gels, the phosphate group causes proteins to migrate slower. Specifically, as shown for SDS-PAGE, proteins with pSer or nhpSer-made either by the biosynthetic pathway 4C9 in PermaPhos cells or by supplementing the media with nhpSer—are the same size. In the Phos-tag gel, proteins with nhpSer (biosynthetic or supplemented) migrate slower than the same protein with pSer. Similarly, the Phos-tag gel shows that sfGFP with two nhpSer groups incorporated using PermaPhos cells migrate slower than sfGFP with two native pSer groups. FIG. 14C shows that whole protein mass spectrometry confirms faithful incorporation of nhpSer using PermaPhos technology. For comparison, the equivalent proteins with pSer are included to confirm the approximately 2 Da difference in mass between nhpSer and pSer resulting from the oxygen to methylene substitution. See e.g., FIG. 2.

FIGS. 15A through 15C demonstrates that PermaPhos cells are capable of synthesizing nhpSer-containing peptides. FIG. 15A shows a peptide containing nhpSer was synthesized to act as a specific inhibitor of the phosphatase calcineurin. FIG. 15B demonstrates that the peptide is resistant to dephosphorylation, and as shown in FIG. 15C, approximately 2 mg of the purified peptide can be obtained per liter of culture.

FIGS. 16A through 16D demonstrate that nhpSer is a functional mimic for native pSer as determined by its ability to form stable phosphoserine-dependent complexes. To test this concept, two proteins were expressed simultaneously: (1) 14-3-3, a protein known to bind specifically to pSer containing proteins, e.g., 14-3-3 does not bind to non-phosphorylated proteins, and heat shock protein B6 (HSPB6) containing either pSer or nhpSer. See FIG. 16A. For this test, only HSPB6 contained a purification tag as illustrated in FIG. 16B so that only when HSPB6 forms a stable phosphorylation-dependent complex with 14-3-3 will 14-3-3 co-purify. As illustrated in FIG. 16C, when serine or the traditional phosphomimetic aspartate is incorporated in HSPB6 (WT and Asp), 14-3-3 does not co-purify. When pSer and PermaPhos nhpSer is incorporated, 14-3-3-does co-purify. These results indicate that 14-3-3 requires a phosphorylated protein in order to bind HSPB6 to form the 14-3-3/HSPB6 complex and nhpSer is a functional substitute for pSer. FIG. 16C shows nhpSer was accurately incorporated into HSPB6 based on both SDS-PAGE and Phos-tag gels. Finally, FIG. 16D illustrates size-exclusion chromatography coupled to multi-angle light scattering (SEC-MALS) data confirming that 14-3-3/pSer-HSPB6 and PermaPhos generated 14-3-3/nhpSer HSPB6 complexes are identical in molecular weight and stoichiometry.

FIGS. 17A through 17E demonstrate that nhpSer is a functional mimic for native pSer as determined by its ability to promote enzyme catalytic activity. To test this concept, the glycogen synthase kinase-3 beta (GSK3) enzyme was used because its activity is promoted by binding to substrates that contain pSer. FIGS. 17A and 17B show the Covid-19 nucleocapsid protein (N-protein) with pSer at sites 188 and 206 serve as the substrate for GSK3. If nhpSer is a functional mimic of pSer, it should be able to prime GSK3 activity for further phosphorylations of Ser/Thr. FIG. 17C shows a simplified N-protein construct containing only the linker region (residues 175-210) fused to sfGFP that was used to test this concept. As illustrated in FIG. 17D, GSK3 cannot phosphorylate WT (non-phosphorylated) N-protein. GSK3 also cannot phosphorylate N-protein when S188 and S206 are replaced with the traditional phosphomimetic aspartate. Additionally, as expected, including pSer at positions 188 and 206 of the nucleocapsid protein is sufficient to promote GSK3 phosphorylation. See Phos-tag gel columns S188 pSer and S206 pSer in FIG. 17D, and the mass spectrometry analyses in FIG. 17E. Similarly, PermaPhos generated nhpSer at positions 188 and 206 of the nucleocapsid protein is sufficient to promote GSK3 phosphorylation. See Phos-tag gel columns S188 nhpSer and S206 nhpSer, and the mass spectrometry analyses in FIG. 17E.

FIGS. 18A and 18B demonstrate that 14-3-3 dimerization can be regulated by phosphorylation of a serine residue at the dimer interface, e.g., Ser58. FIG. 18A illustrates a representation of the 14-3-3ζ dimer interface. See FIG. 18A inset for a different orientation of the dimer interface at position Ser58. FIG. 18B illustrates the results from expressing 14-3-3ζ with serine (WT), glutamate, pSer, and nhpSer at residue 58. Size exclusion liquid chromatography coupled to multi-angle light scattering (SEC-MALS) confirmed that WT-14-3-3ζ is a dimer. See FIG. 18B (WT). The S58E phosphomimetic substitution also maintained its dimeric configuration. However, 14-3-3ζ dimerization can be regulated by phosphorylation of serine (Ser58). For example, expressing either pSer or nhpSer at site 58 fully monomerizes 14-3-3ζ. See FIG. 18B (S58pSer) and (S58nhpSer). These data confirm that nhpSer is a functional mimic of pSer because like pSer, nhpSer can monomerize 14-3-3ζ , while the traditional glutamate cannot serve as a functional mimic of pSer.

FIGS. 19A and 19B demonstrate that the pKa2 of nhpSer can be measured in the context of a protein. FIG. 19A illustrates a peptide corresponding to residues 11-19 of the human small heat shock protein B6 (HSPB6) with either pSer or nhpSer genetically fused to a SUMO protein. To determine the pKa2, 31P NMR resonances were measured from pH 4 to 10, and as illustrated in FIG. 19B, plotting the chemical shift as a function of pH produces a sigmoidal curve. The inflection point of the sigmoidal curve corresponds to the pKa2. The pKa2 of pSer is 5.78. See top panel. The pKa2 of nhpSer is 7.00. See bottom panel.

FIG. 20 illustrates that 14-3-3ζ -nhpSer58 is necessary to study the function of pm14-3-3. Phos-tag gel electrophoresis and Western Blot of FLAG-tagged 14-3-3 forms indicate 14-3-3ζ -Ser58 is fully hydrolyzed after incubation in soluble cell-lysate for 120 min, while the 14-3-3ζ -nhpSer58 form is stable (top). All forms of 14-3-3 migrate identically on SDS-PAGE before and after incubation in lysate, confirming the electrophoretic shifts seen in the Phos-tag gel are reflective of their phosphorylation status (bottom). In Phos-tag gels, phosphorylated forms transiently interact with the gel matrix, impeding their ability to migrate through the gel. Phosphonates like nhpSer interact more strongly with the phos-tag acrylamide matrix that phosphonates, and therefore migrate slower than their pSer counterpart.

FIG. 21 illustrates a volcano plot comparing the interactomes of 14-3-3ζ WT and 14-3-3 ζ nhpSer58. For each client protein (represented as a dot) identified as binding to both 14-3-3ζ WT and 14-3-3 ζ nhpSer58, the x-axis value reports its enrichment relative to the other. For example, a value of 3 indicates that protein is enriched 2³, or 8-fold in the 14-3-3 ζ nhpSer58 sample relative to the 14-3-3ζ WT sample. A value of 0 indicates that protein is enriched 2⁰, i.e. not enriched, meaning the client protein was present in identical abundance in both samples. The y-axis reports on the statistical significance of the enrichment.

FIG. 22 illustrates a volcano plot comparing the interactomes of 14-3-3ζ WT and 14-3-3 ζ pSer58. When using 14-3-3 ζ pSer58 as bait for the interactome pulldown, about 2-fold fewer enriched proteins are identified compared to those identified using 14-3-3 ζ nhpSer58 as bait (FIG. 21), and for those identified the fold enrichment is generally lower, consistent with the instability of 14-3-3 ζ pSer58 in cell lysates. These data demonstrate the enhanced utility of 14-3-3 ζ monomerized with nhpSer58 via PermaPhos compared to 14-3-3 ζ monomerized with pSer58.

### DETAILED DESCRIPTION

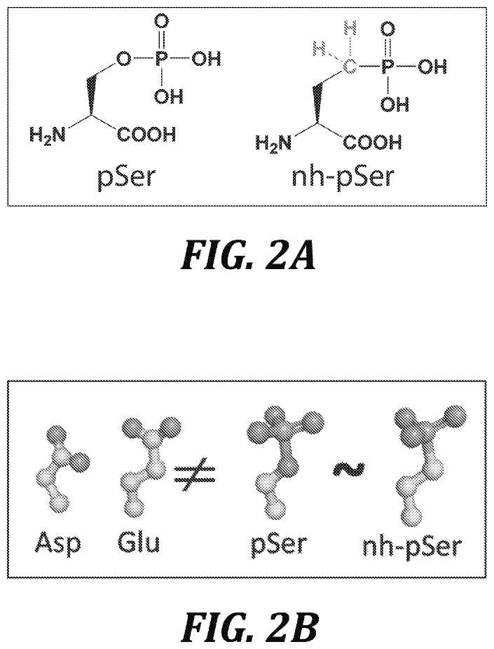

Embodiments of the present disclosure provide compositions and methods for biosynthesizing a stable, functional mimic of phosphoserine, referred to herein as non-hydrolyzable phosphoserine (nhpSer) as illustrated in FIG. 2. The compositions and methods described herein genetically program a cell to express a metabolic pathway comprising at least six enzymes derived from a Streptomyces bacterium. Some embodiments of the composition described herein provide for the biosynthesis of 2-amino-4-phosphobobutyric acid, an amino acid that mimics phosphoserine but contains a carbon-phosphorus, e.g., phosphonate, bond that prevents hydrolysis of the phosphate group. Some embodiments of the methods describe that the 2-amino-4-phosphobobutyric acid is then translationally incorporated into a protein of interest at programmed UAG amber codons using Genetic Code Expansion (GCE) technology.

In some embodiments, the methods describe that charging an amber suppressing tRNA with 2-amino-4-phosphonobutyric acid with a phosphoserine amino-acyl tRNA synthetase, wherein the charged tRNA is then delivered to the ribosome through the elongation factor (EFTu) enzyme where the UAG codon is suppressed and 2-amino-4-phosphonobutyric acid is added to the growing nascent polypeptide chain.

The composition and methods described herein can incorporate at least one 2-amino-4-phosphonobutyric acid residue into a protein. In additional embodiments, the compositions and methods described herein can incorporate 3, 4, 5, and up to an unlimited number of 2-amino-4-phosphobutyric acid residues. The protein can be either an extracellular or intracellular protein. Embodiments of the described methods are applicable in both prokaryotic and eukaryotic cells. The ability to synthesize and incorporate a functional mimic of phosphoserine into a protein of choice overcomes many of the challenges associated with studying the function of phosphoproteins. Embodiments of the composition and methods described herein create compositions not only important in medicine and industry, but also important genetic tools enabling new studies of phosphorylation dependent signaling systems in proteins in vitro and in vivo.

### PermaPhos System

The PermaPhos system enables controllable intracellular synthesis of nhpSer, which is then incorporated into a protein or peptide during translation at a programmed UAG codon, leading to synthesis of homogeneous, permanently phosphorylated proteins and peptides impervious to hydrolysis by phosphatases suitable for in vivo and in vitro applications. As summarized above, aspects of this disclosure include host cells that are engineered to produce a stable, functional, non-hydrolyzable mimic of phosphoserine and site-specifically encode it into a protein or peptide that has been modified at genetically programmed sites using Genetic Code Expansion (GCE).

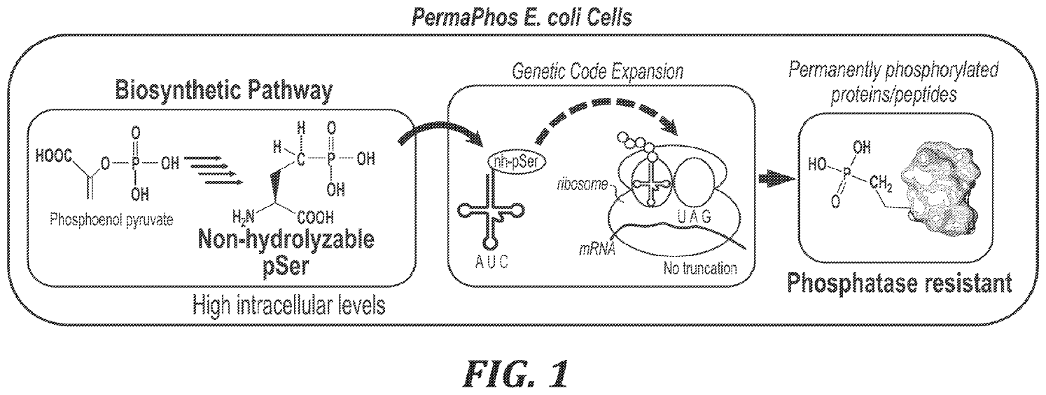

As illustrated in FIG. 1, the PermaPhos system comprises a host cell with (1) a biosynthetic pathway coupled to (2) a Genetic Code Expansion system (recombinant translational system) to produce a protein or peptide (3) comprising at least one nhpSer. Together, the three identified elements comprise the PermaPhos Cell system for synthesizing, incorporating, and expressing a 2-amino-4-phosphonobutyric acid residue, a derivatized amino acid residue that mimics phosphoserine into a protein and peptide of interest. The components of the PermaPhos system will be described further below.

### Host Cell

One aspect of the present disclosure is a genetically modified host cell that can produce a protein of interest comprising at least one nhpSer. The term “genetically modified host cell” as used herein, refers to cells that are engineered to express one or more heterologous nucleic acids, which encode for proteins or peptides that enable the host cell to produce and express nhpSer as described herein. The host cell can be chosen from eukaryotic or prokaryotic cells or cell lines. In some embodiments, the host cell can be a yeast cell, a bacteria cell, an insect cell, a plant cell, or a mammalian cell. In some embodiments, the yeast cell can be Saccharomyces cerevisiae, Pichia pastoris, Hansenula polymorpha, Yarrowia lipolytica, Arxula adeninivorans, Kluyveromyces lactis, or Schizosaccharomyces pombe. In some embodiments, the bacteria cell can be Escherichia coli, Bacillus subtilis, or Salmonella typhimuium, and the like. In some embodiments, the insect cell is, for example, Spodoptera frugiperdai. In some embodiments, the plant cell can be, for example, Arabidopsis T87 cells or Tabacco BY-2 cells. In still other embodiments, the mammalian cell can be a CHO cell, a COS cell, a HEK cell, or a HeLa cell. In some embodiments, suitable cells and cell lines can also include those commonly used in laboratories and/or industrial applications as known by one of ordinary skill in the art.

In some embodiments, the genetic modification can express one or more heterologous nucleic acids encoding for proteins or peptides that enable the host cell to produce and express nhpSer, as described herein. In some embodiments, the heterologous nucleic acids can be integrated stably into the genome of the host cell. In other embodiments, the heterologous nucleic acids can be transiently inserted into the genome of the host cell. As used herein, the term “nucleic acid” includes single-stranded and double-stranded RNA, DNA, and RNA-DNA hybrids.

As used herein, “nucleic acid” also refers to the polymeric form of nucleotides that can include polymeric nucleotides that may vary in length, from about 5 to about 200 nucleotides long, for example. Additionally, a nucleic acid molecule can encode a full-length polypeptide, a polypeptide fragment, a peptide, or the nucleic acid can be non-coding.

As used herein, the term “heterologous” refers to any nucleic acid that is not native to the host cell. A “heterologous nucleic acid” codes for a peptide or protein or its equivalent amino acid sequence, e.g., an enzyme, or its' isoform that is not normally expressed in the host cell and can be expressed in the host cell using an expression system. In some embodiments, the heterologous nucleic acid can also encode for an amino acid sequence that is equivalent to a native amino acid from the host cell. An “equivalent amino acid sequence” is a sequence that is not identical to the native amino acid sequence, but contains modifications, e.g., deletions, substitutions, inversions, insertion, and the like, that do not affect the biological activity of the protein as compared to the native protein. As used herein, the term “native” refers to proteins, peptides, nucleic acids, post-translational modifications, and the like that are intrinsic to the host cell and are not the result of recombinant techniques.

In some embodiments, the genetic modifications can include changes in the host cell to accommodate the biosynthetic pathway. In some embodiments, the changes can include genetically modifying the expression of a specific protein that would reduce the efficiency of the pathway resulting in reduced synthesis of nhpSer. In some embodiments, the changes can include genetically modifying the expression of a specific protein that would increase the efficiency of the pathway resulting in increased synthesis of nhpSer. In some embodiments, the changes can include reducing the expression or function of a specific protein. In other embodiments, the changes can include increasing the expression or potentiating the function of a specific protein. In some embodiments, the genetic modifications target the expression of a heterologous protein. In other embodiments, the genetic modifications target the expression of an autologous protein. Specific embodiments describing genetic modifications to the host cell to accommodate the biosynthetic pathway will be described further below.

In some embodiments, the genetic modification can include changes in the host cell to accommodate a recombinant translational system. In some embodiments, the changes can include genetically modifying the expression of a specific protein that would reduce the translational efficiency of nhpSer. In some embodiments, the changes can include genetically modifying the expression of a specific protein that would increase the translational efficiency of nhpSer into a protein, polypeptide, and/or peptide. In some embodiments, the changes can include reducing the expression or function of a specific protein that can modify the translational efficiency of nhpSer into a protein, polypeptide, and/or peptide. In other embodiments, the changes can include increasing the expression or potentiating the function of a specific protein that can modify the translational efficiency of nhpSer into a protein, polypeptide, and/or peptide. In some embodiments, the genetic modifications can target the expression of a heterologous protein that can modify the translational efficiency of nhpSer into a protein, polypeptide, and/or peptide. In other embodiments, the genetic modifications can target the expression of an autologous protein that can modify the translational efficiency of nhpSer into a protein, polypeptide, and/or peptide. Specific embodiments describing genetic modifications to the host cell to accommodate a translational system will be described further below.

The host cell is cultured under conditions appropriate for the synthesis, production and expression of nhpSer and its incorporation into a predetermined protein, polypeptide, and/or peptide. In some embodiments, the cell culture protocol will depend on the host cell type, e.g., a bacterial or a mammalian cell, of the genetically modified host cell. In some embodiments, the host cell can be cultured under standard or optimized conditions well-known to one of ordinary skill in the art. In some embodiments, the host cell can be cultured under conditions appropriate for the selection of a specific plasmid. In other embodiments, the host cell is cultured under conditions appropriate for recovering nhpSer from the culture media. In some embodiments, standard methods well-known to one of ordinary skill in the art can be used for separation and isolation to recover nhpSer from the cell culture.

The production of a nhpSer is referred to as a yield. As used herein “production” of nhpSer refers to the synthesis of nhpSer and incorporation of nhpSer into a protein of interest during translation followed by isolating and purifying the protein comprising the nhpSer. A method of “producing” an nhpSer refers to the synthesis and incorporation of nhpSer into a protein of interest.

As used herein, the term “yield” refers to the production of nhpSer by a host cell, expressed as mg of nhpSer purified from a liter of culture media. In some embodiments, standard methods well-known to one of ordinary skill in the art for separation and isolation can be adapted as required to increase the efficiency of recovery of nhpSer from the cell culture media. In some embodiments, filtration methods can be used to separate soluble from insoluble fractions of the cell culture media. In other methods, for example, liquid chromatography methods can be used to separate nhpSer from the other soluble components of the cell culture.

As described above, compositions and methods are disclosed that describe the synthesis and incorporation of the nhpSer cell system comprising (1) a biosynthetic pathway, (2) a recombinant translational system, and (3) the product or proteins and peptides comprising nhpSer, all of which are described further below.

### (1) Biosynthetic Pathway (Non-Hydrolyzable Phosphoserine)

One aspect of the present disclosure includes a genetically modified host cell that produces 2-amino-4-phosphonobutyric acid. 2-amino-4-phosphonobutyric acid is an amino acid residue that mimics a phosphoserine residue but contains a carbon-phosphorus, i.e., phosphonate, bond which prevents hydrolysis of the phosphate group. As will be described further below, the 2-amino-4-phosphobutyric amino acid residue is a functional mimic of phosphoserine and will be referred to as non-hydrolyzable phosphoserine (nhpSer).

The biosynthetic pathway is engineered in a host cell from the heterologous expression of at least six proteins which can be derived from, for example, a Streptomyces bacterium. Starting compounds required for the biosynthesis of nhpSer are those compounds native to the host cell. The biosynthetic pathway does not require supplementing growth conditions with exogenous starting compounds. Additionally, the PermaPhos system does not require supplementing the culture media with chemically synthesized nhpSer, as described further below, the biosynthetic pathway can synthesize high intracellular levels of nhpSer.

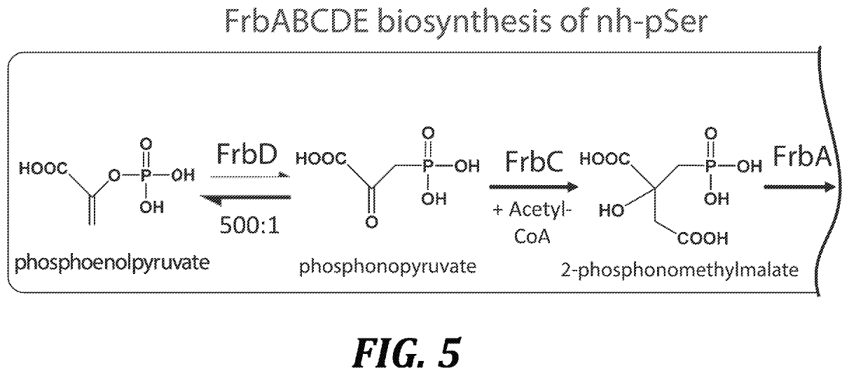

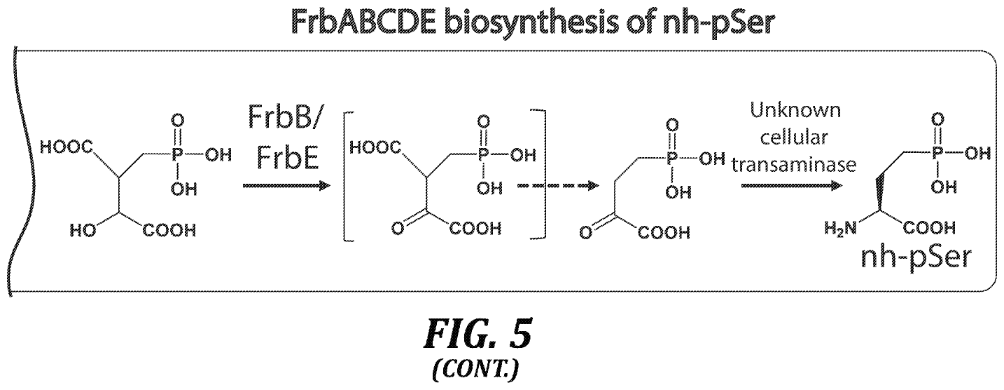

In some embodiments, the genetically modified host cell comprises one or more heterologous enzymes from, for example, the pathway for the production of the fosfomycin derivative FR900098 found in Streptomyces rubellomurinus. In some embodiments, the one or more heterologous enzymes of the FR900098 pathway required for the synthesis of nhpSer include the FrbABCDE biosynthetic pathway enzymes. As used herein, the term “FrbABCDE biosynthetic pathway enzymes” refer to the FrbA enzyme, the FrbB enzyme, the FrbC enzyme, the FrbD enzyme, and the FrbE enzyme. According to the enzyme nomenclature, FrbA is assigned EC number 4.2.1.3 (EC 4.2.1.3). The accepted name of FrbA is aconitate hydratase. FrbA is assigned to the class of lyases, carbon-oxygen lyases, and hydro-lyases. According to the enzyme nomenclature, FrbB is assigned EC number 1.1.1.41 NAD+ dependent (EC 1.1.1.41) and 1.1.1.42 NADP+ dependent (EC 1.1.1.42). The accepted name of FrbB is isocitrate dehydrogenase (NAD+) and isocitrate dehydrogenase (NADP+). FrbB is assigned to the class of oxidoreductases, acting on the CH—OH group of donors; and with NAD+ or NADP+ as an acceptor. According to the enzyme nomenclature, FrbC is assigned EC number 2.3.3.19 (EC 2.3.3.19). The accepted name of FrbC is 2-phosphonomethylmalate synthase. FrbC is assigned to the class of transferases, acyltransferases, and acyl groups converted into alkyl groups on transfer. According to the enzyme nomenclature, FrbD is assigned EC number 5.4.2.9 (EC 5.4.2.9). The accepted name of FrbD is phosphoenolpyruvate mutase. FrbD is assigned to the class of isomerases, intramolecular transferases, and phosphotransferases (phosphomutases). According to the enzyme nomenclature, FrbE is assigned EC number 1.1.1.41 NAD+ dependent (EC 1.1.1.41) and 1.1.1.42 NADP+ dependent (EC 1.1.1.42). The accepted name of FrbE is isocitrate dehydrogenase (NAD+) and isocitrate dehydrogenase (NADP⁺). FrbE is assigned to the class of oxidoreductases, acting on the CH—OH group of donors; and with NAD+ or NADP+ as an acceptor. FrbB isocitrate dehydrogenase NAD+(EC 1.1.1.41) and FrbB isocitrate dehydrogenase NADP+(EC 1.1.1.42) are isozymes of FrbE isocitrate dehydrogenase NAD⁺ (EC 1.1.1.41) and FrbE isocitrate dehydrogenase NADP+(EC 1.1.1.42). As used herein, the term “isozyme” refers to enzymes having a different amino acid sequence but catalyze the same chemical reaction. For example, FrbB isocitrate dehydrogenase NAD⁺ (EC 1.1.1.41) and FrbB isocitrate dehydrogenase NADP+(EC 1.1.1.42) can catalyze the same chemical reaction as FrbE isocitrate dehydrogenase NAD⁺ (EC 1.1.1.41) and FrbE isocitrate dehydrogenase NADP+ (EC 1.1.1.42) (see e.g., FIG. 5), but both enzymes have different amino acid sequences.

In some embodiments, the last step in the nhpSer biosynthetic pathway is catalyzed by the host cell's endogenous transaminase. In some embodiments, the efficiency of the last step in the nhpSer biosynthetic pathway is increased by modifying the expression of the endogenous transaminase. In some embodiments, the genetically modified host cell comprises an autologously expressed transaminase. In some embodiments, the genetically modified host cell comprises a heterologously expressed transaminase. In some embodiments, the heterologous transaminase is serine-pyruvate transaminase. According to the enzyme nomenclature, serine-pyruvate transaminase is assigned EC number 2.6.1.51 (EC 2.6.1.51). Serine-pyruvate transaminase is assigned to the class of transferases, transferring nitrogenous groups, and transaminases. In some embodiments, the heterologous transaminase is 4-aminobutyrate-2-oxoglutarate transaminase. According to the enzyme nomenclature, 4-aminobutyrate-2-oxoglutarate transaminase is assigned EC number 2.6.1.19 (EC 2.6.1.19). 4-aminobutyrate-2-oxoglutarate transaminase is assigned to the class of transferases, transferring nitrogenous groups, and transaminases. In some embodiments, the heterologous transaminase is 2-aminoethylphosphonate-pyruvate transaminase. According to the enzyme nomenclature, 2-aminoethylphosphonate-pyruvate transaminase is assigned EC number 2.6.1.37 (EC 2.6.1.37). 2-aminoethylphosphonate-pyruvate transaminase is assigned to the class of transferases, transferring nitrogenous groups, and transaminases.

In some embodiments, the enzymes of the pathway are introduced into the host cell through the transfection or transformation of the host cell with at least one expression vector. In some embodiments, the expression vector is a plasmid. In some embodiments, the expression vector is a virus.

In some embodiments, at least two of a FrbD (EC 5.4.2.9), FrbC (EC 2.3.3.19), FrbA (EC 4.2.1.3), FrbB (EC 1.1.1.41 and EC 1.1.1.42), FrbE (EC 1.1.1.41 and EC 1.1.1.42) and a transaminase are operatively associated to comprise the biosynthetic pathway. The term “operatively associated” as used herein refers to the cooperative functioning of the enzymes to catalyze the reaction of an initial substrate to the final nhpSer product using a series of reaction steps, wherein each reaction step is catalyzed by a specific enzyme.

In some embodiments, a FrbD enzyme (EC 5.4.2.9) is operatively associated with a FrbC enzyme (EC 2.3.3.19) to convert phosphonopyruvate into 2-phosphonomethylmalate. In some embodiments, a FrbC enzyme (EC 2.3.3.19) is operatively associated with a FrbA enzyme (EC 4.2.1.3) to convert 2-phosphonomethylmalate into 3-phosphonomethylmalate. In some embodiments, a FrbA enzyme (EC 4.2.1.3) is operatively associated with a FrbB enzyme (EC 1.1.1.41 and EC 1.1.1.42) and a FrbE enzyme (EC 1.1.1.41 and EC 1.1.1.42) to convert 3-phosphonomethylmalate into 2-oxo-4-phosphonobutyrate. In some embodiments, a FrbA enzyme (EC 4.2.1.3) is operatively associated with a FrbB enzyme (EC 1.1.1.41 and EC 1.1.1.42) to convert 3-phosphonomethylmalate into 2-oxo-4-phosphonobutyrate. In some embodiments, a FrbA enzyme (EC 4.2.1.3) is operatively associated with a FrbE enzyme (EC 1.1.1.41 and EC 1.1.1.42) to convert 3-phosphonomethylmalate into 2-oxo-4-phosphonobutyrate. In some embodiments, a FrbB enzyme (EC 1.1.1.41 and EC 1.1.1.42) and a FrbE enzyme (EC 1.1.1.41 and EC 1.1.1.42) are operatively associated with a transaminase to convert 2-oxo-4-phosphonobutyrate into nhpSer. In some embodiments, a FrbB enzyme (EC 1.1.1.41 and EC 1.1.1.42) is operatively associated with a transaminase to convert 2-oxo-4-phosphonobutyrate into nhpSer. In still other embodiments, a FrbE enzyme (EC 1.1.1.41 and EC 1.1.1.42) is operatively associated with a transaminase to convert 2-oxo-4-phosphonobutyrate into nhpSer. In some embodiments, the transaminase is an endogenous transaminase. In other embodiments, the transaminase is a heterologous transaminase. In still other embodiments, the transaminase is an autologous transaminase. In some embodiments, the transaminase is serine-pyruvate transaminase (EC 2.6.1.51). In some embodiments, the transaminase is 4-aminobutyrate-2-oxoglutarate transaminase (EC 2.6.1.19). In still other embodiments, the transaminase is 2-aminoethylphosphonate-pyruvate transaminase (EC 2.6.1.37). In certain embodiments, the promoter and/or enhancer elements of the autologous transaminase have been replaced to improve or optimize the production levels of nhpSer.

In some embodiments, SEQ ID NO:1 is an exemplary amino acid sequence for the FrbD enzyme (phosphoenolpyruvate mutase, EC 5.4.2.9). In some embodiments, SEQ ID NO:2 is an exemplary amino acid sequence for the FrbC enzyme (2-phosphonomethylmalate synthase, EC 2.3.3.19). In some embodiments, SEQ ID NO:3 is an exemplary amino acid sequence for the FrbA enzyme (aconitate hydratase, EC 4.2.1.3). In some embodiments, SEQ ID NO:4 is an exemplary amino acid sequence for the FrbB enzyme (isocitrate dehydrogenase NAD⁺, EC 1.1.1.41 and isocitrate dehydrogenase NADP+, EC 1.1.1.42). In some embodiments, SEQ ID NO:5 is an exemplary amino acid sequence for the FrbE enzyme (isocitrate dehydrogenase NAD⁺, EC 1.1.1.41 and isocitrate dehydrogenase NADP+, EC 1.1.1.42). In some embodiments, SEQ ID NO:6 is an exemplary amino acid sequence for the serine pyruvate transaminase (EC 2.6.1.51). In some embodiments, SEQ ID NO:7 is an exemplary amino acid sequence for the 4-aminobutyrate-2-oxoglutarate transaminase (EC 2.6.1.19). In some embodiments, SEQ ID NO:8 is an exemplary amino acid sequence for the 2-aminoethylphosphonate-pyruvate transaminase (EC 2.6.1.37).

### (2) Recombinant Translational System

The recombinant translation system uses the tRNA/aaRS pair to incorporate nhpSer into a growing polypeptide chain, e.g., via a heterologous nucleic acid that encodes a protein of interest, where the nucleic acid comprises an amber codon that is recognized by tRNA. An anticodon loop of the tRNA (CUA) can recognize the amber codon on mRNA and incorporate its nhpSer at the corresponding site in the protein of interest.

One aspect of the present method includes a recombinant translational system comprising at least one heterologous nucleic acid encoding a non-functional releasing factor-1 (RF1); at least one heterologous nucleic acid encoding an aminoacyl tRNA synthetase (aaRS); at least one heterologous nucleic acid encoding tRNA; and at least one heterologous nucleic acid comprising an amber codon encoding a protein of interest, wherein the recombinant translational system incorporates the synthesized nhpSer into the protein of interest at one or more designated locations.

Components of a translation system can include, e.g., ribosomes, tRNAs, synthetases, mRNA, and the like. The term “recombinant translational system” as used herein refers to the components that incorporate an amino acid, e.g., 2-amino-4-phosphonobutyric acid (nhpSer), into a growing polypeptide chain, wherein the components of the translational system are expressed in the host cell specifically for the purpose of incorporating one or more 2-amino-4-phosphonobutyric acid residues into the protein of interest.

In some embodiments, the components of the recombinant translational system are introduced into the host cell through at least one expression vector. In some embodiments, the expression vector is a plasmid. In some embodiments, the expression vector is a virus.

In some embodiments, the recombinant translational system comprises a non-functional RF1. In some embodiment, the host cell is a RF1-deficient strain. As used herein “non-functional,” e.g., make a particular molecule non-operational, means that the target protein is altered in such a way as to decrease or eliminate the activity of the protein in the host cell. In some embodiments, methods to make a particular protein non-functional result in a reduction of protein activity by at least 10%, 20%, 30%, 40%, 50%, 60%, 70%, 80%, 90%, or 100% compared to the activity of a functional protein.

In some embodiments, the recombinant translational system comprises a tRNA. In some embodiments, the tRNA is an orthogonal tRNA. As used herein “orthogonal tRNA” is a tRNA that is orthogonal to a translational system of interest. In some embodiments, the tRNA can exist charged with an amino acid, or in an uncharged state. The tRNA described herein is used to insert any amino acid, whether natural or unnatural, into a growing polypeptide, during translation, in response to a stop codon. In some embodiments, the tRNA can incorporate nhpSer into the protein of interest that is encoded by a nucleic acid that comprises a stop codon that is recognized by the tRNA. As described further below, in some embodiments, the stop codon is an amber codon.

The term “protein of interest” refers to any protein, peptide, polypeptide, or fragment thereof, the modification of which may be deemed desirable for any reason, e.g., has the relevant expression or activity for evaluating the incorporation of nhpSer as determined by one of ordinary skill in the art. These proteins, peptides, polypeptides, or fragments thereof, include extracellular or intracellular proteins with at least one native phosphorylation site, as disclosed and described herein. As used herein, the terms “polypeptide,” “peptide,” “protein,” or “enzyme” are interchangeable and refer to a biomolecule composed of amino acids of any length linked by a peptide bond.

In some embodiments, the recombinant translational system comprises an aminoacyl-tRNA synthetase (aaRS). The aaRS is an enzyme that aminoacylates tRNA with an amino acid residue in a translational system of interest. In some embodiments, the aaRS is an orthogonal aaRS. The orthogonal aaRS is an enzyme that preferentially aminoacylates an orthogonal tRNA with an amino acid residue in a translational system of interest.

The term “orthogonal” refers to a molecule that fails to function when paired with an endogenous cellular component or functions with reduced efficiency compared to the corresponding endogenous cellular component. An orthogonal molecule lacks a functionally normal, naturally occurring endogenous complementary molecule in the cell or translational system. For example, an orthogonal tRNA in a cell is aminoacylated by any endogenous aaRS of the cell with reduced or even undetectable efficiency, when compared to aminoacylation of an endogenous tRNA by the endogenous aaRS. In another example, an orthogonal aaRS aminoacylates any endogenous tRNA in a cell of interest with reduced or even undetectable efficiency, as compared to aminoacylation of the endogenous tRNA by a complementary endogenous aaRS.

In some embodiments, the recombinant translational system comprises a protein of interest encoded by at least one heterologous nucleic acid comprising an amber codon (UAG), wherein the tRNA/aaRS pair act to suppress the amber codon allowing for incorporation of nhpSer.

A “codon” is three nucleotides that encode for a specific amino acid or the termination of translation, e.g., stop codon. In some cases, heterologous gene expression can be improved through the use of codons that correlate with the host cell's tRNA level. These codons are referred to as optimized codons, which improve the speed and accuracy of translation. In some embodiments, the nucleic acid sequence encoding the protein of interest is a codon optimized version of the wild-type gene. An “amber codon” refers to a codon that is recognized by tRNA in the translation process and not the corresponding endogenous tRNA. The tRNA anticodon recognizes the amber codon on mRNA and incorporates its amino acid, e.g., nhpSer, at this site in the polypeptide. In some embodiments, the stop codon is an ochre codon (UAA). In some embodiments, the stop codon is an opal codon (UGA).

The phrase “suppressing the amber codon” refers to the tRNA/aaRS recognizing an amber codon and loading an amino acid residue in response to the amber codon. In the absence of a tRNA/aaRS pair that is not specific for an amber codon, the amber codon is not translated, blocking production of a polypeptide that would have been translated from the nucleic acid.

### (3) Proteins/Peptides

The PermaPhos cell system produces homogenous, permanently phosphorylated proteins and/or peptides that are impervious to hydrolysis by phosphatases, wherein the nhpSer containing proteins and/or peptides of interest are suitable for in vivo and in vitro application. In some embodiments, the protein and/or peptide of interest can be any protein and/or peptide that is capable of being expressed in the PermaPhos cell system. In some embodiments, the protein and/or peptide of interest can be any extracellular or intracellular protein and/or peptide. In some embodiments, the protein and/or peptide of interest can be any 14-3-3 protein. In some embodiments, the protein and/or peptide of interest can be any isoform of 14-3-3. In some embodiments, the isoform is 14-3-3ζ. In some embodiments, the isoform is 14-3-30. In some embodiments, the isoform is 14-3-3γ. In some embodiments, the isoform is 14-3-3ε. In some embodiments, the isoform is 14-3-3η. In some embodiments, the isoform is 14-3-30. In some embodiments, the isoform is 14-3-3σ.

In other embodiments, the protein and/or peptide of interest can contain at least one native phosphorylation site. In some embodiments, the 14-3-3 protein is phosphorylated at Ser58 (i.e., ζ isoform numbering). In other embodiments, the 14-3-3 isoform is phosphorylated at a position equivalent to Ser58 of the C isoform.

In some embodiments, the protein and/or peptide of interest can be a monomer of a dimeric protein. For example, in some embodiments, phosphorylation of Ser58 at the 14-3-3 dimer interface causes the dimeric 14-3-3 to dissociate into individual monomers.

In some embodiments, the protein and/or peptide of interest can be any protein and/or peptide that can bind to any 14-3-3 protein. See e.g., Tables 1 and 2. In other embodiments, the protein and/or peptide of interest can be any protein and/or peptide that can bind to a WW domain. In some embodiments, the protein and/or peptide of interest can be any protein and/or peptide that can bind to a polo box domain. In some embodiments, the protein and/or peptide of interest can be any protein and/or peptide that can bind to a BRCT domain. In still other embodiments, the protein and/or peptide of interest can be any kinase. In some embodiments, the protein and/or peptide of interest can be any transcription factor. In some embodiments, the protein and/or peptide of interest can be any viral protein and/or peptide.

Embodiments of specific components of the biosynthetic pathway were described previously. The following disclosure describes specific embodiments for developing a nhpSer biosynthetic pathway.

### Combinatorial Assembly of the Biosynthetic Pathway

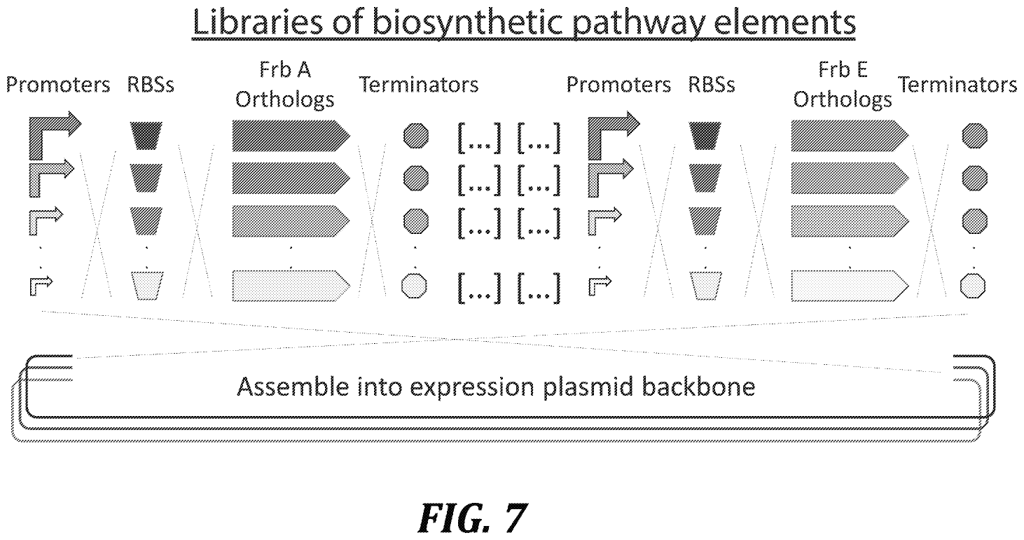

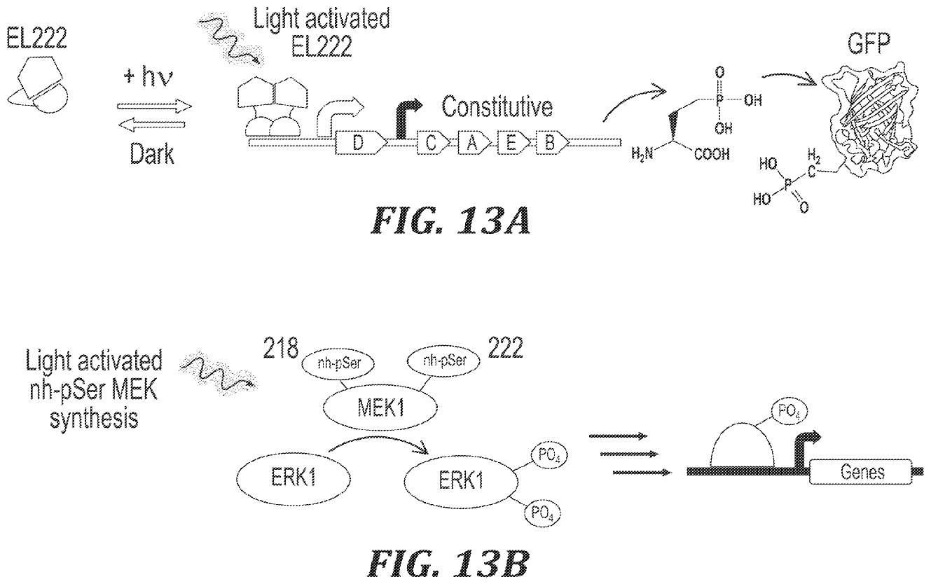

To assembly the biosynthetic pathway, a library with all five, codon-optimized enzymes-FrbABCDE-were each placed under the control of a transcriptional promoters as illustrated in FIG. 7. This strategy can be expanded to include variations of other biosynthetic pathway elements, including different types of promoters, e.g., constitutive or photo-controlled, different strength ribosome binding sites, transcriptional terminators, as well as orthologs or mutants of the FrbABCDE biosynthetic pathway enzymes. In some embodiments, the PermaPhos cells can be light activated as illustrated in FIG. 13(A). In some embodiments, a FrbD enzyme (EC 5.4.2.9) will be placed under control of, for example, the light responsive promoter EL222 and activation will lead to nhpSer production and sfGFP expression as demonstrated in FIG. 13(A). In other embodiments, the PermaPhos cells will be activated by constitutive promoters.

### Screening for Functional Biosynthetic Enzyme Assemblies

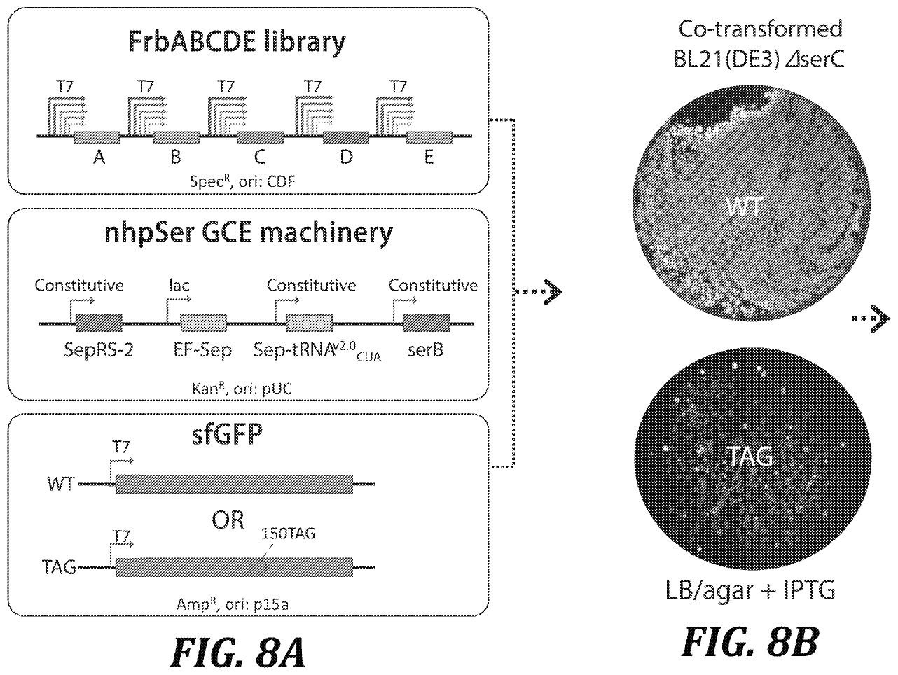

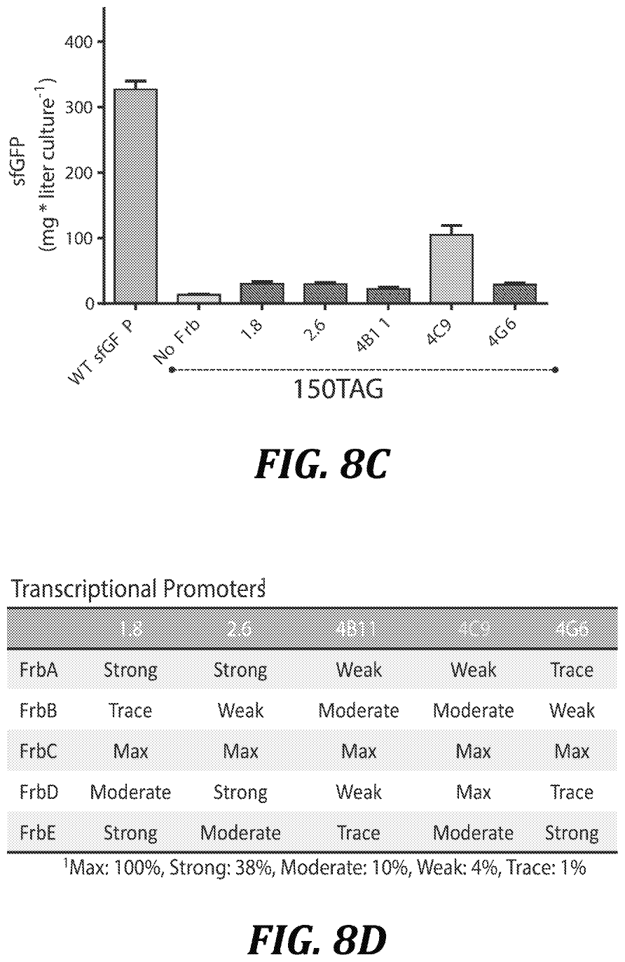

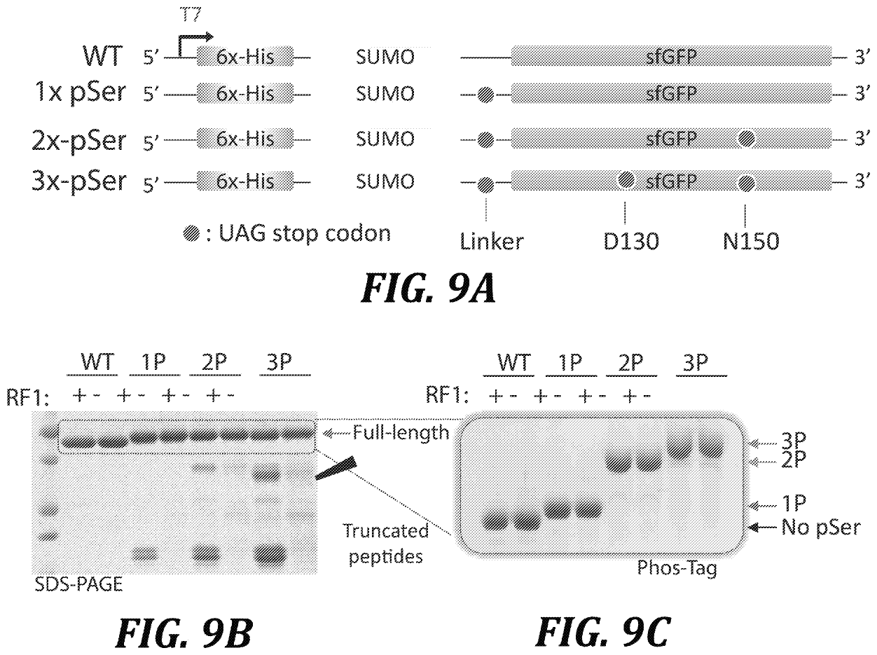

To screen for functional biosynthetic enzyme pathway assemblies, biosynthetic libraries were co-transformed into BL21(DE3) ΔserC cells along with the validated nhpSer GCE machinery and a sfGFP reporter plasmid containing a TAG codon at amino acid position 150 as illustrated in FIG. 8. Clones with a functional biosynthetic enzyme assembly synthesize nhpSer, which is then incorporated into sfGFP by the nhpSer GCE machinery causing the cells to fluoresce relative to the production of nhpSer. Small libraries (<10,000) can be screened by evaluating fluorescence of individual colonies on agar plates. Larger libraries (>10,000) can be screened by fluorescence-activated cell sorting (FACS). Cells expressing empty biosynthesis plasmids with wild-type sfGFP and sfGFP-150nhpSer (made by supplementing the media with an amino acid) served as parallel expression controls. Isolated fluorescent clones are evaluated for expression in liquid culture. A positive hit is considered having at least 2-fold fluorescence above background.

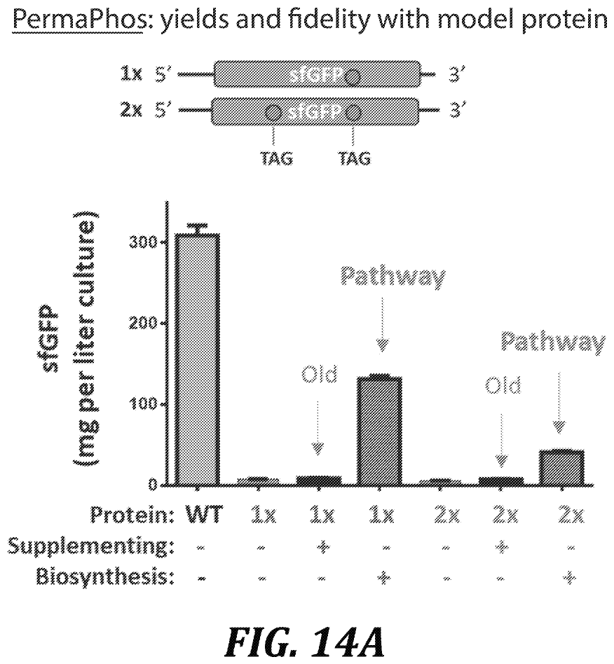

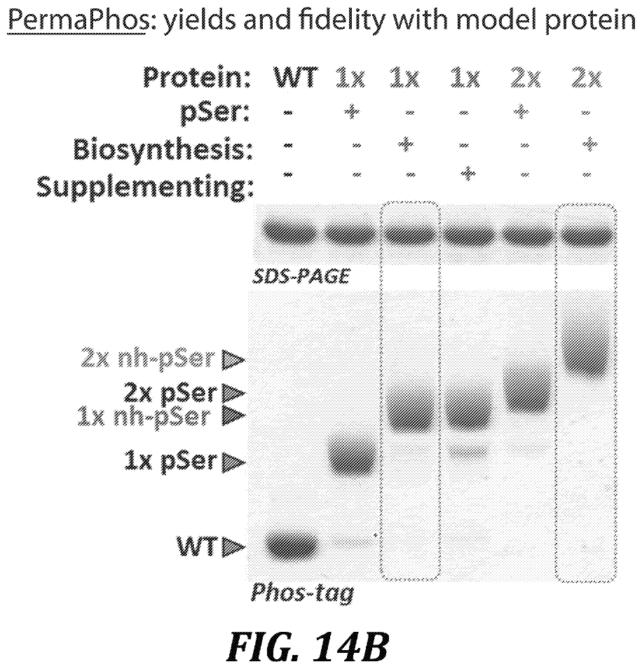

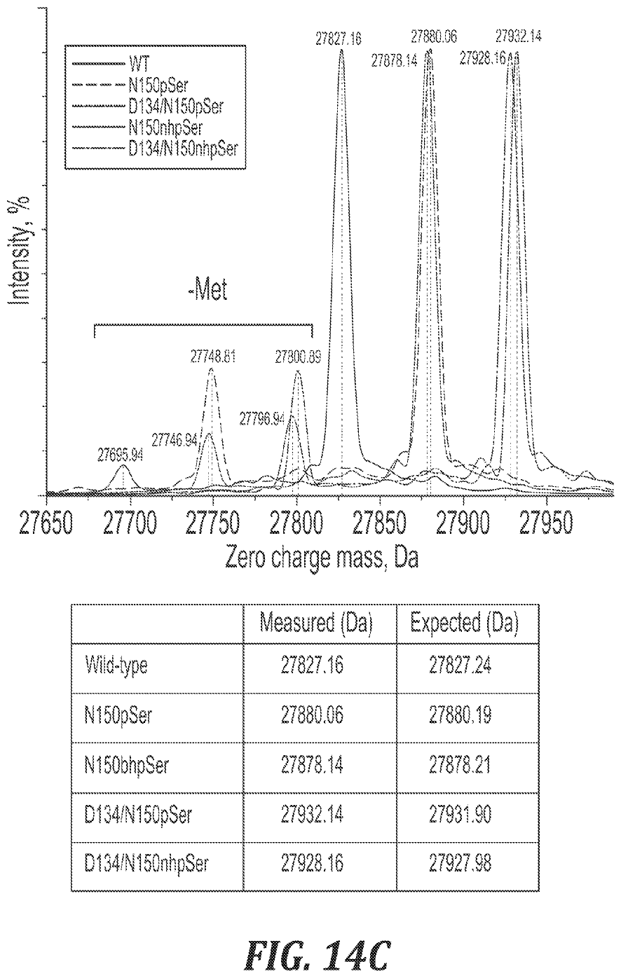

The screens have identified at least three biosynthetic enzyme assemblies that enable sfGFP synthesis at greater than 3-fold above background, which were chosen for further characterization (referred to as clones 1.8, 2.6, and 4C9 as illustrated in FIG. 8). Clones 1.8, 2.6, and 4C9 are examples of embodiments that are functional. Additional optimization can be carried out to identify and isolate more efficient clones. In some embodiments, methods are described for screening Frb biosynthetic assemblies to identify more efficient pathway assemblies. See FIG. 8. Sequencing revealed similar promoters in front of the individual biosynthetic enzyme genes. The FrbC enzyme (EC 2.3.3.19) was expressed at maximal levels in all clones, presumably as a means to more effectively drive forward metabolic flux from the unfavorable equilibrium of the FrbD enzyme (EC 5.4.2.9) reaction. See biosynthetic pathway, FIG. 5. To confirm nhpSer incorporation, whole-protein mass spectrometry and Phos-tag gel electrophoresis were used. With Phos-tag gels, phosphorylated proteins migrate slower and, conveniently, proteins with nhpSer migrate slightly slower than the same protein with native pSer. Incorporation of the nhpSer made via Frb clone 4C9 was confirmed by Phos-tag electrophoresis and whole-protein mass spectrometry as demonstrated in FIG. 14(B—C). As illustrated in FIG. 14(B—C) near homogenous (>95%) incorporation of nhpSer was obtained. Yields of sfGFP-150-nhpSer from these biosynthetic enzyme pathways were approximately 100 mg per liter of culture as shown in FIG. 14(A). Because nhpSer is biosynthesized in this system, it means that E. coli expresses a functional transaminase to complete the last step. The lack of nhpSer expression in the ΔserA strain indicates serC is not the unidentified transaminase. These results demonstrate the feasibility of the PermaPhos technology by showing that the FrbABCDE biosynthetic cluster is active and can be used for nhpSer synthesis.

### Identifying a Minimal Set of Pathway Components

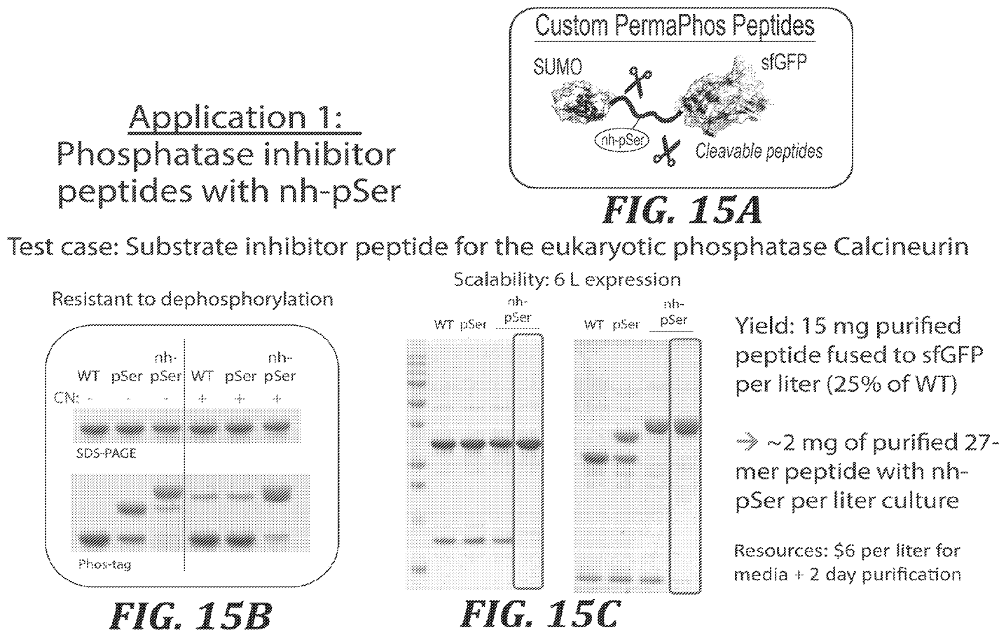

The ideal biosynthetic enzyme assembly expresses all critical and no superfluous components. It is unknown whether FrbB (EC 1.1.1.41 and EC 1.1.1.42) or FrbE (EC 1.1.1.41 and EC 1.1.1.42) is required, or both. See biosynthetic pathway, FIG. 5. Therefore, FrbB (EC 1.1.1.41 and EC 1.1.1.42) and FrbE (EC 1.1.1.41 and EC 1.1.1.42) will be knocked out of the biosynthetic enzyme assemblies 1.8, 2.6, and 4C9, and then sfGFP production and nhpSer incorporation will be evaluated as illustrated in FIG. 15. Clones 1.8, 2.6. and 4C9 are examples of embodiments that are functional. Additional optimization can be carried out to identify more efficient clones and similar experiments will be performed to determine whether the more efficient biosynthetic pathways require both of, or just one of either FrbB (EC 1.1.1.41 and EC 1.1.1.42) or FrbE (EC 1.1.1.41 and EC 1.1.1.42).

Further, overexpression of a functional transaminase could enhance nhpSer biosynthesis. To address this, combinatorial libraries of biosynthetic enzyme assemblies with transaminases will be made. Specifically, 4-aminobutyrate-2-oxoglutarate transaminase from E. coli, known to produce nhpSer from its corresponding 2-oxoacid, will be tested. Alternatively, there are a plethora of phosphonate transaminases found in Streptomyces that can be tested. These assemblies will be synthesized as described in FIG. 7, screened as described in FIG. 8, and their efficiencies and fidelities assessed as described in FIG. 15.

### Evolution of the Enzyme Biosynthetic Pathway

With the minimal set of components identified, extended combinatorial library assemblies targeting the following areas (1-3 described below) will be conducted. From these prokaryotic screens an extensive understanding will be developed regarding what components of the pathway are critical and optimal for efficient nhpSer biosynthesis.

(1) Genetic fusions of FrbC (EC 2.3.3.19) and FrbD (EC 5.4.2.9)

By ensuring FrbC (EC 2.3.3.19) remains in close physical proximity to FrbD (EC 5.4.2.9), metabolic flux through this unfavorable equilibrium can be driven more effectively, thereby ensuring more efficient nhpSer synthesis. Indeed, many phosphoenolpyruvate mutase enzymes exist in nature as a genetic fusion with downstream proteins for this purpose.

### (2) Orthologs of Biosynthetic Enzymes

Searching the genomes of related Streptomyces bacteria using an amino acid or nucleic acid sequence search tool such as, for example, BLAST (blastx or blastn) reveals orthologs of FrbABCDE proteins. With modest differences in sequences, they can have widely different expression properties and activities. Bottlenecks caused by poor or insoluble expression of each biosynthetic enzyme can be identified by Western-blot and addressed with new orthologs. Synthetic DNA fragments encoding potentially better expressing, more active orthologs can be assembled into a library for selection and characterization. A library with three different orthologs for each biosynthetic enzyme with five different promoters can be targeted which provides for 5⁵×3⁵=760,000 unique combinations. These library sizes are tractable in size to assemble and screen either by plating or by FACS.

(3) Additional pathway fragments: Constitutive promoters, RBSs, and terminators.

Promoters regulate gene expression. Promoter regions are typically found in the flanking DNA upstream from the coding sequences in both prokaryotic and eukaryotic cells. A promoter sequence provides for regulation of transcription of the downstream gene sequence and typically includes from about 50 to about 2,000 nucleotide base pairs. Promoter sequences can also contain regulatory elements such as enhancer sequences that can influence the level of gene expression. Some isolated promoter sequences can provide for gene expression of heterologous DNA sequences, that is a DNA different from the native or homologous DNA.

Promoter sequences can be strong or weak, or inducible. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. For example, a bacterial promoter such as the Ptac promoter can be induced to vary levels of gene expression depending on the level of iso thiopropyl galactoside added to the transformed cells.

Promoters can also provide for tissue specific or developmental regulation. In some embodiments, an isolated promoter sequence that is a strong promoter for heterologous DNAs is advantageous because it provides for a sufficient level of gene expression for easy detection and selection of transformed cells and provides for a high level of gene expression when desired.

In some embodiments, constitutive promoters of the biosynthetic enzymes can be advantageous to ensure an adequate intracellular pool of nhpSer at the start of target protein synthesis and can also be used in eukaryotic pathway development. Sequences for the attenuated, prokaryotic and eukaryotic constitutive promoters have been described and are well known in the art.

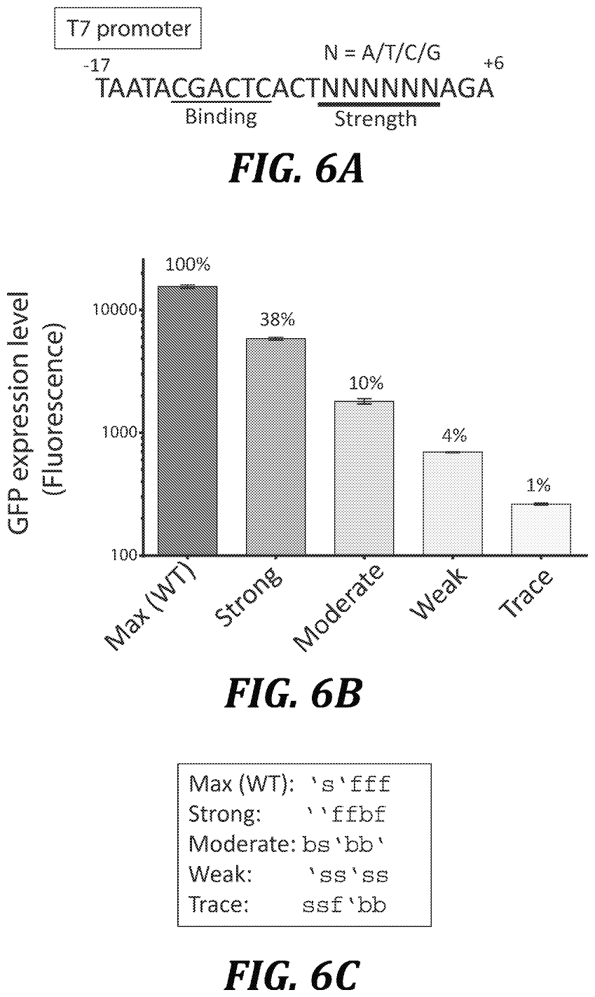

In some embodiments, a set of promoter variants with attenuated transcriptional efficiencies was used to modulate enzyme activity. In some embodiments, the activity of a specific enzyme activity along the biosynthetic pathway can be modulated by, for example, a T7 promoter. To modulate the enzyme activity along the biosynthetic pathway, variants of the T7 promoter conferring increasingly attenuated transcriptional efficiencies spanning two-orders of magnitude were identified. To identify these T7 promoters, a T7 promoter library was created and screened. The T7 promoter mutants were placed in front of a sfGFP reporter protein. These plasmids were transformed into BL21(DE3) ΔserC cells and plated on LB/agar plates with a transcriptional inducer, and colonies with varying fluorescence were picked and evaluated for sfGFP expression in liquid culture as illustrated in FIG. 6, four T7 variants provided GFP expression levels, compared to WT, of 38%, 10%, 4%, and 1%.

Variants of ribosome binding sites and transcriptional terminators can also be screened. In all cases, combinatorial library sizes will be designed to be below 10⁶ to keep assembly and screening manageable.

Methods to Characterize and Evaluate nhpSer Synthesis and Incorporation

In some embodiments, methods are disclosed to confirm synthesis of PermaPhos nhpSer. The method can comprise the steps of: (a) adding a purification tag to nhpSer; (b) culturing a population of PermaPhos cells capable of producing a protein of interest comprising a tagged nhpSer; (c) recovering the protein of interest comprising the tagged nhpSer using purification techniques well-known to one of ordinary skill in the art; and (d) determining yield of PermaPhos nhpSer protein production as mg of tagged nhpSer protein produced per liter of culture media.

In some embodiments, methods are disclosed to confirm incorporation of PermaPhos nhpSer in a protein of interest. The method can comprise the step of recovering PermaPhos nhpSer protein from culture media. In some embodiments, confirmation of successful incorporation of nhpSer can include the step of comparing the molecular weight of a PermaPhos nhpSer protein to a protein with pSer. In some embodiments, the molecular weight between a PermaPhos nhpSer protein and a protein with a pSer can be determined by mass spectrometry according to methods well known to one of ordinary skill in the art. In other embodiments, confirmation of successful incorporation of nhpSer can include the step of comparing the level of phosphorylation. In some embodiments, the level of phosphorylation between a PermaPhos nhpSer protein and a protein with pSer can be compared by running the proteins on a Phos-tag gel according to methods well known to one of ordinary skill in the art. In still other embodiments, confirmation of successful incorporation of nhpSer can include the step of determining molecular weight and stoichiometry. In some embodiments, the molecular weight and stoichiometry between a PermaPhos nhpSer protein and a protein with pSer can be compared by the use of size-exclusion chromatography coupled to multi-angle light scattering (SEC-MALS) according to methods well known to one of ordinary skill in the art.

In some embodiments, methods are disclosed to confirm the stability of PermaPhos nhpSer in a protein of interest. The method can comprise the step of determining resistance to hydrolysis by a phosphatase. In some embodiments, the step can include running the PermaPhos nhpSer in a protein of interest on a Phos-tag gel after exposure to a phosphatase. In some embodiments, the phosphatase can be calcineurin. In some embodiments, the phosphatase can be λ-phosphatase. In other embodiments, the phosphatase can be selected according to the knowledge of one of ordinary skill in the art.

Embodiment 1. A genetically modified host cell that can produce a protein of interest comprising a non-hydrolyzable phosphoserine (nh-pSer), wherein the genetic modification comprises: a recombinant biosynthetic pathway, wherein the recombinant biosynthetic pathway comprises: at least one heterologous nucleic acid encoding a phosphoenolpyruvate mutase (EC 5.4.2.9); at least one heterologous nucleic acid encoding a 2-phosphonomethylmalate synthase (EC 2.3.3.19); at least one heterologous nucleic acid encoding an aconitate hydratase (EC 4.2.1.3); and at least one heterologous nucleic acid encoding an isocitrate dehydrogenase NAD⁺ (EC 1.1.1.41) and/or isocitrate dehydrogenase NADP+ (EC 1.1.1.42); and a recombinant translational system, wherein the recombinant translational system comprises: at least one heterologous nucleic acid encoding a non-functional releasing factor-1 (RF1); at least one heterologous nucleic acid encoding an aminoacyl tRNA synthetase (aaRS); at least one heterologous nucleic acid encoding a tRNA; and at least one heterologous nucleic acid encoding the protein of interest; wherein the genetically modified host cell produces an increased amount of the nh-pSer incorporated into the protein of interest compared to host cells which are not genetically modified.

Embodiment 2. A method for producing or expressing a protein of interest comprising a non-hydrolyzable phosphoserine (nh-pSer), the method comprising: culturing a genetically modified host cell comprising at least one expression vector that can express the protein of interest comprising the nh-pSer, wherein the genetically modified host cell comprises a recombinant biosynthetic pathway comprising: at least one heterologous nucleic acid encoding a phosphoenolpyruvate mutase (EC 5.4.2.9); at least one heterologous nucleic acid encoding a 2-phosphonomethylmalate synthase (EC 2.3.3.19); at least one heterologous nucleic acid encoding an aconitate hydratase (EC 4.2.1.3); and at least one heterologous nucleic acid encoding an isocitrate dehydrogenase NAD⁺ (EC 1.1.1.41) and isocitrate dehydrogenase NADP+ (EC 1.1.1.42); and wherein the genetically modified host cell is mutated to produce a non-functional releasing factor 1 (RF1) protein responsible for terminating translation at a UAG amber codon; wherein the genetically modified host cell comprises: at least one heterologous nucleic acid that encodes an aminoacyl tRNA synthetase (aaRS), wherein the aaRS can charge a tRNA with the nh-pSer; at least one heterologous nucleic acid that encodes a tRNA, wherein the tRNA can decode the UAG amber codon; at least one heterologous nucleic acid encoding the protein of interest wherein the amber codon is inserted at a selected position where the non-hydrolyzable phosphoserine is to be inserted; and culturing the genetically modified host cell under conditions such that the nucleic acids encoding the enzymes of the pathway are translated and the non-hydrolyzable-phosphoserine is inserted into the protein of interest and the nucleic acid encoding the protein of interest is translated thereby incorporating into the protein of interest the nh-pSer at the selected position.

Embodiment 3. A method of making a scalable and autonomous genetically modified host cell capable of producing a protein of interest comprising a non-hydrolyzable phosphoserine (nh-pSer), the method comprising: engineering in the genetically modified host cell a recombinant biosynthetic pathway comprising at least one expression vector that can express the protein of interest comprising the nh-pSer, wherein the expression vector comprises: at least one heterologous nucleic acid encoding a phosphoenolpyruvate mutase (EC 5.4.2.9); at least one heterologous nucleic acid encoding a 2-phosphonomethylmalate synthase (EC 2.3.3.19); at least one heterologous nucleic acid encoding an aconitate hydratase (EC 4.2.1.3); and at least one heterologous nucleic acid encoding an isocitrate dehydrogenase NAD⁺ (EC 1.1.1.41) and isocitrate dehydrogenase NADP+ (EC 1.1.1.42); and engineering in the cell a recombinant translation system comprising: a mutation to produce a non-functional releasing factor 1 (RF1) protein responsible for terminating translation at a UAG amber codon; at least one heterologous nucleic acid encoding an aminoacyl tRNA synthetase (aaRS), wherein the aaRS can charge a tRNA with the nh-pSer; at least one heterologous nucleic acid encoding a tRNA, wherein the tRNA can decode the UAG amber codon; at least one heterologous nucleic acid encoding the protein of interest wherein an UAG amber codon is inserted where the nh-pSer is to be inserted; and wherein the recombinant biosynthetic pathway converts a starting compound to the nh-pSer and the recombinant translational system incorporates into the protein of interest the nh-pSer at the selected position.

Embodiment 4. The genetically modified host cell of embodiment 1 and the method of embodiments 2 and 3, wherein the recombinant biosynthetic pathway further comprises at least one heterologous nucleic acid encoding an isozyme of the isocitrate dehydrogenase NAD⁺ (EC 1.1.1.41) and an isozyme of the isocitrate dehydrogenase NADP+ (EC 1.1.1.42).

Embodiment 5. The genetically modified host cell of embodiment 1 and the method of embodiments 2 and 3, wherein the heterologous nucleic acid encoding the protein of interest is inserted into a vector that has at least one tag designed to fuse to the N-terminus or C-terminus of the protein of interest.

Embodiment 6. The genetically modified host cell of embodiment 1 and the method of embodiments 2 and 3, wherein the expression vector further comprises at least one nucleic acid encoding a transaminase, wherein the addition of the transaminase to the recombinant biosynthetic pathway improves the efficiency of nhpSer biosynthesis.

Embodiment 7. The genetically modified host cell of embodiment 1 and the method of embodiments 2 and 3, wherein the nucleic acid encoding the transaminase is autologous or heterologous.

Embodiment 8. The genetically modified host cell of embodiment 1 and the method of embodiments 2 and 3, wherein the heterologous transaminase is selected from the group of serine-pyruvate transaminase (EC 2.6.1.51), 4-aminobutyrate-2-oxoglutarate transaminase (EC 2.6.1.19), and 2-aminoethylphosphonate-pyruvate transaminase (EC 2.6.1.37).

Embodiment 9. The genetically modified host cell of embodiment 1 and the method of embodiments 2 and 3, wherein the serine-pyruvate transaminase (EC 2.6.1.51) has a SEQ ID NO. 6.

Embodiment 10. The genetically modified host cell of embodiment 1 and the method of embodiments 2 and 3, wherein the 4-aminobutyrate-2-oxoglutarate transaminase (EC 2.6.1.19) has a SEQ ID NO. 7.

Embodiment 11. The genetically modified host cell of embodiment 1 and the method of embodiments 2 and 3, wherein the 2-aminoethylphosphonate-pyruvate transaminase (EC 2.6.1.37) has a SEQ ID NO. 8.

Embodiment 12. The genetically modified host cell of embodiment 1 and the method of embodiments 2 and 3, wherein the heterologous nucleic acids comprising the recombinant biosynthetic pathway are derived from a Streptomyces bacterium.

Embodiment 13. The genetically modified host cell of embodiment 1 and the method of embodiments 2 and 3, wherein the heterologous nucleic acids comprising the pathway are derived from a Streptomyces rubellomurinus.

Embodiment 14. The genetically modified host cell of embodiment 1 and the method of embodiments 2 and 3, wherein at least two of phosphoenolpyruvate mutase (EC 5.4.2.9), 2-phosphonomethylmalate synthase (EC 2.3.3.19), aconitate hydratase (EC 4.2.1.3), isocitrate dehydrogenase NAD⁺ (EC 1.1.1.41), isocitrate dehydrogenase NADP+ (EC 1.1.1.42), an isozyme of isocitrate dehydrogenase NAD⁺ (EC 1.1.1.41), an isozyme of isocitrate dehydrogenase NADP+ (EC 1.1.1.42), and a transaminase are operatively associated to comprise the biosynthetic pathway.

Embodiment 15. The genetically modified host cell of embodiment 1 and the method of embodiments 2 and 3, wherein the phosphoenolpyruvate mutase (EC 5.4.2.9) is operatively associated with 2-phosphonomethylmalate synthase (EC 2.3.3.19) to convert phosphonopyruvate into 2-phosphonomethylmalate.

Embodiment 16. The genetically modified host cell of embodiment 1 and the method of embodiments 2 and 3, wherein the 2-phosphonomethylmalate synthase (EC 2.3.3.19) is operatively associated with the aconitate hydratase (EC 4.2.1.3) to convert 2-phosphonomethylmalate into 3-phosphonomethylmalate.

Embodiment 17. The genetically modified host cell of embodiment 1 and the method of embodiments 2 and 3, wherein the aconitate hydratase (EC 4.2.1.3) is operatively associated with the isocitrate dehydrogenase NAD⁺ (EC 1.1.1.41), the isocitrate dehydrogenase NADP+(EC 1.1.1.42), the isozyme of the isocitrate dehydrogenase NAD⁺ (EC 1.1.1.41), and the isozyme of the isocitrate dehydrogenase NADP+ (EC 1.1.1.42) to convert 3-phosphonomethylmalate into 2-oxo-4-phosphonobutyrate.

Embodiment 18. The genetically modified host cell of embodiment 1 and the method of embodiments 2 and 3, wherein the isocitrate dehydrogenase NAD⁺ (EC 1.1.1.41), the isocitrate dehydrogenase NADP+ (EC 1.1.1.42), the isozyme of the isocitrate dehydrogenase NAD⁺ (EC 1.1.1.41), and the isozyme of the isocitrate dehydrogenase NADP+ (EC 1.1.1.42) are operatively associated with the transaminase to convert 2-oxo-4-phosphonobutyrate into nh-pSer.

Embodiment 19. The genetically modified host cell of embodiment 1 and the method of embodiments 2 and 3, wherein the phosphoenolpyruvate mutase (EC 5.4.2.9) has a SEQ ID NO. 1.

Embodiment 20. The genetically modified host cell of embodiment 1 and the method of embodiments 2 and 3, wherein the 2-phosphonomethylmalate synthase (EC 2.3.3.19) has a SEQ ID NO. 2.

Embodiment 21. The genetically modified host cell of embodiment 1 and the method of embodiments 2 and 3, wherein the aconitate hydratase (EC 4.2.1.3) has a SEQ ID NO. 3.

Embodiment 22. The genetically modified host cell of embodiment 1 and the method of embodiments 2 and 3, wherein the isocitrate dehydrogenase NAD⁺ (EC 1.1.1.41) and the isocitrate dehydrogenase NADP+ (EC 1.1.1.42) have a SEQ ID NO. 4.

Embodiment 23. The genetically modified host cell of embodiment 1 and the method of embodiments 2 and 3, wherein the isozyme of the isocitrate dehydrogenase NAD⁺ (EC 1.1.1.41) and the isozyme of the isocitrate dehydrogenase NADP+ (EC 1.1.1.42) have a SEQ ID NO. 5.

Embodiment 24. The genetically modified host cell of embodiment 1 and the method of embodiments 2 and 3, wherein the biosynthetic pathway requires the isocitrate dehydrogenase NAD⁺ (EC 1.1.1.41) and the isocitrate dehydrogenase NADP+ (EC 1.1.1.42).

Embodiment 25. The genetically modified host cell of embodiment 1 and the method of embodiments 2 and 3, wherein the biosynthetic pathway requires the isozyme of the isocitrate dehydrogenase NAD⁺ (EC 1.1.1.41) and the isozyme of the isocitrate dehydrogenase NADP+ (EC 1.1.1.42).

Embodiment 26. The genetically modified host cell of embodiment 1 and the method of embodiments 2 and 3, wherein the tRNA is an orthogonal tRNA that recognizes the UAG amber codon.

Embodiment 27. The genetically modified host cell of embodiment 1 and the method of embodiments 2 and 3, wherein the aaRS is an orthogonal aaRS that preferentially aminoacylates the orthogonal tRNA with the nh-pSer to produce the protein of interest containing at least one nhpSer.

Embodiment 28. The genetically modified host cell of embodiment 1 and the method of embodiments 2 and 3, wherein the expression vector is a plasmid or a virus.

Embodiment 29. The genetically modified host cell of embodiment 1 and the method of embodiments 2 and 3, wherein the genetically modified host cell is a prokaryotic cell or a eukaryotic cell.

Embodiment 30. The genetically modified host cell of embodiment 1 and the method of embodiments 2 and 3, wherein the prokaryotic or eukaryotic host cell is a yeast, a bacterium, an insect cell, a plant cell, or a mammalian cell.

Embodiment 31. The genetically modified host cell of embodiment 1 and the method of embodiments 2 and 3, wherein the yeast is Saccharomyces cerevisiae, Pichia pastoris, Hansenula polymorpha, Yarrowia lipolytica, Arxula adeninivorans, Kluyveromyces lactis, or Schizosaccharomyces pombe.

Embodiment 31. The genetically modified host cell of embodiment 1 and the method of embodiments 2 and 3, wherein the bacterium is Escherichia coli, Bacillus subtilis, or Salmonella typhimuium.

Embodiment 32. The genetically modified host cell of embodiment 1 and the method of embodiments 2 and 3, wherein the insect cell is Spodopterafrugiperdai.

Embodiment 33. The genetically modified host cell of embodiment 1 and the method of embodiments 2 and 3, wherein the mammalian cell is a CHO cell, a COS cell, a HEK cell, or a HeLa cell.

Embodiment 34. The genetically modified host cell of embodiment 1 and the method of embodiments 2 and 3, wherein the protein of interest is an extracellular protein or an intracellular protein.

Embodiment 35. The genetically modified host cell of embodiment 1 and the method of embodiments 2 and 3, wherein the extracellular or intracellular protein has at least one native phosphorylation site.

Embodiment 36. The genetically modified host cell of embodiment 1 and the method of embodiments 2 and 3, wherein the nh-pSer contains a carbon-phosphorus bond.

In this context, the following examples illustrate methods for confirming the synthesis, incorporation, and stability of PermaPhos nhpSer proteins of interest.

### EXAMPLES

### Example 1: PermaPhos Cells Can Incorporate nhpSer Into a Model Protein

In this example, the model protein, super-folder green fluorescent protein (sfGFP), was used to determine whether the PermaPhos cell system could incorporate nhpSer into a single position (1×) or in two positions (2×) as illustrated in FIG. 14A. Briefly, a heterologous nucleic acid encoding either a sfGFP containing an amber codon inserted at a first position, and/or a second sfGFP with an amber codon inserted at a first position and/or a second position were expressed in the PermaPhos cell. Specifically, the host cell is E. coli BL21(DE3) ΔserC. This cell line is a derivative of traditional BL21(DE3) cells (which has within its chromosome a T7 polymerase under the transcriptional control of the lac promoter) that contains the deletion of the serC gene to prevent synthesis of phosphoserine. A second host cell can also include an RF1 deficient expression cell line, B95(DE3) ΔA ΔfabR ΔserC. This cell line will produce PermaPhos nhpSer containing proteins without buildup of truncated peptide. This cell line is a derivative of BL21(DE3) with the following alterations: 95 chromosomal TAG stop codons were mutated to TAA; RF1 was knocked out (ΔA); FabR was knocked out (ΔfabR); SerC was knocked out (ΔserC).

The cells were cultured according to cell culture techniques well known to one with ordinary skill in the art. After the cells reached confluency, the sfGFP containing cells were isolated using well-known affinity techniques that target the affinity tag associated with the sfGFP reporter plasmid. The bar graph compares the yield of the sfGFP-nhpSer protein, wherein the nhpSer was produced either through the biosynthetic pathway or supplementing nhpSer to the culture media. As illustrated, compared to the WT protein, the biosynthetic pathway yielded over 100 mg/per liter of culture of the 1× sfGFP-nhpSer protein and about 50 mg/per liter of culture of the 2× sfGFP-nhpSer protein.

In contrast, supplementing nhpSer to the culture media resulted in negligible sfGFP-nhpSer protein production for either the 1× or 2× nhpSer as compared to the WT protein. Further, to test for accurate incorporation of the phosphate group, purified proteins from these cultures were run on SDS-PAGE and Phos-tag gels. See FIG. 14B. Proteins in SDS-PAGE migrate according to overall size. Proteins in Phos-tag gels migrate slower with the presence of a phosphate group. As illustrated in the SDS-PAGE gel, the nhpSer containing proteins (1× or 2×), whether synthesized with the PermaPhos system or supplemented with nhpSer had a similar molecular weight compared to the pSer containing protein or the WT protein. Additionally, the Phos-tag gel indicates that the nhpSer containing proteins (1× or 2×) produced by the PermaPhos system contained the targeted number of nhpSer moieties as these proteins migrate slower than the WT protein and the pSer (1× or 2×) proteins. FIG. 14C shows that whole protein mass spectrometry confirms faithful incorporation of nhpSer using PermaPhos technology. For comparison, the equivalent proteins with pSer (N150 pSer, D134/N150 pSer) are included to confirm that the approximately 2 Da difference in mass between nhpSer and pSer results from the oxygen to methylene substitution. See e.g., FIG. 2A.

Therefore, these data show that the PermaPhos cell system can successfully incorporate nhpSer into a model protein, at either 1× or 2× sites, and this cell system yields far more sfGFP-nhpSer per liter of culture compared to similar systems that rely on supplementing nhpSer.

### Example 2: Synthesis of a Specific Phosphatase Inhibitor Using PermaPhos Cells

In this example, a short 27 amino acid peptide containing nhpSer was synthesized in a host cell to determine if the peptide could act as a specific inhibitor of the phosphatase calcineurin (CN). As illustrated in FIG. 15A, the nhpSer containing peptide was expressed as a fusion construct with the small ubiquitin-like modifier (SUMO) attached to its N-terminus and sfGFP attached to its C-terminus; both SUMO and sfGFP can be proteolytically cleaved to yield the isolated 27 amino acid peptide. The isolated peptide-sfGFP fusion was run on an SDS-PAGE gel and a Phos-tag gel to determine if nhpSer was successfully incorporated into the peptide. The host cell is E. coli BL21(DE3) ΔserC. This cell line is a derivative of traditional BL21(DE3) cells (which has within its chromosome a T7 polymerase under the transcriptional control of the lac promoter) that contains the deletion of the serC gene to prevent synthesis of phosphoserine. A second host cell can also include an RF1 deficient expression cell line, B95(DE3) ΔA ΔfabR ΔserC. This cell line will produce PermaPhos nhpSer containing proteins without buildup of truncated peptide. This cell line is a derivative of BL21(DE3) with the following alterations: 95 chromosomal TAG stop codons were mutated to TAA; RF1 was knocked out (ΔA); FabR was knocked out (ΔfabR); SerC was knocked out (ΔserC).

As illustrated in FIG. 15B (top gel CN(−)), the nhpSer incorporated protein displayed similar molecule weight to the WT and pSer containing proteins as determined by SDS-PAGE. Further, the nhpSer containing protein migrated slower than the WT and pSer containing proteins on Phos-tag gels indicating the presence of a phosphate group. See FIG. 15B (bottom gel CN(−)). All together, these data suggest that nhpSer was successfully incorporated into the protein.

To determine whether the nhpSer containing protein was resistant to dephosphorylation by CN, the gels were exposed to CN. If nhpSer containing protein is not resistant to dephosphorylation by CN, it should behave similarly to the pSer containing proteins-pSer proteins are not resistant to dephosphorylation. As indicated by the Phos-tag gel, the nhpSer containing protein is resistant to dephosphorylation by CN, as it migrates similar to the nhpSer containing protein in the absence of CN. See FIG. 15B (bottom gel CN(+)).

To determine the purity of the nhpSer containing protein following a large-scale purification process, proteins isolated from culture media were run on an SDS-PAGE gel and a Phos-tag gel as previously described for FIG. 15B. Purification of the sfGFP protein followed well know purification techniques. Purification yielded approximately 15 mg of purified peptide fused to sfGFP per liter of culture media, which is approximately 25% of the WT yield. See FIG. 15C. From the 15 mg of purified peptide fused to sfGFP, approximately 2 mg of the 27-mer peptide-nhpSer can be purified per liter of culture media.

### Example 3: PermaPhos Synthesized nhpSer Can Regulate Phosphoserine-Dependent

### Complexes.

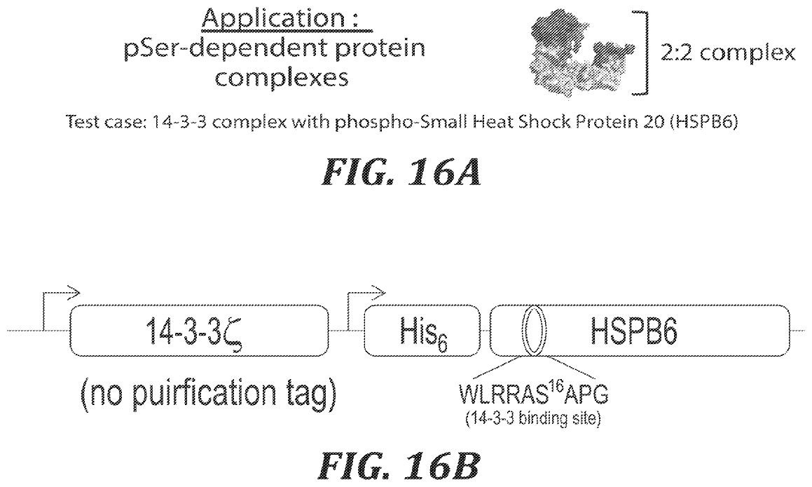

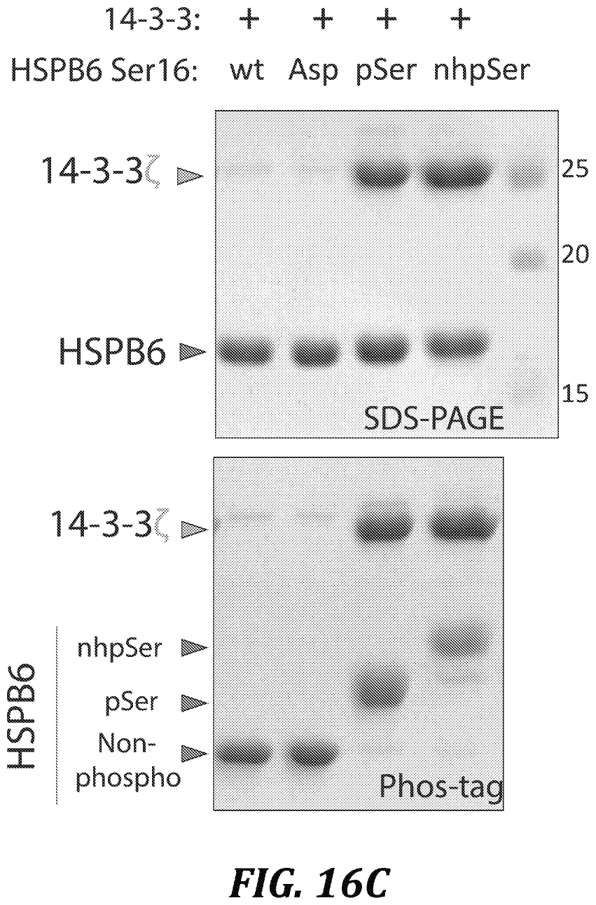

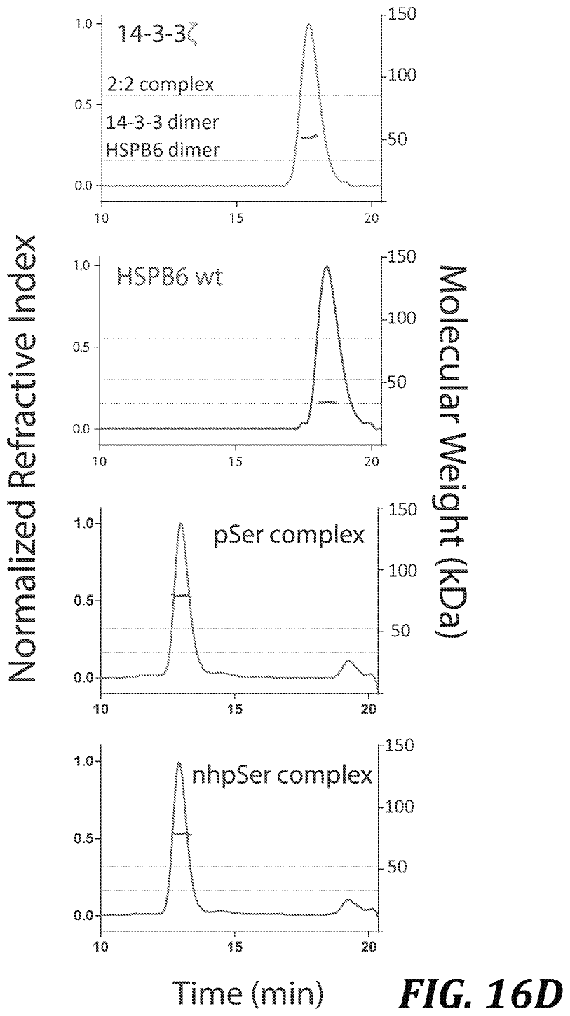

In this example, a stable phospho-dependent protein complex was created to determine whether a PermaPhos synthesized nhpSer containing protein can act as a functional mimic of the native pSer containing protein as illustrated in FIG. 16. The stable phospho-dependent protein complex, as shown in FIG. 16A, comprises an untagged 14-3-3 protein—a protein known to specifically bind to pSer containing proteins- and the phosphorylated heat shock protein 20 (HSPB6), containing a purification tag (FIG. 16B). The host cell is E. coli BL21(DE3) ΔserC. This cell line is a derivative of traditional BL21(DE3) cells (which has within its chromosome a T7 polymerase under the transcriptional control of the lac promoter) that contains the deletion of the serC gene to prevent synthesis of phosphoserine. A second host cell can also include an RF1 deficient expression cell line, B95(DE3) ΔA ΔfabR ΔserC. This cell line will produce PermaPhos nhpSer containing proteins without buildup of truncated peptide. This cell line is a derivative of BL21(DE3) with the following alterations: 95 chromosomal TAG stop codons were mutated to TAA; RF1 was knocked out (ΔA); FabR was knocked out (ΔfabR); SerC was knocked out (ΔserC). Therefore, if nhpSer can function as a stable mimic of pSer, this will enable the 14-3-3 protein to bind to and form a stable complex with HSPB6, which is determined upon the co-purification of the untagged 14-3-3 protein with the purification tagged HSPB6, as shown in FIG. 16C.

As illustrated in FIG. 16C (SDS-PAGE), 14-3-3 co-purifies with the HSPB6 protein when either pSer or PermaPhos nhpSer are incorporated at amino acid residue 16 of the HSPB6 protein. The WT and Asp lanes indicate that 14-3-3 does not co-purify with the HSPB6 protein when a serine or aspartate is incorporated at amino acid residue 16 of the HSPB6 protein. Purification of the phospho-dependent protein complex yields 15 mg of nhpSer complex per liter of culture. Additionally, nhpSer is successfully incorporated into the complex as illustrated by Phos-tag gels.

As another test to confirm the incorporation of PermaPhos-synthesized nhpSer can be used to generate phosphorylation dependent complexes is illustrated in FIG. 16D. FIG. 16D shows that by size-exclusion chromatography coupled to multi-angle light scattering (SEC-MALS) measurements, the 14-3-3/pSer-HSPB6 complex is identical to the PermaPhos-synthesized 14-3-3/nhpSer HSPB6 complex at least by molecular weight and stoichiometry. Therefore, these data suggest that the PermaPhos-generated nhpSer containing protein forms tight, specific complexes between HSPB6 and the pSer specific binding protein 14-3-3, demonstrating that nhpSer is a functional mimic of pSer.

In a related example, the ability of nhpSer to act as a functional mimic of pSer by monomerizing 14-3-3 was tested. 14-3-3 dimerization can be regulated by phosphorylation of a serine residue at the dimer interface, e.g., serine residue 58 (Ser58). Therefore, if nhpSer can function as a functional mimic of pSer, expressing nhpSer at position Ser58-phosphorylation of this position controls 14-3-3 dimerization-will allow for 14-3-3 to exist as a monomer rather than a dimer as determined by size exclusion liquid chromatography coupled to multi-angle light scattering (SEC-MALS).

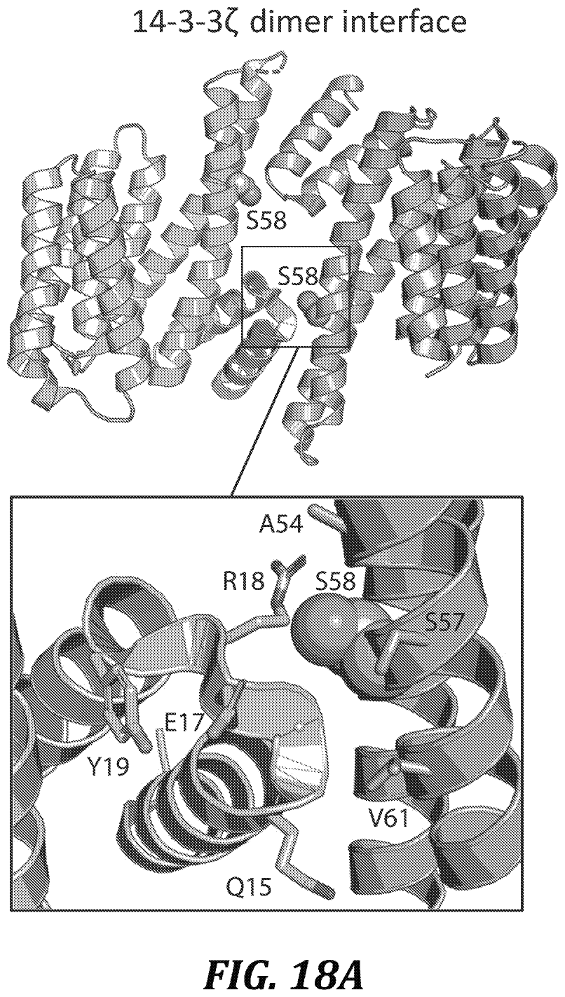

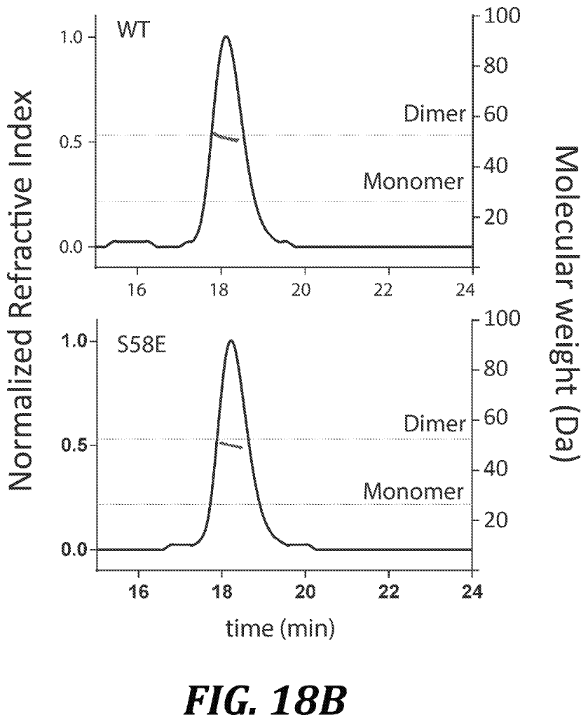

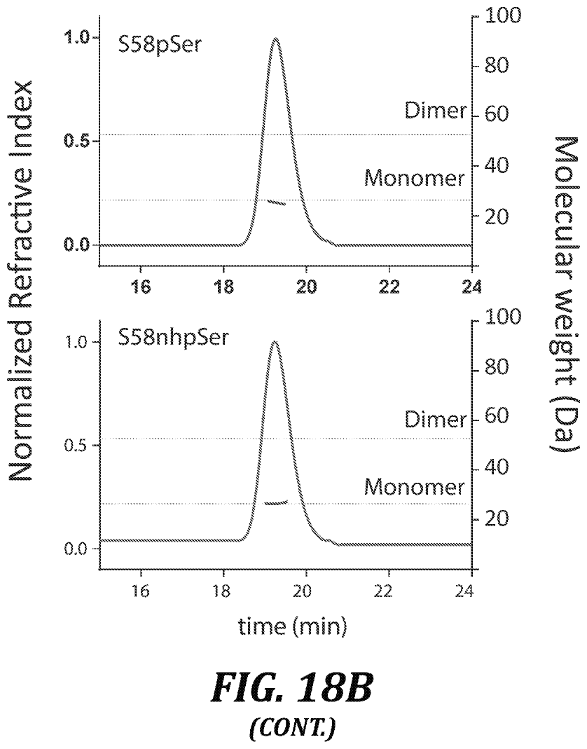

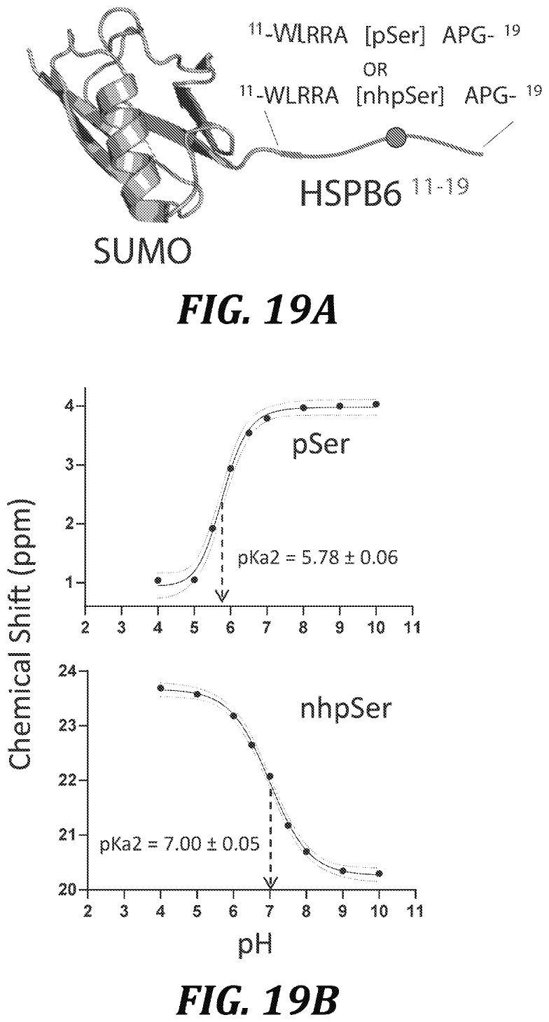

FIG. 18A illustrates a representation of 14-3-3ζ dimer interface. The inset of FIG. 18A illustrates a different orientation of the dimer interface at position Ser58. FIG. 18B illustrates the results from expressing 14-3-3ζ with serine (WT), glutamate, pSer, and nhpSer at residue 58. Measurements from SEC-MALS confirmed that 14-3-3ζ is a dimer. See FIG. 18B (WT). S58E phosphomimetic also maintained its dimeric status (FIG. 18B S58E). Additionally, demonstrating that 14-3-3 dimerization can be regulated by phosphorylation of serine (Ser58), pSer and nhpSer at site 58 fully monomerizes 14-3-3. See FIG. 18B (S58pSer) and (S58nhpSer). These data confirm that nhpSer is a functional mimic of pSer to monomerize 14-3-3, while the traditional glutamate cannot serve as a functional mimic of pSer.

### Example 4: PermaPhos Synthesized nhpSer Can Promote Enzyme Catalytic Activity

In this example, PermaPhos-synthesized nhpSer can function to promote enzyme catalytic activity. The enzyme activity of the enzyme glycogen synthase kinase-3beta (GSK3) is promoted by binding to substrates that contain pSer. Binding to pSer substrates primes GSK3, which brings the catalytic residues of GSK3 into proper position to phosphorylate a serine or threonine residue-located four residues upstream from the phosphorylated residues of the substrate. Therefore, if PermaPhos-synthesized nhpSer is a functional mimic of pSer, the PermaPhos-synthesized nhpSer will prime GSK3 activity for phosphorylation of the upstream serine and threonine residues.

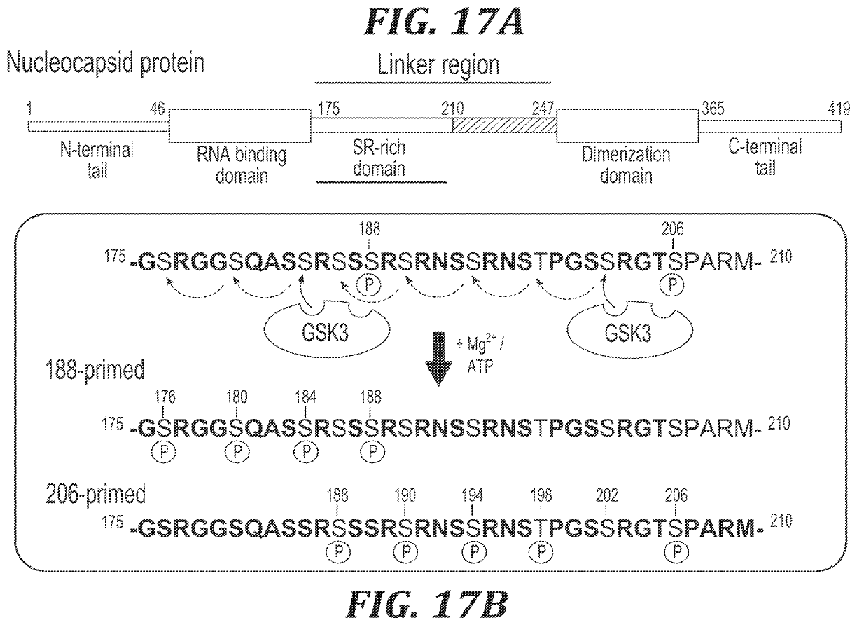

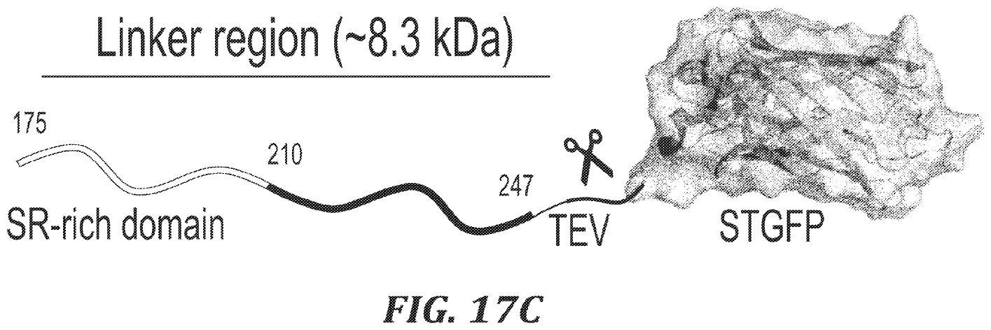

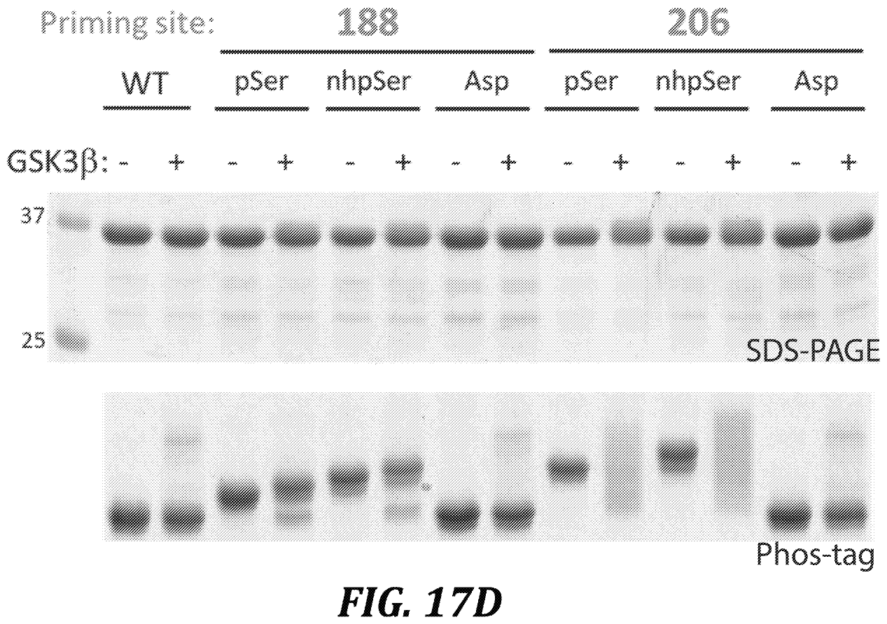

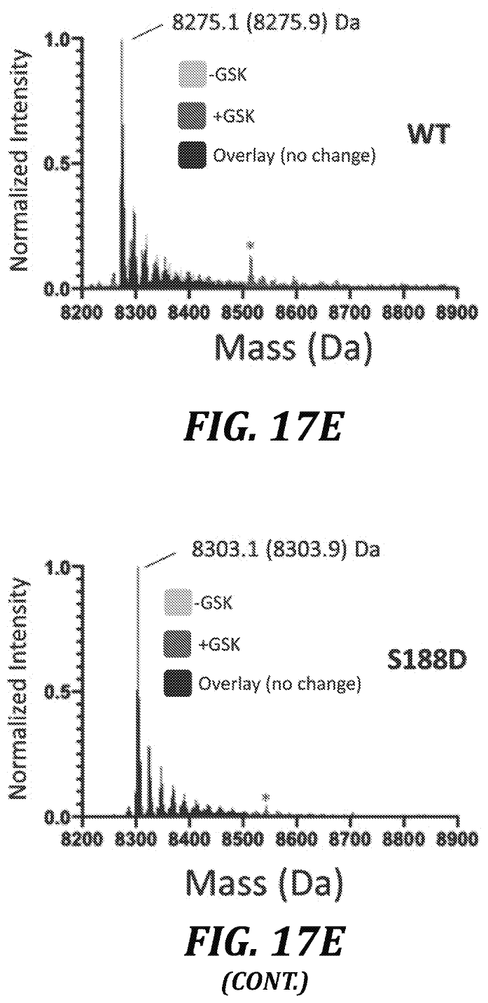

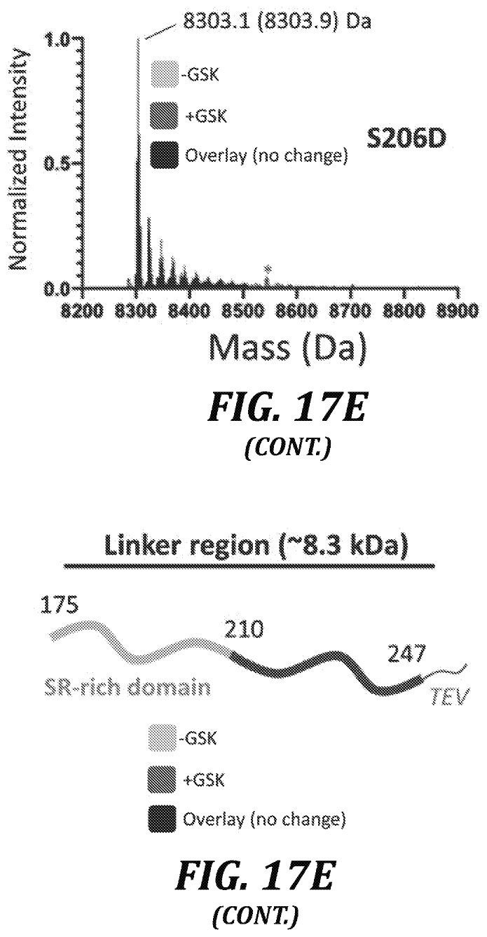

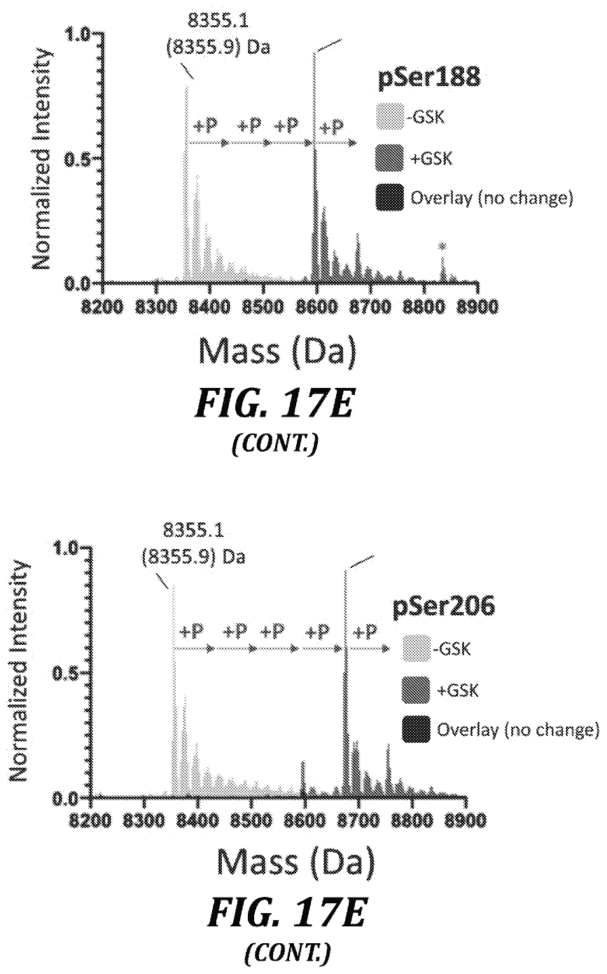

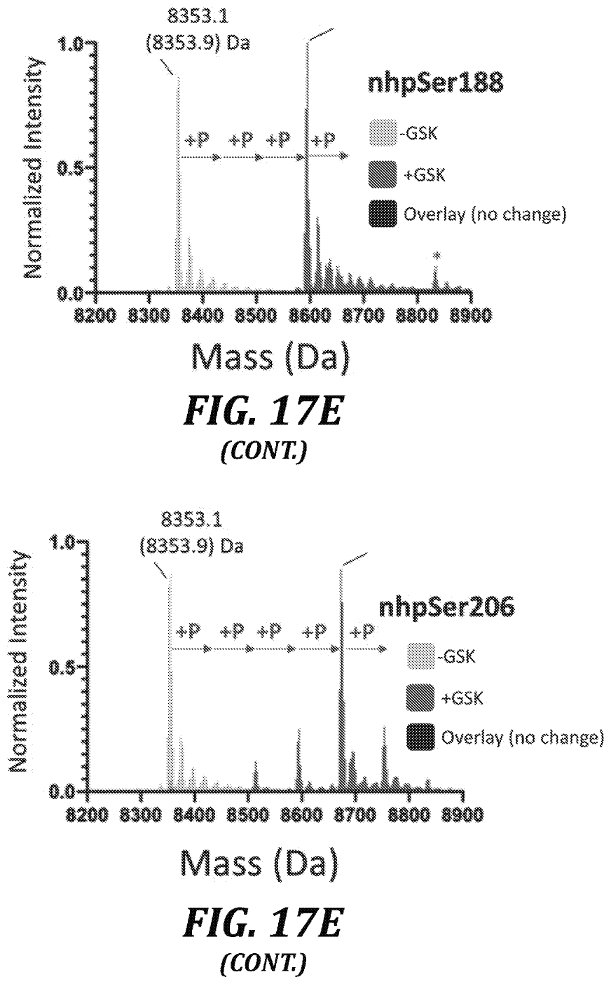

To determine if PermaPhos-synthesized nhpSer can serve as a functional mimic of pSer to prime GSK3 activity, the linker region of the Covid-19 nucleocapsid protein (N-protein, residues 175-247) was expressed in a host cell with either Ser (WT), Asp, pSer or PermaPhos-synthesized nhpSer at sites 188 and 206-two sites that are known to prime GSK3 activity. See FIGS. 17A-17C. The Phos-tag gel in FIG. 17D and whole-protein mass spectrometry analyses in FIG. 17E illustrate that GSK3 cannot phosphorylate the non-phosphorylated WT and Asp mutants of the nucleocapsid protein (FIG. 17D, WT and Asp lanes and FIG. 17E, WT and S188D/S206D panels). Similar to the pSer at amino acid positions 188 and 206, the PermaPhos-synthesized nhpSer at position 188 and 206 can prime GSK3 for phosphorylation of the N-protein. See FIG. 17D, pSer and nhpSer lanes and FIG. 17E, pSer188, nhpSer188, pSer206 and nhpSer206 panels). Therefore, these data show that nhpSer containing Covid-19 nucleocapsid proteins incorporated PermaPhos-synthesized nhpSer, which can promote pSer-dependent GSK3 activity. In this example, the host cell is E. coli BL21(DE3) ΔserC. This cell line is a derivative of traditional BL21(DE3) cells (which has within its chromosome a T7 polymerase under the transcriptional control of the lac promoter) that contains the deletion of the serC gene to prevent synthesis of phosphoserine. A second host cell can also include an RF1 deficient expression cell line, B95(DE3) ΔA ΔfabR ΔserC. This cell line will produce PermaPhos nhpSer containing proteins without buildup of truncated peptide. This cell line is a derivative of BL21(DE3) with the following alterations: 95 chromosomal TAG stop codons were mutated to TAA; RF1 was knocked out (ΔA); FabR was knocked out (ΔfabR); SerC was knocked out (ΔserC).

### Example 5: Creating a Prokaryotic PermaPhos Organism

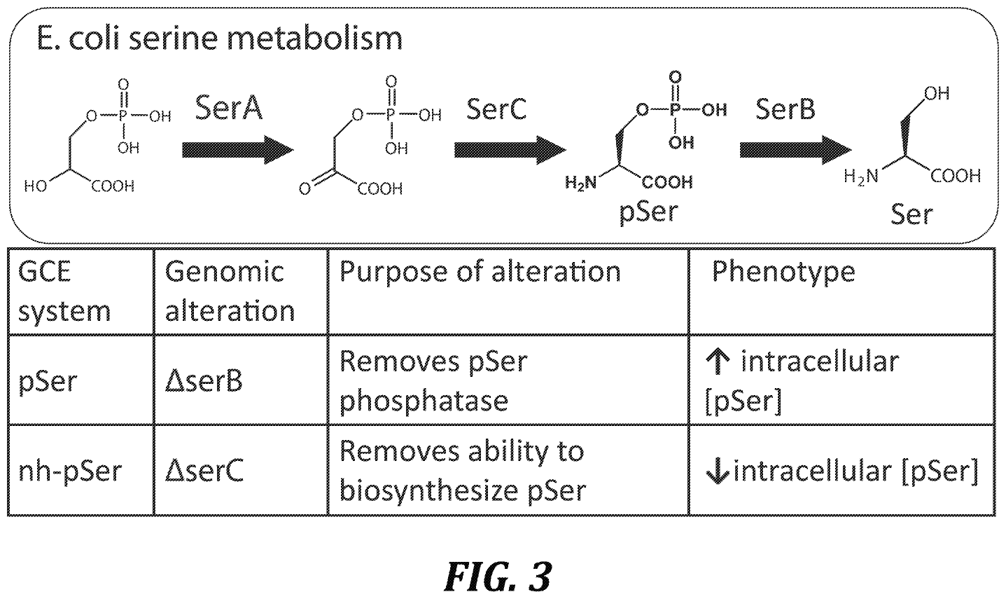

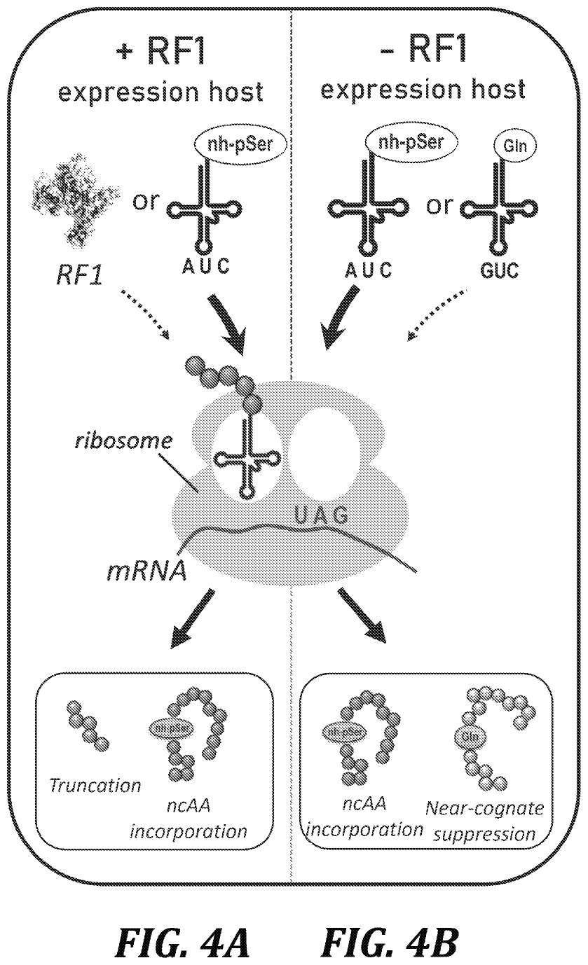

In this example, strains of E. coli used to develop the FrbABCDE biosynthetic pathway express RF1, so truncated peptide builds up in the cell as illustrated in FIG. 4. The PermaPhos prokaryotic proto-type organisms express (i) homogenously modified nhpSer and (ii) do so in the absence of truncated peptide. To create this PermaPhos prokaryotic proto-type organism, the serC gene required for nhpSer incorporation (see FIG. 3) was knocked out of the existing RF1-deficient strain B-95(DE3) ΔA ΔfabR using λ-red recombineering protocols. The B-95(DE3) strain was chosen because it is a RF1-knockout strain derived from BL21(DE3) with minimal endogenous amber suppressor capacity and it grows at nearly the same rate as BL21(DE3), in a wide range of temperatures and media, including minimal and auto-induction media. The creation of the B-95(DE3) ΔA ΔfabR ΔserC strain was confirmed by analytical PCR and genomic sequencing.

### Example 6: Creating a Eukaryotic PermaPhos Organism

Analogous to how nhpSer biosynthesis and incorporation into proteins was accomplished in E. coli, nhpSer biosynthesis and incorporation into proteins can be achieved in eukaryotic cells. This can be done by expressing FrbA, FrbB, FrbC, FrbD and FrbE proteins (or their orthologs) as well as a transaminase in a eukaryotic cell to convert phosphoenolpyruvate into nhpSer. Alongside, the phosphoserine amino-acyl tRNA synthetase, phosphoserine tRNA_{CUA}, phosphoserine compatible elongation factor 1 alpha, and a serine phosphatase will be co-expressed to eliminate competing intracellular phosphoserine and allow for the faithful translational incorporation of nhpSer. With the nhpSer biosynthesis and translational installation components being together inside the same eukaryotic cell, nhpSer can be installed into a target protein that is co-expressed from a gene containing a TAG amber stop codon at the intended site of incorporation.

Process for creating an autonomous eukaryotic cell able to synthesize permanently phosphorylated proteins.

Stage 1: Assembly of the Frb cluster into a eukaryotic expression vector. The five Frb genes (A, B, C, D and E) plus a transaminase (e.g. GabT from E. coli or the PalB-like transaminase from Agrobacterium tumefaciens strain CFBP6625) and a free serine phosphatase (e.g. SerB from E. coli) will be assembled into a single vector. Gene transcription can be controlled by a variety of available eukaryotic promoters, including but not limited to CMV, EF1alpha and UBC. Gene transcription can also be controlled by inducible or light-activated promoters for regulated synthesis of permanently phosphorylated proteins. Genes will be combined into a single poly-cistronic transcript that are separated by e.g. P2A or IRES elements so that each protein is translated as an individual peptide. Or, each gene can be transcribed under the control of its own promoter and translated independent of the other genes. Libraries of plasmids can be generated in which each gene is transcribed at a different level, which after screening will allow for identification of the optimal activity of each protein to maximize metabolic flux through the pathway. These plasmids will be transfected into eukaryotic cells such as HEK293T, and metabolomic analyses will identify whether nhpSer is being synthesized and to what level.

Stage 2: Delete phosphoserine amino transferase (PSAT) from the genome of the expression host. Using CRISPR or other established strategies, PSAT will be deleted from the genome of e.g HEK293T cells. This, in combination with expression of a free serine phosphatase, will prevent biosynthesis of phosphoserine and eliminate its competition with nhpSer for incorporation in the target protein.

Stage 3: Generating eukaryotic nhpSer translational incorporation machinery plasmids. Next, a plasmid expressing phosphoserine amino-acyl tRNA synthetase (SepRS), phosphoserine tRNA_{CUA} (Sep-tRNA), and a phosphoserine compatible elongation factor 1 alpha (Sep-EF1a) will be constructed. The SepRS and Sep-EF1a will be expressed from Ubc or EF1A promoters and the Sep-tRNA expressed from U6 and/or HI promoters. The number of Sep-tRNA gene copies can vary between 4 and 32 for optimal Sep-tRNA expression levels. A second plasmid will be created that expresses the protein of interest containing a TAG codon (for directing nhpSer incorporation) and also expresses between 4 and 32 copies of Sep-tRNA.

Stage 4: Creating the eukaryotic PermaPhos cell. Three plasmids will be co-transfected into e.g. HEK293T cells. The first plasmid contains the nhpSer biosynthetic pathway, the second contains the nhpSer translational installation machinery, and the third plasmid will express the protein of interest (as well as additional copies of Sep-tRNA). Initially, the protein of interest will be a fluorescent reporter protein such as sfGFP-150TAG, which will allow for validation of nhpSer incorporation by fluorescence microscopy. Cells lacking the nhpSer biosynthetic pathway while expressing the nhpSer translational installation machinery and target protein will be used as a negative control to ensure protein production is the result of nhpSer biosynthesis. Expression efficiency will be optimized by altering expression levels of the Frb protein, SepRS, and the Sep-tRNA.

### Example 7: Assess the Stability of nhpSer in Presence of Phosphatases and Eukaryotic Cell Lysates

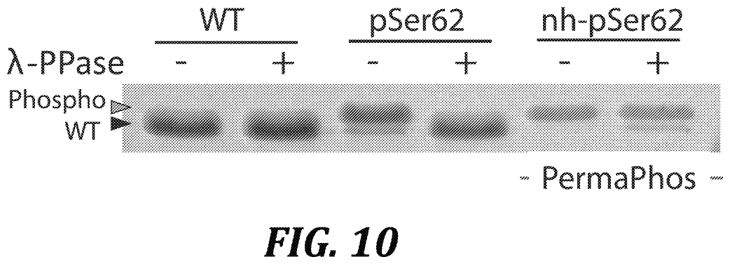

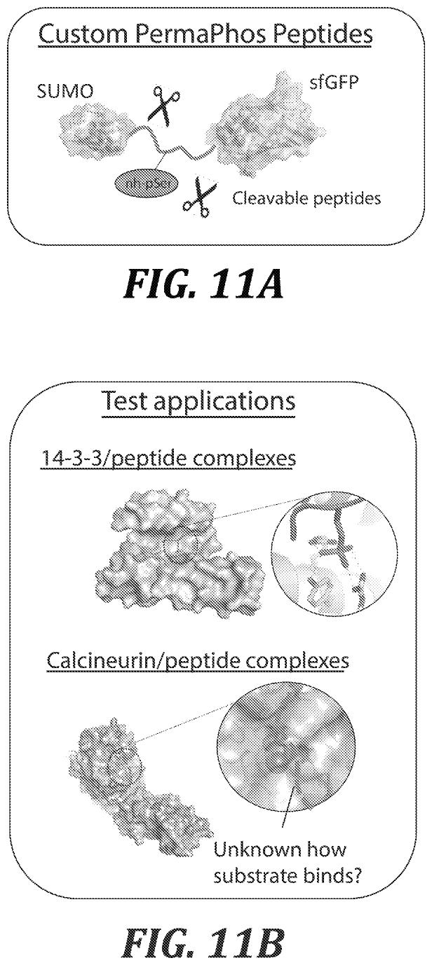

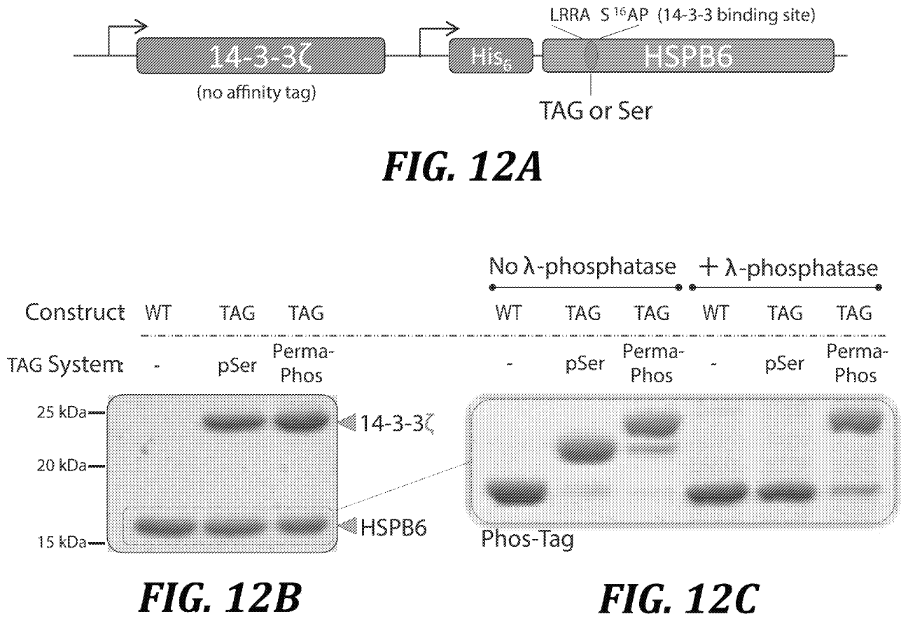

In this example, the stability of biosynthetically produced nhpSer in proteins was evaluated upon exposure to phosphatases. To this end, Bcl-xL was expressed in a host cell with pSer and nhpSer at the biologically relevant site serine residue 62 (Ser62). The nhpSer containing variant produced with the PermaPhos pathway was resistant to λ-phosphatase treatment while the pSer protein was fully hydrolyzed as illustrated in FIG. 10. Similar to sfGFP expression, a small amount (<10%) of the nhpSer protein contains pSer that can be hydrolyzed. Yields of Bcl-xL-nhpSer62 were approximately 1-2 mg per liter of culture, with wild-type approximately 20-fold higher. Perma-Phos nhpSer resistance to calcineurin phosphatase treatment is demonstrated by treating a calcineurin substrate peptide with calcineurin, as shown in FIG. 15B. Lastly, FIG. 12C shows HSPB6 with Perma-Phos nhpSer at residue 16 is resistant to λ-phosphatase treatment as well. In this example, the host cell is E. coli BL21(DE3) ΔserC. This cell line is a derivative of traditional BL21(DE3) cells (which has within its chromosome a T7 polymerase under the transcriptional control of the lac promoter) that contains the deletion of the serC gene to prevent synthesis of phosphoserine. A second host cell can also include an RF1 deficient expression cell line, B95(DE3) ΔA ΔfabR ΔserC. This cell line will produce PermaPhos nhpSer containing proteins without buildup of truncated peptide. This cell line is a derivative of BL21(DE3) with the following alterations: 95 chromosomal TAG stop codons were mutated to TAA; RF1 was knocked out (ΔA); FabR was knocked out (ΔfabR); SerC was knocked out (ΔserC). Similar methods will be used to determine the stability of nhpSer in the presence of eukaryotic cell lysates.

### Example 8: Confirmation of Attenuated Constitutive and Light Controlled Promoters

To begin, variants of the well-established eukaryotic constitutive CMV and EFla promoters with attenuated transcription will be tested using an sfGFP fluorescent reporter in HEK293 cells (FIG. 6). Fluorescence will be quantified using fluorescence-activated cell sorting (FACS). A set of 3 constitutive promoters spanning one order of magnitude of transcriptional values will be targeted. In addition, the light-activated transcription system, the EL222 receptor from E. litoralis, will be validated with sfGFP reporters. Based on the optimized set of Frb components identified in optimization experiments, combinatorial libraries of Frb assemblies will be generated (FIG. 7) in which all promoters are constitutive, and also where one Frb protein is under the control of a light activated promoter (FIG. 13). Library integrity will be confirmed by sequencing and restriction digestion. Genes for each biosynthetic protein will be human codon optimized.

Additionally, to assess expression of permanently phosphorylated sfGFP in HEK cells using constitutive and light-activated Frb biosynthetic pathways, Frb combinatorial libraries will be co-transfected into phosphoserine phosphatase deficient HEK293 cells with a TAG sfGFP-150TAG reporter and the previously published eukaryotic nhpSer GCE machinery (Beranek, V.; Reinkemeier, C. D.; Zhang, M. S.; Liang, A. D.; Kym, G.; Chin, J. W., Genetically Encoded Protein Phosphorylation in Mammalian Cells. Cell Chem Biol 2018, 25 (9), 1067-1074 e5). Cells will be sorted by FACS, and the top expressing hits will be clonally isolated. To rigorously characterize the quality of nhpSer incorporation, sfGFP proteins will be affinity purified from HEK cells and assessed for fidelity by mass spectrometry and Phos-tag gels, following the methods described for the initial characterization of clones 1.8, 2.6, and 4C9 reported in FIG. 8. Clones 1.8, 2.6, and 4C9 are examples of embodiments that are functional. Additional optimization can be carried out to identify more efficient clones. Yields and fidelity of sfGFP-150nhpSer will be directly compared to proteins expressed with exogenously added nhpSer to the culture media (which requires 25 mM nhpSer). Toxicity effects of adding this extracellular nhpSer will be evaluated by standard cell viability assays well known to one of ordinary skill in the art.

To demonstrate control of a signaling pathway, MEK1 will be expressed, either singly or doubly phosphorylated at the serine amino acid residues at sites 218 and 222 (S218 and S222) with nhpSer (FIG. 13). Yields and fidelity of singly and doubly incorporated nhpSer MEK will be evaluated by Phos-Tag gels and Western blots using established phosphospecific MEK antibodies. Time courses of ERK1 activation will be assessed using commercially available α-phospho-ERK1 antibodies. Activation of downstream genes can be assessed by quantitative PCR. Both constitutive and light-activated Frb pathway systems will be tested. Direct comparison with previously published methods in which nhpSer is added to the media to activate MEK1 will be performed.

### Example 9: Characterization of Phosphorylated Monomeric 14-3-3 ζ Pm14-3-3) and Identifying New Pm14-3-3 Client Complexes

14-3-3 is an essential family of dimeric hub proteins that bind to and regulate as many as 2000 different “client” proteins. Formation of these 14-3-3/client complexes depends on the client proteins being phosphorylated at one or more specific serine/threonine sites that reside within specific sequence motifs recognized by 14-3-3. By binding to 14-3-3, client activity, localization or ability to interact with other partner proteins can be tightly controlled in a phosphorylation dependent manner. Because many client proteins are involved in regulating cell cycle, apoptosis, cell migration and cell proliferation signaling systems, and their dysregulation is well correlated with diseases, many 14-3-3/client complexes are of high interest for therapeutic intervention.

14-3-3 proteins themselves are known to be phosphorylated at several sites, but the exact functional changes to 14-3-3 that occur upon phosphorylation are not well understood. One well-known site of phosphorylation on 14-3-3 is at Ser58 (C isoform numbering, SEQ ID NO: 12, Table 3), a residue located at the 14-3-3 dimer interface. The inventors recently confirmed phosphorylation at Ser58 causes dimeric 14-3-3 to dissociate into individual monomers, but it remained unclear how this would change client binding and regulation. The inventors set out to better understand the function of phosphorylated monomeric 14-3-3 (pm14-3-3) and discover new pm14-3-3/client complexes that could be important as novel therapeutic targets.

Revealing the pm14-3-3 interactome.

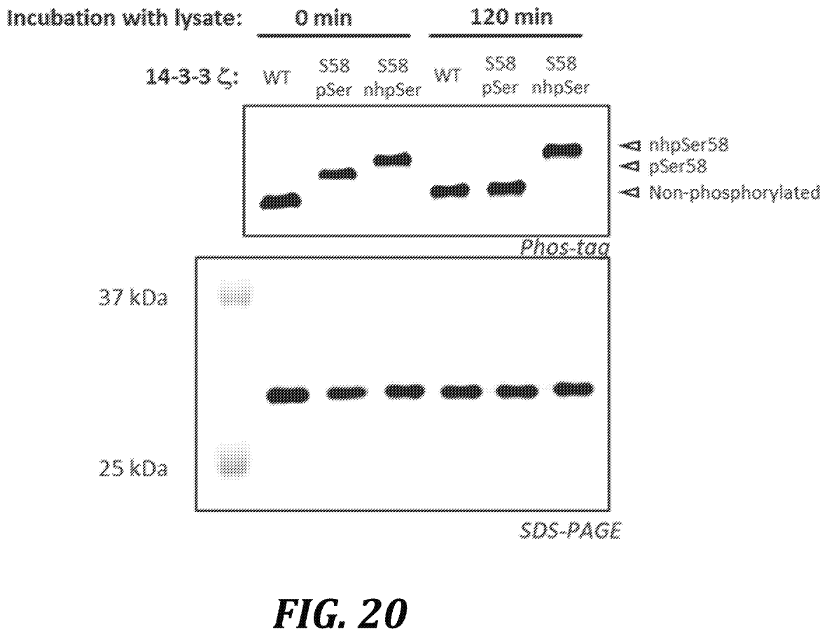

Wild-type 14-3-3ζ and 14-3-3ζ pSer58 were expressed in E. coli, and PermaPhos technology was used to express 14-3-3ζ nhpSer58, all with a FLAG tag at their N-terminus, and then all were purified to homogeneity. After incubation in soluble HEK293 cell lysates, 14-3-3ζ pSer58 was completely hydrolyzed to 14-3-3ζ WT even in the presence of a potent phosphatase inhibitor, indicating that 14-3-3ζ pSer58 reverted back to a dimer (FIG. 20). On the other hand, 14-3-3ζ nhpSer58 was impervious to hydrolysis by phosphatases. These results show that 14-3-3ζ nhpSer58 made via PermaPhos, which mimics the same monomeric structure of authentic 14-3-3ζ pSer58, can be a useful tool to study the function of pm14-3-3 in cellular-like environments.

Next, wild type 14-3-3ζ, 14-3-3ζ pSer58, and 14-3-3ζ nhpSer58 were incubated in HEK293 soluble cell lysates for 2 h, allowing them to bind to any endogenous interacting protein partners present in the lysate. The 14-3-3 proteins and any interacting protein partners were then retrieved via immobilization on a resin, and the interacting protein partners and their relative abundances were characterized by proteomic mass spectrometry. These data revealed the interactome of pm14-3-3, which when compared to wild-type/dimeric 14-3-3 begins to help uncover the function of pm14-3-3.

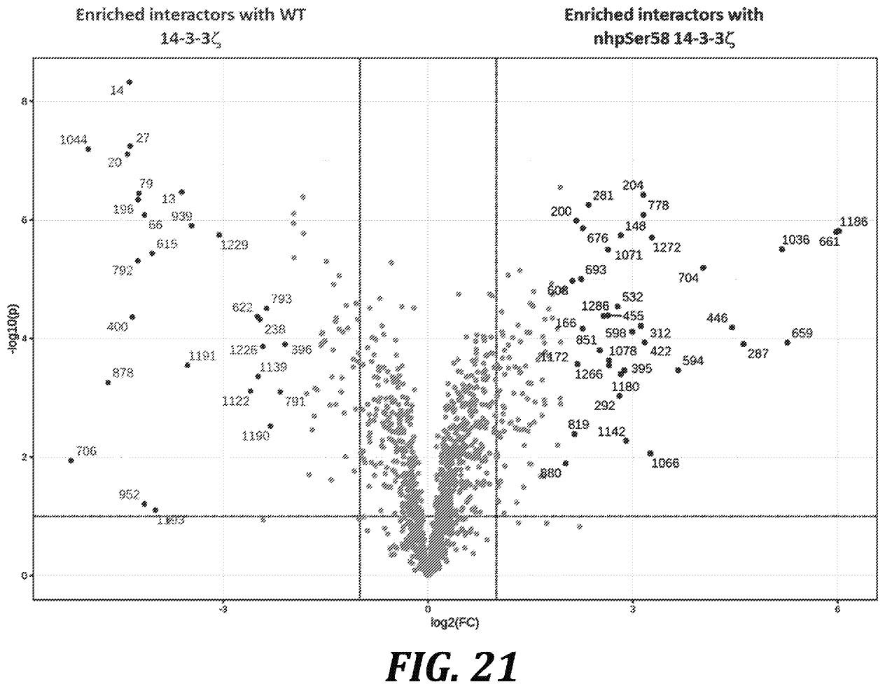

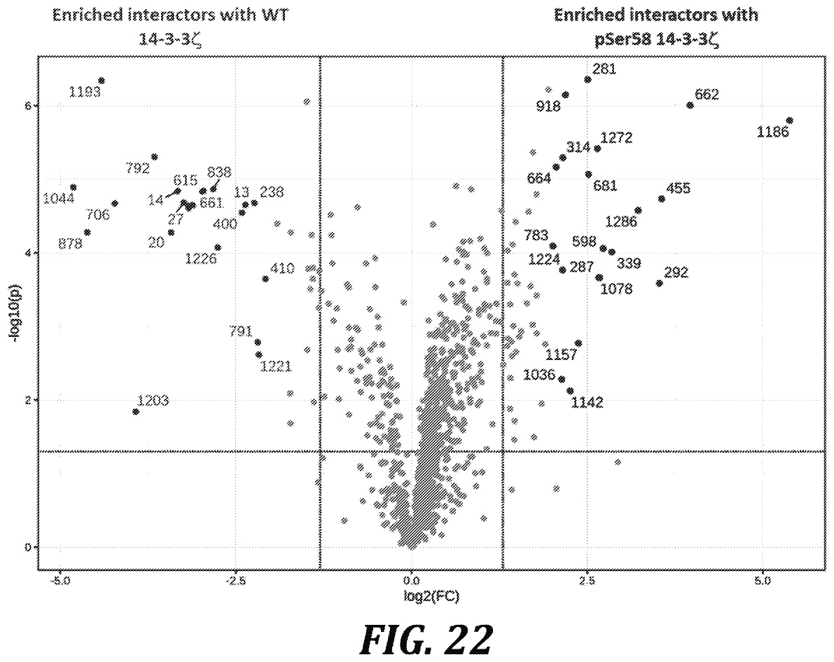

Several important observations were made in these analyses. First, the identity and abundance of most interacting client proteins were identical between WT 14-3-3 and pm14-3-3 ζ, which is unexpected since prior precedent suggests monomeric 14-3-3 is not able to bind client proteins (FIG. 21). That monomeric 14-3-3 ζ can still bind most clients stably means phosphorylation at Ser58 and subsequent monomerization is not a client release switch, but a mechanism to adjust client function by changing the structural dynamics of 14-3-3/client complexes.

Second, while most interacting client proteins are similar between WT and pm14-3-3 ζ, there exist pools of client proteins wherein their binding to 14-3-3 is specific to the pm14-3-3 form (Tables 1 and 2).

TABLE 1
Identity of enriched proteins shown in the Volcano plot analysis of
FIG. 21. Client proteins 28-63 (i.e., numbering in first column) bind
at least 4-fold more specifically to 14-3-3 ζ monomerized by nhpSer58
compared to those interacting with 14-3-3 WT. Client proteins 1-27
(i.e., numbering in first column) bind at least 4-fold more specifically
to 14-3-3 WT compared to those interacting with 14-3-3 ζ
monomerized by nhpSer58.

| | Client | | | |
|---|---|---|---|---|
| | protein | | | |
| | number | Description | FC | Log2(FC) |
| 1 | 706 | 2-amino-3-ketobutyrate | 0.026666 | −5.2289 |
| | | coenzyme A ligase, | | |
| | | mitochondrial | | |
| | | [OS = Homo sapiens] | | |
| 2 | 1044 | 39S ribosomal protein | 0.031771 | −4.9761 |
| | | L14, mitochondrial | | |
| | | [OS = Homo sapiens] | | |
| 3 | 878 | 60S ribosomal protein L32 | 0.038778 | −4.6886 |
| | | [OS = Homo sapiens] | | |
| 4 | 20 | 14-3-3 protein theta | 0.047292 | −4.4023 |
| | | [OS = Homo sapiens] | | |
| 5 | 14 | 14-3-3 protein epsilon | 0.048194 | −4.375 |
| | | [OS = Homo sapiens] | | |
| 6 | 27 | 14-3-3 protein beta/alpha | 0.048615 | −4.3625 |
| | | [OS = Homo sapiens] | | |
| 7 | 400 | Serine/arginine-rich | 0.049613 | −4.3331 |
| | | splicing factor 10 | | |
| | | [OS = Homo sapiens] | | |
| 8 | 792 | Atherin | 0.052409 | −4.254 |
| | | [OS = Homo sapiens] | | |
| 9 | 196 | 14-3-3 protein sigma | 0.052632 | −4.2479 |
| | | [OS = Homo sapiens] | | |
| 10 | 79 | 14-3-3 protein gamma | 0.053128 | −4.2344 |
| | | [OS = Homo sapiens] | | |
| 11 | 952 | A-kinase anchor protein 9 | 0.056079 | −4.1564 |
| | | [OS = Homo sapiens] | | |
| 12 | 66 | 14-3-3 protein eta | 0.056124 | −4.1552 |
| | | [OS = Homo sapiens] | | |
| 13 | 615 | Cell division cycle | 0.060748 | −4.041 |
| | | 5-like protein | | |
| | | [OS = Homo sapiens] | | |
| 14 | 1193 | Pre-mRNA-splicing | 0.0628 | −3.9931 |
| | | factor SLU7 | | |
| | | [OS = Homo sapiens] | | |
| 15 | 13 | 14-3-3 protein zeta/delta | 0.082053 | −3.6073 |
| | | [OS = Homo sapiens] | | |
| 16 | 1191 | V-type proton ATPase | 0.086835 | −3.5256 |
| | | subunit d 1 | | |
| | | [OS = Homo sapiens] | | |
| 17 | 939 | Transformer-2 protein | 0.090641 | −3.4637 |
| | | homolog alpha | | |
| | | [OS = Homo sapiens] | | |
| 18 | 1229 | serrate RNA effector | 0.11997 | −3.0592 |
| | | molecule homolog | | |
| | | [OS = Homo sapiens] | | |
| 19 | 1122 | Neurofibromin | 0.16486 | −2.6007 |
| | | [OS = Homo sapiens] | | |
| 20 | 622 | GTPase-activating protein and | 0.17693 | −2.4987 |
| | | VPS9 domain-containing | | |
| | | protein 1 | | |
| | | [OS = Homo sapiens] | | |
| 21 | 1139 | NCK-interacting protein | 0.17781 | −2.4916 |
| | | with SH3 domain | | |
| | | [OS = Homo sapiens] | | |
| 22 | 238 | Polyadenylate-binding protein 1 | 0.18165 | −2.4608 |
| | | [OS = Homo sapiens] | | |
| 23 | 1226 | Histone deacetylase complex | 0.18699 | −2.419 |
| | | subunit SAP18 | | |
| | | [OS = Homo sapiens] | | |
| 24 | 793 | transformer-2 protein | 0.19402 | −2.3657 |
| | | homolog beta | | |
| | | [OS = Homo sapiens] | | |
| 25 | 1190 | WD and tetratricopeptide | 0.20195 | −2.308 |
| | | repeats protein 1 | | |
| | | [OS = Homo sapiens] | | |
| 26 | 791 | Cytochrome c oxidase subunit | 0.22312 | −2.1641 |
| | | 5A, mitochondrial | | |
| | | [OS = Homo sapiens] | | |
| 27 | 396 | E3 ubiquitin-protein | 0.23405 | −2.0951 |
| | | ligase MYCBP2 | | |
| | | [OS = Homo sapiens] | | |
| 28 | 880 | Acyl-CoA dehydrogenase | 4.0402 | 2.0144 |
| | | family member 10 | | |
| | | [OS = Homo sapiens] | | |
| 29 | 608 | striatin | 4.3344 | 2.1158 |
| | | [OS = Homo sapiens] | | |
| 30 | 819 | Cyclin-dependent kinase | 4.4215 | 2.1445 |
| | | 2-associated protein 1 | | |
| | | [OS = Homo sapiens] | | |
| 31 | 200 | Centrosomal protein of 55 kDa | 4.5004 | 2.17 |
| | | [OS = Homo sapiens] | | |
| 32 | 1172 | F-box only protein 50 | 4.5542 | 2.1872 |
| | | [OS = Homo sapiens] | | |
| 33 | 693 | Protein S100-A9 | 4.7328 | 2.2427 |
| | | [OS = Homo sapiens] | | |
| 34 | 166 | Junction plakoglobin | 4.8018 | 2.2636 |
| | | [OS = Homo sapiens] | | |
| 35 | 676 | SRA stem-loop-interacting | 4.816 | 2.2678 |
| | | RNA-binding | | |
| | | protein, mitochondrial | | |
| | | [OS = Homo sapiens] | | |
| 36 | 281 | exosome complex exonuclease | 5.094 | 2.3488 |
| | | RRP44 | | |
| | | [OS = Homo sapiens] | | |
| 37 | 851 | 28S ribosomal protein S27, | 5.7153 | 2.5148 |
| | | mitochondrial | | |
| | | [OS = Homo sapiens] | | |
| 38 | 1286 | nuclear pore complex | 5.9321 | 2.5685 |
| | | protein Nup98-Nup96 | | |
| | | [OS = Homo sapiens] | | |
| 39 | 1071 | Pre-mRNA-splicing factor | 6.2094 | 2.6345 |
| | | ATP-dependent RNA | | |
| | | helicase DHX15 | | |
| | | [OS = Homo sapiens] | | |
| 40 | 455 | Arginase-1 | 6.2224 | 2.6375 |
| | | [OS = Homo sapiens] | | |
| 41 | 1078 | Translin-associated protein X | 6.2845 | 2.6518 |
| | | [OS = Homo sapiens] | | |
| 42 | 1266 | Apoptosis-stimulating | 6.2857 | 2.6521 |
| | | of p53 protein 2 | | |
| | | [OS = Homo sapiens] | | |
| 43 | 532 | DNA methyltransferase | 6.8586 | 2.7779 |
| | | 1-associated protein 1 | | |
| | | [OS = Homo sapiens] | | |
| 44 | 292 | Keratin, type II cytoskeletal 78 | 6.9769 | 2.8026 |
| | | [OS = Homo sapiens] | | |
| 45 | 1180 | opioid growth factor receptor | 7.0638 | 2.8205 |
| | | [OS = Homo sapiens] | | |
| 46 | 148 | Desmoglein-1 | 7.0768 | 2.8231 |
| | | [OS = Homo sapiens] | | |
| 47 | 395 | HAUS augmin-like | 7.3429 | 2.8763 |
| | | complex subunit 5 | | |
| | | [OS = Homo sapiens] | | |
| 48 | 1142 | Platelet-activating factor | 7.4687 | 2.9009 |
| | | acetylhydrolase IB | | |
| | | subunit alpha | | |
| | | [OS = Homo sapiens] | | |
| 49 | 598 | Exportin-1 | 7.9477 | 2.9905 |
| | | [OS = Homo sapiens] | | |
| 50 | 312 | Cleavage stimulation | 8.6769 | 3.1172 |
| | | factor subunit 3 | | |
| | | [OS = Homo sapiens] | | |
| 51 | 204 | Keratin, type I cytoskeletal 13 | 8.8924 | 3.1526 |
| | | [OS = Homo sapiens] | | |
| 52 | 778 | Protein cereblon | 8.9144 | 3.1561 |
| | | [OS = Homo sapiens] | | |
| 53 | 422 | SAGA-associated factor 29 | 9.0162 | 3.1725 |
| | | [OS = Homo sapiens] | | |
| 54 | 1066 | Ketosamine-3-kinase | 9.5575 | 3.2566 |
| | | [OS = Homo sapiens] | | |
| 55 | 1272 | BRCA2-interacting | 9.6947 | 3.2772 |
| | | transcriptional | | |
| | | repressor EMSY | | |
| | | [OS = Homo sapiens] | | |
| 56 | 594 | Talin-1 | 12.661 | 3.6623 |
| | | [OS = Homo sapiens] | | |
| 57 | 704 | Intraflagellar transport | 16.347 | 4.031 |
| | | protein 22 homolog | | |
| | | [OS = Homo sapiens] | | |
| 58 | 446 | Intraflagellar transport | 21.9 | 4.4529 |
| | | protein 81 homolog | | |
| | | [OS = Homo sapiens] | | |
| 59 | 287 | Annexin A2 | 24.594 | 4.6202 |
| | | [OS = Homo sapiens] | | |
| 60 | 1036 | Serpin B4 | 36.335 | 5.1833 |
| | | [OS = Homo sapiens] | | |
| 61 | 659 | Protein disulfide-isomerase A6 | 38.372 | 5.262 |
| | | [OS = Homo sapiens] | | |
| 62 | 661 | Melanoma-associated | 63.052 | 5.9785 |
| | | antigen D2 | | |
| | | [OS = Homo sapiens] | | |
| 63 | 1186 | Protein S100-A7 | 64.777 | 6.0174 |
| | | [OS = Homo sapiens] | | |

To the inventors' knowledge, these client proteins represent never-before-known protein complexes that could be important therapeutic targets particularly given their involvement in regulating essential cellular processes.

TABLE 2
Identity of proteins that were detected as unique
interactors with 14-3-3 ζ monomerized by nhpSer58.
Description
galectin-7
[OS = Homo sapiens]
Nuclear distribution protein nudE-like 1
[OS = Homo sapiens]
Serpin B12
[OS = Homo sapiens]
Keratin, type II cytoskeletal 80
[OS = Homo sapiens]
Isoform 10 of Tropomyosin alpha-1 chain
[OS = Homo sapiens]
Protein dpy-30 homolog
[OS = Homo sapiens]
Plakophilin-1
[OS = Homo sapiens]
Fatty acid-binding protein, epidermal
[OS = Homo sapiens]
Protein-glutamine gamma-glutamyltransferase E
[OS = Homo sapiens]
Zinc-alpha-2-glycoprotein
[OS = Homo sapiens]
Lanosterol synthase
[OS = Homo sapiens]
protein S100-A14
[OS = Homo sapiens]
60S ribosomal protein L10
[OS = Homo sapiens]
Calmodulin-like protein 5
[OS = Homo sapiens]
Mitotic-spindle organizing protein 2B
[OS = Homo sapiens]
Utrophin
[OS = Homo sapiens]
Mitochondrial antiviral-signaling protein
[OS = Homo sapiens]
Emerin
[OS = Homo sapiens]
Protein-glutamine gamma-glutamyltransferase K
[OS = Homo sapiens]
Vesicle-associated membrane protein-associated protein B/C
[OS = Homo sapiens]
Isoform 1 of Protein POF1B
[OS = Homo sapiens]
Non-structural maintenance of chromosomes element 4 homolog A
[OS = Homo sapiens]
SH2B adapter protein 1
[OS = Homo sapiens]
Protein FAM114A2
[OS = Homo sapiens]
programmed cell death protein 10
[OS = Homo sapiens]
Myomegalin
[OS = Homo sapiens]

### Outlook

The application data shown here, made possible by PermaPhos technology, reveal the interactome of pm14-3-3 ζ and more broadly the functional effects of phosphorylated 14-3-3 at Ser58. There exist 6 other isoforms of 14-3-3 (β, γ, ε, η, θ, σ) (Table 3, SEQ ID NOs: 9-11 and 13-15), of which four (β, γ, ε and η) (Table 3, SEQ ID NOs: 9-11 and 13) can also be monomerized by phosphorylation at the equivalent position as Ser58 of isoform ζ .

In other embodiments, any amino acid located at the dimer interface can be phosphorylated to monomerize a dimeric 14-3-3 isoform ζ e.g., β, γ, ε, ζ, η, θ, σ). In other embodiments, the dimeric 14-3-3βisomer can be monomerized by phosphorylation (e.g., nhpSer) at amino acid position 60 (Ser60) in the amino acid sequence as set forth in SEQ ID NO: 9. See e.g., highlighted S in Table 3, SEQ ID NO: 9. In some embodiments, the dimeric 14-3-3γ isomer can be monomerized by phosphorylation (e.g., nhpSer) at amino acid position 59 (Ser59) in the amino acid sequence as set forth in SEQ ID NO: 10. See e.g., highlighted S in Table 3, SEQ ID NO: 10. In some embodiments, the dimeric 14-3-3εisomer can be monomerized by phosphorylation (e.g., nhpSer) at amino acid position 59 (Ser59) in the amino acid sequence as set forth in SEQ ID NO: 11. See e.g., highlighted S in Table 3 SEQ ID NO: 11. In some embodiments, the dimeric 14-3-3ζ can be monomerized by phosphorylation (e.g., nhpSer) at amino acid position 58 (Ser58) in the amino acid sequence as set forth in SEQ ID NO: 12. See e.g., highlighted S in Table 3, SEQ ID NO: 12. In some embodiments, the dimeric 14-3-3η isoform can be monomerized by phosphorylation (e.g., nhpSer) at amino acid position 59 (Ser59) in the amino acid sequence as set forth in SEQ ID NO: 13. See e.g., highlighted S in Table 3, SEQ ID NO: 13. In some embodiments, the dimeric 14-3-30 isoform can be monomerized by phosphorylation (e.g., nhpSer) at amino acid position 58 (Ala58) in the amino acid sequence as set forth in SEQ ID NO: 14. See e.g., highlighted A in Table 3, SEQ ID NO: 14. In some embodiments, the dimeric 14-3-36 can be monomerized by phosphorylation (e.g., nhpSer) at amino acid position 58 (Ala58) in the amino acid sequence as set forth in SEQ ID NO: 15. See e.g., highlighted A in Table 3, SEQ ID NO: 15.

14-3-3 isoforms have binding selectivity toward different clients, and so by extension their respective phosphorylated monomeric forms likely bind to a different pool of clients. The same approach as described here with 14-3-3ζ nhpSer58 can be applied to identify the isoform specific interactomes, and obtain a more complete picture of 14-3-3 regulation by monomerization.

Further, 14-3-3 proteins can be phosphorylated at sites other than Ser58 and these modifications are anticipated to have other (though still unknown) effects on 14-3-3 function that are different from the effects of monomerization. With PermaPhos, the same approach can be used to reveal the unique interactomes for the other phosphorylated forms of 14-3-3, and for all seven isoforms. PermaPhos will allow for identification of many new interactomes, signaling systems, and 14-3-3/client complexes that could be important players in human disease and therapeutic development.

While illustrative embodiments have been illustrated and described, it will be appreciated that various changes can be made therein without departing from the spirit and scope of the invention.

TABLE 3
Amino acids sequences

| | | SEQ ID |
|---|---|---|
| Protein | Sequence | NO |
| FrbD enzyme | MTKRTMLRRAIARGGMSIAAGVHDGLSARIAQDAGFDVLWASGLGISAA | 1 |
| (phosphoenol- | HAVPDDSILTMTEFLEAARVMNDSTDLPVLADCDTGFGDERNVARMVER | |
| pyruvate | YERAGIAGVCIEDKVFPKRNSLGDGAQDQETIEGFAAKLAAAKKAQQTD | |
| mutase, | DFVVVARIETFIAGGVKEDAIARADAYVAAGADAILIHSKSRDGLEILD | |
| EC 5.4.2.9) | FGRSWQRKDVPLVAVPTTYPAVTADDLHEAGFSLAIYANQALRASITSM | |
| | RESLRQITHDRSSLRIEPKVAKLSEVFELQR | |
| FrbC enzyme | MRNDLVLEDTTLRDGEQTPGVAFSKETKTAILNALIEAGVTSIEIGIPA | 2 |
| (2-phosphono- | MGGEELDFIKSVVDRQDEARLVVWHRGVREDVERSLDLGFTSVHVGLPT | |
| methylmalate | SAGHLKASVRKDRTWLLATARDMVKMAKDRGAFVSISAEDIARTEISFL | |
| synthase, | QEYAGVVAEAGADRLRLSDTVGLLGPEAYGERVAAVLSAADIDVQCHAH | |
| EC 2.3.3.19) | NDFGLATANTLAGLKAGARYFHVTVNAIGERAGMADLAQVVVALKKLYD | |
| | RDLGIDLTKLKKVSRLVAEAAGHQVLPWQPITGDNVFAHESGIHANGMF | |
| | RDTSSFEPFPPEHVGGERRYVLGKHSGRALVAWALEQEGITPREELLPH | |
| | CLEEVRALSIRIGGAVSHEQLVEIYNKAAA | |
| FrbA enzyme | MRDLLRDLRLPDRNARYVSLAAAEEMGLVGISSAPRSLRVLVENTLRAA | 3 |
| (aconitate | AAAKAGSTGDSLRQAVLSANSLIAAATANGPVSEVEFRPTRLVLQDHSG | |
| hydratase, | IPVLADLASLRSIMADRGLDPRLARPALPVDLVVDHSVEVASWARPDAL | |
| EC 4.2.1.3) | SLNMRREYELNGERYRFLRWAQQEISGLRVVPPGRGIVHQMHLEHLARV | |
| | VLPDGDGLIAPDTVLGTDSHTPMVGALGVLGWGVGGIEAETVLLGHSVS | |
| | LLSPQVIGVRLTGELPVGSTATDLALTMTEFLRNVGVVGRFVEFFGPGV | |
| | CALSVPDRATLSNMAPEYGCTVAFFPVDDTTLRYLELTGRGAEEIRLVE | |
| | AYCKEQGLFAGADASADPEFARVLDFDLSAVEPSAAGPRRPQDRLPLGT | |
| | VPASFPEPSGAALAGAAEGRRELADGAVAIAAITSCTNTANPHSMVTAG | |
| | LVARKAVQRGLAVPSWVKTSLAPGSRAVVHYLEAAGLLPDLEKLGFSVA | |
| | GFGCTTCIGNSGGLAPEAEERARTEGVRLAAVLSGNRNFEGRIHPDVAA | |
| | AYLASPALVVAYALAGTVLTDLTARPVGTDPDGAEVFLADLWPTPEEID | |
| | AAVGLVAREHFASEKESLFTGGPEWTSLEHPSGPVYDWPAESDYLLRSP | |
| | FFDGLTHAPLSDLSGARVLVLTGDSTTTDHISPAGAIGADSPAGRYLRA | |
| | LGVEPALFNSYGCRRGNHEVLVRGTFANPKFRNLLTPDVQGSSTLHVPT | |
| | GERMSVHEAALAYAAEQVPVVVVGGRDYGFGSSRDWAAKGPALLGVRAV | |
| | LAKSFERIHRSNLIGMGIVPLEFLPDQDAGTLGLTGHEALDVIGLDGLV | |
| | PRGTVTVRARSAEGEPVAEWRMLARVDTAGELEYVRRGGFLRSVASDLL | |
| | DAEA | |
| FrbB enzyme | MTVSQYPDVAVRSDSPRPEASVLPAALELLESAGLTANWRIVAGQAPDG | 4 |
| (isocitrate | DGGRKPEAGLLPPFAEPGASAYGLARAARRAWGGTFSVIERRGPLVRSD | |
| dehydrogenase | GTVNVLLFRYELDMSDTGMEFESNSGEQLALVKTAAELGPREIPEQAAL | |
| NAD⁺, | GIQALAPDRVSEFVRTAVGMCRRLDVDDLVVAHKGNSIKHTDGLFALTA | |
| EC 1.1.1.41 and | AETLESLAGAAGIAWRSLIIDHMAAELVRNPLDYRAILTHSSFADLLAA | |
| isocitrate | ALAGQMTTDQLGVCTTYDPAGRPLFGHDPALDTSAPDADPERWRVRRLA | |
| dehydrogenase | STLAAGAALAEHLRLPPAGRALRRAAAELVRRDGGPIGWDQAVTTARKS | |
| NADP⁺, | LRAEGPARS | |
| EC 1.1.1.42) | | |
| FrbE enzyme | MRKHTVTLIAGDGSGPELVAAARTVLDTAAGSAGFHLDWDVRPAGQGAL | 5 |
| (isocitrate | RSVGELLPASTLDSIRRNGVALKGPTTTPIGVGHRSVNITIRRELDLYA | |
| dehydrogenase | CVRPVRVWPGAGAVDSGIDVVVVRENTEDVYSGLEYPADSDGAAVVSAT | |
| NAD⁺, | LSEFGHADPGIAFSLKPITPGGSRRIVAFALDHARKNGRRKVEVGADVY | |
| EC 1.1.1.41 and | RYPRTEGLFFEAARELGPEYPDIVTATVPVTVLCDSLVRDPADRDVLVL | |
| isocitrate | PNLYGDVISDVTAALVGGLGMAPGANIGDRTAVFEATHGSAPEFAGSNR | |
| dehydrogenase | LNPTALILSGCMLLDHLGEAPTAARIRAAVRSVLEEGRSVTFDLLPPDR | |
| NADP⁺, | AARAVTTTAYAEAVIERLRDIG | |
| EC 1.1.1.42) | | |
| serine-pyruvate | MLKSMPWSSGPVACDPPADGPLKDGRLRSDSPRDRPETGPYERDVPVRT | 6 |
| transaminase | SQSTPFARIGRDPEESTAVTHPFLDLAPLTAAHFAAIEQRVARLLSTEQ | |
| (EC 2.6.1.51) | DVVITQGEALLPLEGCIRGAARPGTTALNVITGPYGQTFGNWLRDCGAT | |
| | VIDLAVPFHTAVTAAQIDEALTEHPEIDFVSLVHAEAATGNTNPVAEIG | |
| | EVVRAHGALFMLDAVASVGAEPLLPDAWGVDLCVIGAQKAMGGPAGVSA | |
| | VSVSERAWQRMAANPQAPRRSYLSLLDWKERWIDGGRKALLHAPAQLEM | |
| | LALEACLERIEAEGLGALMTRHATAAAATRAGALALGGGLEPYVYEARD | |
| | AAPVATTLRTPPGVDASEVVARALAADPVLPLVAGGGALSKEMIRVNHY | |
| | GADATRGVVHSALAALGGALAESGLTVNLEAARRAVTDAWS | |
| 4-aminobutyrate- | MNSNKELMQRRSQAIPRGVGQIHPIFADRAENCRVWDVEGREYLDFAGG | 7 |
| 2-oxoglutarate | IAVLNTGHLHPKVVAAVEAQLKKLSHTCFQVLAYEPYLELCEIMNQKVP | |
| transaminase | GDFAKKTLLVTTGSEAVENAVKIARAATKRSGTIAFSGAYHGRTHYTLA | |
| | LTGKVNPYSAGMGLMPGHVYRALYPCPLHGISEDDAIASIHRIFKNDAA | |
| | PEDIAAIVIEPVQGEGGFYASSPAFMQRLRALCDEHGIMLIADEVQSGA | |
| | GRTGTLFAMEQMGVAPDLTTFAKSIAGGFPLAGVTGRAEVMDAVAPGGL | |
| | GGTYAGNPIACVAALEVLKVFEQENLLQKANDLGQKLKDGLLAIAEKHP | |
| | EIGDVRGLGAMIAIELFEDGDHNKPDAKLTAEIVARARDKGLILLSCGP | |
| | YYNVLRILVPLTIEDAQIRQGLEIISQCFDEAKQ | |
| 2-aminoethyl- | MTSRNYLLLTPGPLTTSRTVKEAMLFDSCTWDDDYNIGVVEQIRQQLTA | 8 |
| phosphonate- | LATASEGYTSVLLQGSGSYAVEAVLGSALGPQDKVLIVSNGAYGARMVE | |
| pyruvate | MAGLMGIAHHAYDCGEVARPDVQAIDAILNADPTISHIAMVHSETTTGM | |
| transaminase | LNPIDEVGALAHRYGKTYIVDAMSSFGGIPMDIAALHIDYLISSANKCI | |
| | QGVPGFAFVIAREQKLAACKGHSRSLSLDLYAQWRCMEDNHGKWRFTSP | |
| | THTVLAFAQALKELAKEGGVAARHQRYQQNQRSLVAGMRALGENTLLDD | |
| | ELHSPIITAFYSPEDPQYRFSEFYRRLKEQGFVIYPGKVSQSDCFRIGN | |
| | IGEVYAADITALLTAIRTAMYWTK | |
| 14-3-3 Beta | MTMDKSELVQKAKLAEQAERYDDMAAAMKAVTEQGHELSNEERNLLSVA | 9 |
| (β) | YKNVVGARRSSWRVISSIEQKTERNEKKQQMGKEYREKIEAELQDICND | |
| | VLELLDKYLIPNATQPESKVFYLKMKGDYFRYLSEVASGDNKQTTVSNS | |
| | QQAYQEAFEISKKEMQPTHPIRLGLALNFSVFYYEILNSPEKACSLAKT | |
| | AFDEAIAELDTLNEESYKDSTLIMQLLRDNLTLWTSENQGDEGDAGEGE | |
| | N | |
| 14-3-3 Gamma | MVDREQLVQKARLAEQAERYDDMAAAMKNVTELNEPLSNEERNLLSVAY | 10 |
| (γ) | KNVVGARRSSWRVISSIEQKTSADGNEKKIEMVRAYREKIEKELEAVCQ | |
| | DVLSLLDNYLIKNCSETQYESKVFYLKMKGDYYRYLAEVATGEKRATVV | |
| | ESSEKAYSEAHEISKEHMQPTHPIRLGLALNYSVFYYEIQNAPEQACHL | |
| | AKTAFDDAIAELDTLNEDSYKDSTLIMQLLRDNLTLWTSDQQDDDGGEG | |
| | NN | |
| 14-3-3 Epsilon | MDDREDLVYQAKLAEQAERYDEMVESMKKVAGMDVELTVEERNLLSVAY | 11 |
| (ε) | KNVIGARRASWRIISSIEQKEENKGGEDKLKMIREYRQMVETELKLICC | |
| | DILDVLDKHLIPAANTGESKVFYYKMKGDYHRYLAEFATGNDRKEAAEN | |
| | SLVAYKAASDIAMTELPPTHPIRLGLALNFSVFYYEILNSPDRACRLAK | |
| | AAFDDAIAELDTLSEESYKDSTLIMQLLRDNLTLWTSDMQGDGEEQNKE | |
| | ALQDVEDENQ | |
| 14-3-3 Zeta | MDKNELVQKAKLAEQAERYDDMAACMKSVTEQGAELSNEERNLLSVAYK | 12 |
| (ζ) | NVVGARRSSWRVVSSIEQKTEGAEKKQQMAREYREKIETELRDICNDVL | |
| | SLLEKFLIPNASQAESKVFYLKMKGDYYRYLAEVAAGDDKKGIVDQSQQ | |
| | AYQEAFEISKKEMQPTHPIRLGLALNFSVFYYEILNSPEKACSLAKTAF | |
| | DEAIAELDTLSEESYKDSTLIMQLLRDNLTLWTSDTQGDEAEAGEGGEN | |
| 14-3-3 Eta | MGDREQLLQRARLAEQAERYDDMASAMKAVTELNEPLSNEDRNLLSVAY | 13 |
| (η) | KNVVGARRSSWRVISSIEQKTMADGNEKKLEKVKAYREKIEKELETVCN | |
| | DVLSLLDKFLIKNCNDFQYESKVFYLKMKGDYYRYLAEVASGEKKNSVV | |
| | EASEAAYKEAFEISKEQMQPTHPIRLGLALNFSVFYYEIQNAPEQACLL | |
| | AKQAFDDAIAELDTLNEDSYKDSTLIMQLLRDNLTLWTSDQQDEEAGEG | |
| | N | |
| 14-3-3 Theta | MEKTELIQKAKLAEQAERYDDMATCMKAVTEQGAELSNEERNLLSVAYK | 14 |
| (θ) | NVVGGRRSAWRVISSIEQKTDTSDKKLQLIKDYREKVESELRSICTTVL | |
| | ELLDKYLIANATNPESKVFYLKMKGDYFRYLAEVACGDDRKQTIDNSQG | |
| | AYQEAFDISKKEMQPTHPIRLGLALNFSVFYYEILNNPELACTLAKTAF | |
| | DEAIAELDTLNEDSYKDSTLIMQLLRDNLTLWTSDSAGEECDAAEGAEN | |
| 14-3-3 Sigma | MERASLIQKAKLAEQAERYEDMAAFMKGAVEKGEELSCEERNLLSVAYK | 15 |
| (σ) | NVVGGQRAAWRVLSSIEQKSNEEGSEEKGPEVREYREKVETELQGVCDT | |
| | VLGLLDSHLIKEAGDAESRVFYLKMKGDYYRYLAEVATGDDKKRIIDSA | |
| | RSAYQEAMDISKKEMPPTNPIRLGLALNFSVFHYEIANSPEEAISLAKT | |
| | TFDEAMADLHTLSEDSYKDSTLIMQLLRDNLTLWTADNAGEEGGEAPQE | |
| | PQS | |

The embodiments of the invention in which an exclusive property or privilege is claimed are defined as follows:

1. A method for producing or expressing a protein of interest comprising a non-hydrolyzable phosphoserine (nhpSer), the method comprising:
culturing a genetically modified host cell comprising at least one expression vector that can express the protein of interest comprising the nhpSer, wherein the genetically modified host cell comprises a recombinant biosynthetic pathway comprising:
at least one heterologous nucleic acid encoding a phosphoenolpyruvate mutase (EC 5.4.2.9);
at least one heterologous nucleic acid encoding a 2-phosphonomethylmalate synthase (EC 2.3.3.19);
at least one heterologous nucleic acid encoding an aconitate hydratase (EC 4.2.1.3); and
at least one heterologous nucleic acid encoding an isocitrate dehydrogenase NAD⁺ (EC 1.1.1.41) and isocitrate dehydrogenase NADP+ (EC 1.1.1.42);
wherein the genetically modified host cell is mutated to produce a non-functional releasing factor 1 (RF1) protein responsible for terminating translation at a UAG amber codon; and
wherein the genetically modified host cell comprises:
at least one heterologous nucleic acid that encodes an aminoacyl tRNA synthetase (aaRS), wherein the aaRS can charge a tRNA with the nhpSer;
at least one heterologous nucleic acid that encodes a tRNA, wherein the tRNA can decode the UAG amber codon;
at least one heterologous nucleic acid encoding the protein of interest wherein the amber codon is inserted at a selected position where the non-hydrolyzable phosphoserine is to be inserted; and
culturing the genetically modified host cell under conditions such that the nucleic acids encoding the enzymes of the pathway are translated and the non-hydrolyzable-phosphoserine is inserted into the protein of interest and the nucleic acid encoding the protein of interest is translated thereby incorporating into the protein of interest the nhpSer at the selected position.

2. The method of claim 1, wherein the recombinant biosynthetic pathway further comprises at least one heterologous nucleic acid encoding an isozyme of the isocitrate dehydrogenase NAD⁺ (EC 1.1.1.41) and an isozyme of the isocitrate dehydrogenase NADP+(EC 1.1.1.42), wherein the isozyme of the isocitrate dehydrogenase NAD⁺ (EC 1.1.1.41) and the isozyme of the isocitrate dehydrogenase NADP+ (EC 1.1.1.42) have a sequence as set forth in SEQ ID NO: 5.

3. The method of claim 1, wherein the recombinant biosynthetic pathway further comprises at least one heterologous nucleic acid encoding a transaminase, wherein the addition of the transaminase to the recombinant biosynthetic pathway improves the efficiency of nhpSer biosynthesis.

4. The method of claim 3, wherein the transaminase is selected from the group of serine-pyruvate transaminase (EC 2.6.1.51), comprising a sequence as set forth in SEQ ID NO: 6; 4-aminobutyrate-2-oxoglutarate transaminase (EC 2.6.1.19), comprising a sequence as set forth in SEQ ID NO: 7; and 2-aminoethylphosphonate-pyruvate transaminase (EC 2.6.1.37), comprising a sequence as set forth in SEQ ID NO: 8.

5. The method of claim 1, wherein the heterologous nucleic acids comprising the recombinant biosynthetic pathway are derived from a Streptomyces bacterium.

6. The method of claim 5, wherein at least two of phosphoenolpyruvate mutase (EC 5.4.2.9), 2-phosphonomethylmalate synthase (EC 2.3.3.19), aconitate hydratase (EC 4.2.1.3), isocitrate dehydrogenase NAD⁺ (EC 1.1.1.41), isocitrate dehydrogenase NADP+ (EC 1.1.1.42), an isozyme of isocitrate dehydrogenase NAD⁺ (EC 1.1.1.41), an isozyme of isocitrate dehydrogenase NADP+ (EC 1.1.1.42), and a transaminase are operatively associated to comprise the biosynthetic pathway.

7. The method of claim 6, wherein the phosphoenolpyruvate mutase (EC 5.4.2.9), comprising a sequence as set forth in SEQ ID NO: 1, is operatively associated with the 2-phosphonomethylmalate synthase (EC 2.3.3.19), comprising a sequence as set forth in SEQ ID NO: 2, to convert phosphonopyruvate into 2-phosphonomethylmalate.

8. The method of claim 6, wherein the 2-phosphonomethylmalate synthase (EC 2.3.3.19) is operatively associated with the aconitate hydratase (EC 4.2.1.3), comprising a sequence as set forth in SEQ ID NO: 3, to convert 2-phosphonomethylmalate into 3-phosphonomethylmalate.

9. The method of claim 6, wherein the aconitate hydratase (EC 4.2.1.3) is operatively associated with the isocitrate dehydrogenase NAD⁺ (EC 1.1.1.41), the isocitrate dehydrogenase NADP+ (EC 1.1.1.42), the isozyme of the isocitrate dehydrogenase NAD⁺ (EC 1.1.1.41), and the isozyme of the isocitrate dehydrogenase NADP+ (EC 1.1.1.42) to convert 3-phosphonomethylmalate into 2-oxo-4-phosphonobutyrate, wherein the isocitrate dehydrogenase NAD⁺ (EC 1.1.1.41) and the isocitrate dehydrogenase NADP+ (EC 1.1.1.42) have a sequence as set forth in SEQ ID NO: 4.

10. The method of claim 6, wherein the isocitrate dehydrogenase NAD⁺ (EC 1.1.1.41), the isocitrate dehydrogenase NADP+ (EC 1.1.1.42), the isozyme of the isocitrate dehydrogenase NAD⁺ (EC 1.1.1.41), and the isozyme of the isocitrate dehydrogenase NADP+(EC 1.1.1.42) are operatively associated with the transaminase to convert 2-oxo-4-phosphonobutyrate into nhpSer.

11. The method of claim 6, wherein the biosynthetic pathway requires the isocitrate dehydrogenase NAD⁺ (EC 1.1.1.41) and the isocitrate dehydrogenase NADP+ (EC 1.1.1.42).

12. The method of claim 6, wherein the biosynthetic pathway requires the isozyme of the isocitrate dehydrogenase NAD⁺ (EC 1.1.1.41) and the isozyme of the isocitrate dehydrogenase NADP+ (EC 1.1.1.42).

13. The method of claim 1, wherein one or more of the heterologous genes of the recombinant biosynthetic pathway are inserted into an expression vector.

14. The method of claim 1, wherein the genetically modified host cell is a prokaryotic cell or a eukaryotic cell.

15. The method of claim 1, wherein the protein of interest is 14-3-3ζ .

16. The method of claim 15, wherein the 14-3-3ζ comprises nhpSer at amino acid position 58 (Ser58) in the sequence as set forth in SEQ ID NO: 12.

17. The method of claim 1, wherein the tRNA is an orthogonal tRNA that recognizes the UAG amber codon.

18. The method of claim 17, wherein the aaRS is an orthogonal aaRS that preferentially aminoacylates the orthogonal tRNA with the nh-pSer to produce the protein of interest containing at least one nh-pSer.

19. A method to identify at least one intracellular protein that stably binds to a monomeric 14-3-3 protein, the method comprising:
producing a 14-3-3 nhpSer protein according to the method of claims 1-18, wherein the nhpSer is expressed at a position to monomerize the dimeric 14-3-3 protein;
incubating the monomeric 14-3-3 nhpSer protein in a soluble lysate for a period of time to allow the monomeric 14-3-3 nhpSer protein to bind to at least one intracellular protein in the soluble lysate to form a 14-3-3-nhpSer complex;
separating the 14-3-3-nhpSer complex from the soluble cell lysate; and
characterizing the 14-3-3-nhpSer complex to identify the stably bound intracellular protein.

20. The method of claim 19, wherein the monomeric 14-3-3 nhpSer protein is a 14-3-3 isoform selected from one of 14-3-3ζ, 14-3-3β, 14-3-3γ, 14-3-3ε, 14-3-3η, 14-3-3θ, or 14-3-3σ.

21. The method of claim 20, wherein the monomeric 14-3-3 isoform is 14-3-3ζ and expressing nhpSer at amino acid position 58 (Ser58) in the sequence as set forth in SEQ ID NO: 12 dissociates dimeric 14-3-3ζ into two 14-3-3ζ monomers.

22. A genetically modified host cell that can produce a protein of interest comprising a non-hydrolyzable phosphoserine (nhpSer), wherein the genetic modification comprises:
a recombinant biosynthetic pathway, wherein the recombinant biosynthetic pathway comprises:
at least one heterologous nucleic acid encoding a phosphoenolpyruvate mutase (EC 5.4.2.9);
at least one heterologous nucleic acid encoding a 2-phosphonomethylmalate synthase (EC 2.3.3.19);
at least one heterologous nucleic acid encoding an aconitate hydratase (EC 4.2.1.3); and
at least one heterologous nucleic acid encoding an isocitrate dehydrogenase NAD⁺ (EC 1.1.1.41) and/or isocitrate dehydrogenase NADP+ (EC 1.1.1.42); and
a recombinant translational system, wherein the recombinant translational system comprises:
at least one heterologous nucleic acid encoding a non-functional releasing factor-1 (RF1);
at least one heterologous nucleic acid encoding an aminoacyl tRNA synthetase (aaRS);
at least one heterologous nucleic acid encoding a tRNA; and
at least one heterologous nucleic acid encoding the protein of interest;
wherein the genetically modified host cell produces an increased amount of the nh-pSer incorporated into the protein of interest compared to host cells which are not genetically modified.

23. The genetically modified host cell of claim 22, wherein at least two of phosphoenolpyruvate mutase (EC 5.4.2.9), 2-phosphonomethylmalate synthase (EC 2.3.3.19), aconitate hydratase (EC 4.2.1.3), isocitrate dehydrogenase NAD⁺ (EC 1.1.1.41), isocitrate dehydrogenase NADP+ (EC 1.1.1.42), an isozyme of isocitrate dehydrogenase NAD⁺ (EC 1.1.1.41), an isozyme of isocitrate dehydrogenase NADP+ (EC 1.1.1.42), and a transaminase are operatively associated to comprise the biosynthetic pathway.


SEQUENCE LISTING (15 sequences)

SEQ ID NO: 1 - protein, 276 aa, Streptomyces rubellomurinus
MTKRTMLRRAIARGGMSIAAGVHDGLSARIAQDAGFDVLWASGLGISAAHAVPDDSILTMTEFLEAARVMNDSTDLPVLADCDTGFGDERNVARMVERYERAGIAGVCIEDKVFPKRNSLGDGAQDQETIEGFAAKLAAAKKAQQTDDFVVVARIETFIAGGVKEDAIARADAYVAAGADAILIHSKSRDGLEILDFGRSWQRKDVPLVAVPTTYPAVTADDLHEAGFSLAIYANQALRASITSMRESLRQITHDRSSLRIEPKVAKLSEVFELQR

SEQ ID NO: 2 - protein, 373 aa, Streptomyces rubellomurinus
MRNDLVLEDTTLRDGEQTPGVAFSKETKTAILNALIEAGVTSIEIGIPAMGGEELDFIKSVVDRQDEARLVVWHRGVREDVERSLDLGFTSVHVGLPTSAGHLKASVRKDRTWLLATARDMVKMAKDRGAFVSISAEDIARTEISFLQEYAGVVAEAGADRLRLSDTVGLLGPEAYGERVAAVLSAADIDVQCHAHNDFGLATANTLAGLKAGARYFHVTVNAIGERAGMADLAQVVVALKKLYDRDLGIDLTKLKKVSRLVAEAAGHQVLPWQPITGDNVFAHESGIHANGMFRDTSSFEPFPPEHVGGERRYVLGKHSGRALVAWALEQEGITPREELLPHCLEEVRALSIRIGGAVSHEQLVEIYNKAAA

SEQ ID NO: 3 - protein, 886 aa, Streptomyces rubellomurinus
MRDLLRDLRLPDRNARYVSLAAAEEMGLVGISSAPRSLRVLVENTLRAAAAAKAGSTGDSLRQAVLSANSLIAAATANGPVSEVEFRPTRLVLQDHSGIPVLADLASLRSIMADRGLDPRLARPALPVDLVVDHSVEVASWARPDALSLNMRREYELNGERYRFLRWAQQEISGLRVVPPGRGIVHQMHLEHLARVVLPDGDGLIAPDTVLGTDSHTPMVGALGVLGWGVGGIEAETVLLGHSVSLLSPQVIGVRLTGELPVGSTATDLALTMTEFLRNVGVVGRFVEFFGPGVCALSVPDRATLSNMAPEYGCTVAFFPVDDTTLRYLELTGRGAEEIRLVEAYCKEQGLFAGADASADPEFARVLDFDLSAVEPSAAGPRRPQDRLPLGTVPASFPEPSGAALAGAAEGRRELADGAVAIAAITSCTNTANPHSMVTAGLVARKAVQRGLAVPSWVKTSLAPGSRAVVHYLEAAGLLPDLEKLGFSVAGFGCTTCIGNSGGLAPEAEERARTEGVRLAAVLSGNRNFEGRIHPDVAAAYLASPALVVAYALAGTVLTDLTARPVGTDPDGAEVFLADLWPTPEEIDAAVGLVAREHFASEKESLFTGGPEWTSLEHPSGPVYDWPAESDYLLRSPFFDGLTHAPLSDLSGARVLVLTGDSTTTDHISPAGAIGADSPAGRYLRALGVEPALFNSYGCRRGNHEVLVRGTFANPKFRNLLTPDVQGSSTLHVPTGERMSVHEAALAYAAEQVPVVVVGGRDYGFGSSRDWAAKGPALLGVRAVLAKSFERIHRSNLIGMGIVPLEFLPDQDAGTLGLTGHEALDVIGLDGLVPRGTVTVRARSAEGEPVAEWRMLARVDTAGELEYVRRGGFLRSVASDLLDAEA

SEQ ID NO: 4 - protein, 352 aa, Streptomyces rubellomurinus
MTVSQYPDVAVRSDSPRPEASVLPAALELLESAGLTANWRIVAGQAPDGDGGRKPEAGLLPPFAEPGASAYGLARAARRAWGGTFSVIERRGPLVRSDGTVNVLLFRYELDMSDTGMEFESNSGEQLALVKTAAELGPREIPEQAALGIQAIAPDRVSEFVRTAVGMCRRLDVDDLVVAHKGNSIKHTDGLFALTAAETLESLAGAAGIAWRSLIIDHMAAELVRNPLDYRAILTHSSFADLLAAALAGQMTTDQLGVCTTYDPAGRPLFGHDPALDTSAPDADPERWRVRRLASTLAAGAALAEHLRLPPAGRALRRAAAELVRRDGGPIGWDQAVTTARKSLRAEGPARS

SEQ ID NO: 5 - protein, 365 aa, Streptomyces rubellomurinus
MRKHTVTLIAGDGSGPELVAAARTVLDTAAGSAGFHLDWDVRPAGQGALRSVGELLPASTLDSIRRNGVALKGPTTTPIGVGHRSVNITIRRELDLYACVRPVRVWPGAGAVDSGIDVVVVRENTEDVYSGLEYPADSDGAAVVSATLSEFGHADPGIAFSLKPITPGGSRRIVAFALDHARKNGRRKVEVGADVYRYPRTEGLFFEAARELGPEYPDIVTATVPVTVLCDSLVRDPADRDVLVLPNLYGDVISDVTAALVGGLGMAPGANIGDRTAVFEATHGSAPEFAGSNRLNPTALILSGCMLLDHLGEAPTAARIRAAVRSVLEEGRSVTFDLLPPDRAARAVTTTAYAEAVIERLRDIG

SEQ ID NO: 6 - protein, 433 aa, Streptomyces jeddahensis
MLKSMPWSSGPVACDPPADGPLKDGRLRSDSPRDRPETGPYERDVPVRTSQSTPFARIGRDPEESTAVTHPFLDLAPLTAAHFAAIEQRVARLLSTEQDVVITQGEALLPLEGCIRGAARPGTTALNVITGPYGQTFGNWLRDCGATVIDLAVPFHTAVTAAQIDEALTEHPEIDFVSLVHAEAATGNTNPVAEIGEVVRAHGALFMLDAVASVGAEPLLPDAWGVDLCVIGAQKAMGGPAGVSAVSVSERAWQRMAANPQAPRRSYLSLLDWKERWIDGGRKALLHAPAQLEMLALEACLERIEAEGLGALMTRHATAAAATRAGALALGGGLEPYVYEARDAAPVATTLRTPPGVDASEVVARALAADPVLPLVAGGGALSKEMIRVNHYGADATRGVVHSALAALGGALAESGLTVNLEAARRAVTDAWS

SEQ ID NO: 7 - protein, 426 aa, Escherichia coli
MNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 8 - protein, 367 aa, Salmonella enterica
MTSRNYLLLTPGPLTTSRTVKEAMLFDSCTWDDDYNIGVVEQIRQQLTALATASEGYTSVLLQGSGSYAVEAVLGSALGPQDKVLIVSNGAYGARMVEMAGLMGIAHHAYDCGEVARPDVQAIDAILNADPTISHIAMVHSETTTGMLNPIDEVGALAHRYGKTYIVDAMSSFGGIPMDIAALHIDYLISSANKCIQGVPGFAFVIAREQKLAACKGHSRSLSLDLYAQWRCMEDNHGKWRFTSPTHTVLAFAQALKELAKEGGVAARHQRYQQNQRSLVAGMRALGFNTLLDDELHSPIITAFYSPEDPQYRFSEFYRRLKEQGFVIYPGKVSQSDCFRIGNIGEVYAADITALLTAIRTAMYWTK

SEQ ID NO: 9 - protein, 246 aa, Homo sapiens
MTMDKSELVQKAKLAEQAERYDDMAAAMKAVTEQGHELSNEERNLLSVAYKNVVGARRSSWRVISSIEQKTERNEKKQQMGKEYREKIEAELQDICNDVLELLDKYLIPNATQPESKVFYLKMKGDYFRYLSEVASGDNKQTTVSNSQQAYQEAFEISKKEMQPTHPIRLGLALNFSVFYYEILNSPEKACSLAKTAFDEAIAELDTLNEESYKDSTLIMQLLRDNLTLWTSENQGDEGDAGEGEN

SEQ ID NO: 10 - protein, 247 aa, Homo sapiens
MVDREQLVQKARLAEQAERYDDMAAAMKNVTELNEPLSNEERNLLSVAYKNVVGARRSSWRVISSIEQKTSADGNEKKIEMVRAYREKIEKELEAVCQDVLSLLDNYLIKNCSETQYESKVFYLKMKGDYYRYLAEVATGEKRATVVESSEKAYSEAHEISKEHMQPTHPIRLGLALNYSVFYYEIQNAPEQACHLAKTAFDDAIAELDTLNEDSYKDSTLIMQLLRDNLTLWTSDQQDDDGGEGNN

SEQ ID NO: 11 - protein, 255 aa, Homo sapiens
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 12 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 13 - protein, 246 aa, Homo sapiens
MGDREQLLQRARLAEQAERYDDMASAMKAVTELNEPLSNEDRNLLSVAYKNVVGARRSSWRVISSIEQKTMADGNEKKLEKVKAYREKIEKELETVCNDVLSLLDKFLIKNCNDFQYESKVFYLKMKGDYYRYLAEVASGEKKNSVVEASEAAYKEAFEISKEQMQPTHPIRLGLALNFSVFYYEIQNAPEQACLLAKQAFDDAIAELDTLNEDSYKDSTLIMQLLRDNLTLWTSDQQDEEAGEGN

SEQ ID NO: 14 - protein, 245 aa, Homo sapiens
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 15 - protein, 248 aa, Homo sp. [sequence omitted]